SEQUENCE LISTING (5 sequences)

SEQ ID NO: 1 - DNA, 40 bp, synthetic construct
  misc_feature 1..40: note = aptamer
  source 1..40: mol_type = other DNA
tggatggtagctcggtcggggtgggtgggttggcaagtct

SEQ ID NO: 2 - DNA, 37 bp, synthetic construct
  misc_difference 1..37: note = nucleic acid motif
  misc_difference 3: note = a, c, t, g, unknown or other that may be 2'-methylazobenzene modified
  misc_difference 7: note = a, c, t, g, unknown or other that may be 2'-methylazobenzene modified
  misc_difference 11: note = a, c, t, g, unknown or other that may be 2'-methylazobenzene modified
  misc_difference 15: note = a, c, t, g, unknown or other that may be 2'-methylazobenzene modified
  source 1..37: mol_type = other DNA
gcngcgnctcngcgncgattattacgcgcgagcgcgc

SEQ ID NO: 3 - DNA, 40 bp, synthetic construct
  misc_feature 1..40: note = aptamer
  source 1..40: mol_type = other DNA
tggatgatagctcggtcggggtggatgggttggcaagtct

SEQ ID NO: 4 - DNA, 18 bp, synthetic construct
  misc_feature 1..18: note = aptamer
  source 1..18: mol_type = other DNA
ggatggtagctcggtcgg

SEQ ID NO: 5 - DNA, 14 bp, synthetic construct
  misc_feature 1..14: note = aptamer
  source 1..14: mol_type = other DNA
ggtgggtgggttgg

(19) United States
(12) Patent Application Publication
(10) Pub. No.: US 2024/0425867 A1
(43) Pub. Date: Dec. 26, 2024

(54) NUCLEIC ACID-BASED ASSEMBLY AND USES THEREOF

(71) Applicants: Rheinische Friedrich-Wilhelms-Universität Bonn, Bonn (DE); Max-Planck-Gesellschaft zur Förderung der Wissenschaften e.V., München (DE)

(72) Inventors: Michael Famulok, Bonn (DE); Deepak Prusty, Bonn (DE); Adam Volker, Bonn (DE); Stephan Irsen, Bonn (DE)

(21) Appl. No.: 18/648,227

(22) Filed: Apr. 26, 2024

Related U.S. Application Data
(63) Continuation of application No. 18/244,823, filed on Sep. 11, 2023, now abandoned.
Application No. 18/244,823 is a continuation of application No. 16/467,151, filed on Jun. 6, 2019, now abandoned.

(30) Foreign Application Priority Data
Dec. 7, 2016 (EP) 16202754.4

(51) Int. Cl. C12N 15/115; A61K 31/704; A61K 31/713; A61K 41/00; A61K 47/54; A61K 47/69

(52) U.S. Cl. CPC C12N 15/115; A61K 31/704; A61K 31/713; A61K 41/0042; A61K 47/543; A61K 47/549; A61K 47/6909; A61K 47/6949; C12N 2310/16; C12N 2310/3515; C12N 2310/531; C12N 2320/32

(57) ABSTRACT

The present invention relates to a nucleic acid-based assembly comprising: at least one nucleic acid aptamer, and at least one nucleic acid motif designed to physically capture a drug. The nucleic acid motif may comprise one or more photo-responsive moieties that effect the release of the drug upon irradiation. The aptamer and the nucleic acid motif each can be covalently linked to one or more lipids, and the lipid-modified aptamer and nucleic acid motif may form the assembly through noncovalent interaction. The invention further relates to use of the nucleic acid-based assembly in the treatment of cancer.

### CROSS-REFERENCE TO RELATED APPLICATIONS

This application is a continuation application of U.S. patent application Ser. No. 18/244,823 entitled “NUCLEIC ACID-BASED ASSEMBLY AND USES THEREOF.” filed on Sep. 11, 2023, which is a continuation application of U.S. patent application Ser. No. 16/467,151 entitled “NUCLEIC ACID-BASED ASSEMBLY AND USES THEREOF,” filed on Jun. 6, 2019, which is a National Stage Application under 35 U.S.C. § 371 and claims the benefit of International Patent Application No. PCT/EP2017/081933 entitled “NUCLEIC ACID-BASED ASSEMBLY AND USE THEREOF IN CANCER THERAPY,” filed on Dec. 7, 2017, which claims the benefit of priority to European Patent Application No. 16202754.4, filed on Dec. 7, 2016, each of which are herein incorporated by reference in their entirety for all purposes.

### SEQUENCE LISTING SUBMITTED VIA ELECTRONIC FILING SYSTEM

The instant application contains a Sequence Listing which has been filed electronically in XML format and is hereby incorporated by reference in its entirety. Said XML copy, created on Aug. 9, 2024, is named 110618-0835USC1-1408359_SL.xml and is 73,000 bytes in size. The submission of this replacement sequence listing removes the previously submitted xml and PDF.

### FIELD OF THE INVENTION

The present invention relates to aptamer-based drug-delivery systems and their use in therapeutic applications.

### BACKGROUND OF THE INVENTION

There is a compelling demand for improvements in the effectiveness in both the transport and specific release of therapeutic molecules. A powerful approach is the use of aptamer-based tumor targeting systems in combination with controlled release of active therapeutics through physiochemical responses to external stimuli such as pH, light, chemicals, or internal cell markers. Due to their advantages over other targeting reagents such as easy synthesis, low immunogenicity, and high target affinity, DNA aptamers have opened up new opportunities for cellular targeting and have been selected against various cancer types, including without limitation prostate, pancreatic, colon and breast cancer. However, aptameric molecular nanocarriers are often limited by inefficient cellular uptake and short intracellular half-life as they are naturally susceptible to nuclease-mediated degradation.

Progress has been made to improve serum half-life and cell internalization efficacy by functionalizing nanocarriers with aptamers that target specific surface proteins, for instance polymeric nanoparticles, liposomes, aptamer-drug conjugates, aptamer-antibody conjugates, and aptamer-functionalized quantum dots. However, the majority of these approaches entailed significant trade-offs between complicated assembly, suboptimal size, limited payload capacity, and some show insufficient serum stability and cell internalization efficacy. In the case of aptamer-drug conjugates, covalent linking of targeting units to cytotoxic agents is one possibility for efficient treatment, however attachment may alter their biological activity.

Several recent studies employed a native cell-targeting aptamer that was modified by additional nucleobases for drug intercalation as a dual factor for cell targeting and, simultaneously, as a cargo for drug transport. For example, U.S. Pat. No. 9,163,048 B2 describes a multifunctional nucleic-acid-based anticancer drug prepared by physically capturing an anticancer drug in a linear nucleic acid having a thiol group at the 5′-end, and chemically binding gold nanoparticles and a nucleic acid aptamer. The multi-functional nucleic acid-based anti-cancer drug uses A10 aptamer to achieve high targeting properties and high-concentration anti-cancer drugs and gold nanoparticles to enable dual therapy of thermal and chemical therapy. Yet, there is an inherent limitation to broader applicability for such architectures, especially when extended to other aptameric platforms for targeting different cell types, even a minor modification of the aptamer sequence with a drug loading unit might result in significant disruption of binding affinity. Moreover, demanding manufacturing processes are needed to provide such multifunctional nucleic-acid-based anticancer drugs. Additional issues include the triggered release of the active drug, the obstacles of tumor penetration and low structural stability.

The present invention provides a delivery system that facilitates manufacture and provides improved stability, cellular targeting and uptake.

### INCORPORATION BY REFERENCE

All publications, patents and patent applications mentioned in this specification are herein incorporated by reference to the same extent as if each individual publication, patent or patent application was specifically and individually indicated to be incorporated by reference.

### SUMMARY OF THE INVENTION

In an aspect, the invention provides a nucleic acid-based assembly comprising: (a) at least one nucleic acid aptamer; at least one nucleic acid motif designed to physically capture a drug, wherein the nucleic acid motif comprises one or more photo-responsive moieties that effect the release of the drug upon irradiation; and at least one lipid. In preferred embodiments, the at least one aptamer and the at least one nucleic acid motif each are covalently linked to at least one lipid, wherein the lipid-modified aptamer and lipid-modified nucleic acid motif form the assembly through noncovalent interaction. The at least one lipid can be any useful type of lipid. In some embodiments, the at least one lipid comprises a triglyceride, diglyceride, monoglyceride, fatty acid, steroid, wax, or any combination thereof. In some embodiments, each of the at least one lipid is selected from the group comprising C₈₋₂₄ saturated or unsaturated fatty acids. Each of the at least one lipid may comprise at least 2, 3, 4, 5, 6, 7, 8, 9, 10, 11, 12, 13, 14, 15, 16, 17, 18, 19, 20, 21, 22, 23, or 24 carbon atoms. In some embodiments, each of the at least one lipid is selected from the group consisting of C₈, C₁₀, C₁₂, C₁₄, C₁₆, C₁₈, C₂₀, C₂₂, and C₂₄ saturated and unsaturated fatty acid chains, and any combination thereof. For example, each of the at least one lipid may comprise a C₁₂-lipid chain.

In the nucleic acid-based assembly of the invention, the at least one aptamer and/or the at least one nucleic acid motif may each comprise a terminal lipid modification. The terminal lipid modification can include any useful number of lipids. In some embodiments, the terminal lipid modification comprises at least 1, 2, 3, 4, 5, 6, 7, 8, 9, or 10 lipids. In some embodiments, the terminal lipid modification comprises 1, 2, 3, 4, 5, 6, 7, 8, 9, or 10 lipids. In preferred embodiments, the terminal lipid modification comprises 3, 4, or 5 lipids. The terminal lipid modification can be attached at either terminus. In some embodiments, the terminal lipid modification is attached to the 5′-end.

In the nucleic acid-based assembly of the invention, the at least one aptamer may target any useful biomarker/antigen. In some embodiments, the at least one aptamer targets at least one of a tissue antigen, a cancer-antigen, a tumor-antigen, a cellular antigen, a membrane protein, a cellular receptor, a cell surface molecule, a lymphocyte-directing target, a growth factor, or any combination thereof. By way of non-limiting example, at least one aptamer may target at least one of 4-1BB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2,EphB3, FAP, Fibronectin, Folate Receptor, Ganglioside GM3, GD2, glucocorticoid-induced tumor necrosis factor receptor (GITR), gp100, gpA33, GPNMB, ICOS, IGFIR, Integrin av, Integrin αvβ, KIR, LAG-3, Lewis Y, Mesothelin, c-MET, MN Carbonic anhydrase IX, MUC1, MUC16, Nectin-4, NKGD2,NOTCH, OX40, OX40L, PD-1, PDL1, PSCA, PSMA, RANKL, ROR1, ROR2, SLC44A4, Syndecan-1,TACI, TAG-72, Tenascin, TIM3, TRAILR1, TRAILR2, VEGFR-1, VEGFR-2, VEGFR-3, and any combination thereof. Additional non-limiting biomarker targets envisioned by the invention are disclosed herein. The at least one aptamer may comprise more than one aptamer, may target more than one antigen. or both. For example, the at least one aptamer may comprise multiple aptamers to a single target. The at least one aptamer may comprise multiple aptamers specific for different target biomarkers. In some embodiments, the at least one aptamer targets the hepatocyte growth factor receptor (cMET). The sequence SEQ ID NO: 1 is an exemplary anti-cMet aptamer. The invention can employ SEQ ID NO: 1 or a functional variant thereof.

In the nucleic acid-based assembly of the invention, the at least one nucleic acid motif can include a motif that forms one or more hairpin loops. In some embodiments, the motif that forms the one or more hairpin loops comprises a 5′-GC rich oligodeoxynucleotide. In some embodiments, the one or more hairpin loops intercalate the drug.

The nucleic acid-based assembly of the invention can be configured to use any appropriate photo-responsive moiety. In some embodiments, the photo-responsive moiety comprises an azobenzene group. A non-limiting example of such azobenzene includes 2′-methylazobenzene. In some embodiments, the 2′-methylazobenzene comprises 2′,6′-dimethylazobenzene.

In the nucleic acid-based assembly of the invention, wherein the nucleic acid motif may comprise the nucleotide sequence 5′-GCNGCGNCTCNGCGNCGATTATTACGCGCGAGCGCGC-3′ (SEQ ID NO: 2) or a functional variant thereof. In some embodiment, N in the sequence is a 2′,6′-dimethylazobenzene-D-threoninol residue.

The nucleic acid-based assembly of the invention can be configured to deliver any appropriate drug. Non-limiting examples of drugs contemplated by the invention include a regulatory molecule, an antagomir, a small interfering RNA, a microRNA, a pharmaceutical drug, or any combination thereof. In some embodiments, the drug comprises an anti-cancer drug or cocktail thereof. In embodiments, the drug comprises a planar aromatic therapeutic agent such as doxorubicin.

The nucleic acid-based assembly of the invention can be stimulated to release the drug upon irradiation. For example, by visible light, ultraviolet light, or X-ray.

In the nucleic acid-based assembly of the invention, the at least one aptamer and the at least one nucleic acid motif are present in a useful ratio. In some embodiments, the ratio is in a range from ≥1:10 to ≤10:1, ≥1:5 to ≤5:1, or ≥1:2 to ≤3:2. In embodiments, the ratio is 1:1.

In a related aspect, the invention provides use of the nucleic acid-based assembly described herein as a medicament. The medicament can be used for the treatment of any appropriate disease. In preferred embodiments, the medicament is for use in the treatment of cancer, wherein optionally the cancer comprises a solid tumor. The cancer can be an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumors (GIST), glioblastoma, head and neck squamous carcinoma, leukemia, liver hepatocellular carcinoma, low grade glioma, lung bronchioloalveolar carcinoma (BAC), lung non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, uveal melanoma, or any combination thereof. Additional non-limiting types of cancer envisioned by the invention are disclosed herein.

In another related aspect, the invention provides use a nucleic acid-based assembly of the invention for the manufacture of a medicament. The medicament can be used for the treatment of any appropriate disease or disorder. In some embodiments, the medicament is for use in the treatment of cancer, wherein optionally the cancer comprises a solid tumor. The cancer can be an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumors (GIST), glioblastoma, head and neck squamous carcinoma (BAC), lung non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, uveal melanoma, or any combination thereof. Additional non-limiting types of cancer envisioned by the invention are disclosed herein.

In still another related aspect, the invention provides a pharmaceutical composition comprising as an active ingredient a nucleic acid-based assembly as described herein. The pharmaceutical composition can be used for the treatment of any appropriate disease or disorder. In some embodiments, the pharmaceutical composition is for use in the treatment of cancer. The cancer can be an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumors (GIST), glioblastoma, head and neck squamous carcinoma, leukemia, liver hepatocellular carcinoma, low grade glioma, lung bronchioloalveolar carcinoma (BAC), lung non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, uveal melanoma, or any combination thereof. Additional non-limiting types of cancer envisioned by the invention are disclosed herein.

In yet another related aspect, the invention provides a method of delivering a drug to a cell, comprising contacting the cell with a nucleic acid-based assembly as described herein and irradiating the cell. The cell may be a cultured cell, a diseased cell, a tumor cell, a cancer cell, or any combination thereof. Various non-limiting types of cancer envisioned by the invention are disclosed herein. In some embodiments, delivery of the drug to the cell kills the cell. Any useful drug, including cocktails and combinations, can be used for the method of the invention. Various non-limiting drugs envisioned by the invention are disclosed herein.

In an aspect the invention provides a method of treating a disease or disorder in a subject in need thereof, the method comprising the step of administering to the subject a therapeutically effective amount of a nucleic acid-based assembly or a pharmaceutical composition as provided herein. The nucleic acid-based assembly or pharmaceutical composition can be used for the treatment of any appropriate disease or disorder. In some embodiments, the nucleic acid-based assembly or pharmaceutical composition are used in the treatment of cancer. The cancer can be an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumors (GIST), glioblastoma, head and neck squamous carcinoma, leukemia, liver hepatocellular carcinoma, low grade glioma, lung bronchioloalveolar carcinoma (BAC), lung non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, uveal melanoma, or any combination thereof. Additional non-limiting types of cancer envisioned by the invention are disclosed herein.

### BRIEF DESCRIPTION OF THE DRAWINGS

The figures which follow serve to illustrate the invention in more detail but do not constitute a limitation thereof.

FIGS. 1A-B illustrate an assembly of the invention (FIG. 1A) and use of such assembly (FIG. 1B).

FIG. 2A illustrates 5-(1-Dodecynyl) modified 5′-DMT-2′-deoxyuridine-phosphoramidite 1. FIG. 2B illustrates ³¹P NMR spectra of lipid-modified 5′-DMT-2′-dU-phosphoramidite 1.

FIGS. 3A-B illustrate the predicted secondary structures of aptamers trCLN3. Two G-quadruplexes were predicted using GQRS Mapper. FIG. 3B: Schematic representation of the lipid-mediated self-assembly of cMet binding motif trCLN3-L4 (motif 3) and doxorubicin (DxR) binding motif DxR-L4 (motif 4) forms the micellar nanoconstrut assembly, which may be referred to as “HyApNc” herein. A non-cMet-binding mutant trCLN3.mut-L4 (motif mut-3) was used instead of motif-3, resulting in a mutated nanoconstruct HyApNc.mut. For DxR-L4 motif see FIG. 3A and Example 6.

FIGS. 4A-B illustrate the reverse-phase chromatograms of the lipid-functionalized aptamers and their sequences of (FIG. 4A) trCLN3-L4 and (FIG. 4B) trCLN3.mut-L4 crude synthetic product. Ultraviolet (UV) absorbance at 260 nm is monitored during elution. Fraction 1 (shown in A and B) eluted at ˜8 min is the non-lipidated version of the aptamer trCLN3 and trCLN3.mut whereas fraction 2 eluted approximately at ˜22 min corresponds to the lipid-functionalized aptamer.

FIGS. 5A-C illustrate ESI mass spectra of the HPLC-purified (FIG. 5A) native trCLN3 aptamer (FIG. 5B) its lipid-functionalized derivative trCLN3-L4 and (FIG. 5C) lipid-functionalized two point mutant trCLN3.mut-L4. The corresponding expected and observed molecular masses of the aptamers were: 12.567 and 12.568, respectively, in FIG. 5A; 14.385 and 14.385, respectively, in FIG. 5B; and 14.353 and 14.352, respectively, in FIG. 5C.

FIGS. 6A-C illustrate critical Micelle Concentrations (CMC) determination using 6Fam- and Atto647N-labeled motif 3 as FRET pairs in 1:1 ratio in a varied concentration range. FIG. 6A: Fluorescence emission spectra (λₑₓ=480 nm; λₑₘ=669 nm) for FRET assembled 6Fam-3/Atto647N-3 nanoconstructs. FIG. 6B: Magnification of the emission spectra in 1 μM-35 nM range. FIG. 6C: The change of intensity ratio I₆₆₉/I₅₂₀ at different motif-3 concentrations (error bars: n=2±SD).

FIGS. 7A-B illustrate CMC determination from the fluorescence of the pyrene probes incorporated to the hydrophobic lipid core of trCLN3-L4 aptameric nanoconstructs. FIG. 7A: Fluorescence emission spectra (λₑₓ=339 nm) of pyrene-loaded trCLN3-L4 nanoconstructs at a fixed pyrene concentration of 100 μM and different trCLN3-L4 concentrations. FIG. 7B: Variations of the intensity ratios I₄₇₅/I₃₇₃ as a function of trCLN3-L4 3 concentrations (error bars: n=2±SD).

FIGS. 8A-D illustrate assembly and characterization of the photo-switchable hybrid-aptameric nanoconstruct (HyApNc-DxR). FIG. 8A: Structures of the lipid-functionalized dU-phosphoramidite 1, the 2′,6′-dimethylazobenzene-D-threoninol residue 2, and doxorubicin DxR. Shapes used to represent 2 and DxR in FIG. 8B are shown next to the chemical structures. FIG. 8B: The lipid-functionalized anti-cMet aptamer trCLN3-L4 3 and its self-assembly into the corresponding trCLN3-L4 nanoconstruct (top); the lipid-functionalized DxR-carrier hairpin motif DxR-L4 motif 4 modified with 2′,6′-dimethylazobenzene 2, and the self-assembly of 3, 4, and DxR (depicted as oval shape) to form DxR-loaded HyApNc-DxR nanoconstruct (bottom). FIG. 8C: AFM images of the trCLN3-L4 (top) and HyApNc-DxR (bottom) nanoconstructs show the size and morphology of the corresponding nanoconstruct. Scale bar: 200 nm. FIG. 8D: Size distribution of the trCLN3-L4 (top) and HyApNc-DxR (bottom) nanoconstructs shows that the hybrid nanoconstructs HyApNc-DxR (bottom) are on average about 10 nm larger than the homogeneous trCLN3-L4 nanoconstructs (top).

FIGS. 9A-B illustrate TEM micrographs of the self-assembled trCLN3-L4 nanoconstructs with uranyl acetate staining. Scale bar: Black and white scale bars: FIG. 9A 50 nm and FIG. 9B 25 nm. Inset: 5× zoom image of the same region.

FIG. 10A illustrates a schematic of the filter retention assay in which varying concentrations of lipid-functionalized trCLN3 derivatives competed with constant amounts of radiolabeled trCLN3 in binding to the target cMet. FIG. 10B illustrates a binding curves of trCLN3 (●), trCLN3-L4 (▪), and trCLN3.mut-L4 (♦) to human cMet competing against γ-³²P-trCLN3 displaying the percentage of the maximum signal as a function of the amount of competing aptamer in a concentration range between 10⁻¹⁰ to 10⁻⁶ (error bars: n=2±SD).

FIGS. 11A-C illustrate PAGE analysis of the stability of trCLN3 aptamer and its lipid-functionalized derivatives in (FIG. 11A) 10% phosphate buffered saline (PBS) buffered fetal calf serum (FCS) and (FIG. 11B) 10% PBS buffered human blood serum (HBS). γ-³²P-ATP-labeled aptamer bands of the unmodified trCLN3 (row-I), trCLN3.mut (row-II), trCLN3-L4 (row-III) and trCLN3.mut-L4 (row-IV) respectively at different time intervals. Bands at the migration level of the 0 h sample represent 100% intact aptamer, whereas signals at lower positions depict decomposition products. FIG. 11C: Comparison of the degradation pattern of lipidated vs. non-lipidated motifs at different time point of 0.3 to 72 h. Aptamer band intensities were calculated from gels as in I)-IV), the percentage of intact aptamer was calculated and a curve was fitted to the resulting time course. The half-lives (t_{1/2}) of the selected aptamers were determined from the half-life curve fitting and are shown in brackets of the corresponding legends (error bars: n=2±SD).

FIGS. 12A-F illustrate switching behavior of the DxR binding motif. FIG. 12A: Schematic of lipid-modified hairpin-duplex motif with repetitive 5′-CG-3′ base pairs for DxR intercalation. The modified DxR-L4 motif 4 show the positions of 2′,6′-dimethylazobenzene (DMAB)-switches on a D-threoninol backbone marked with a cross (X)=2′,6′-dimethylazobenzene; and four lipid chains are attached to the 5′-end. FIG. 12B: Schematic of the switch mechanism mediated by DMAB photoswitch. FIG. 12C: UV/vis-spectrum of DxR-L4 motif 4 in a range between λ=300 and λ=420 nm, showing two sets of curves for the reversible photo switching of DMAB moiety for alternating irradiation with UV (solid lines) and visible light (vis., dotted lines). The absorption maximum lies at λ=345 nm. FIG. 12D: Analytical PAGE analysis of reversible switching 2′,6′-dimethylazobenzene functionalized DxR-L4 motif 4. FIG. 12E: Fluorescence emission spectra (λₑₓ=480 nm) of a DxR solution with increasing molar ratios of 4 in the range of 1-7 μM (0.1-0.7 equiv.) showing a reduction in fluorescence intensity of DxR with an increasing concentration of added motif 4. FIG. 12F: Comparison of fluorescence quenching of DxR with the DMAB-moiety in trans-(●) and in cis-(□) conformation (error bars: n=3±s.d.).

FIG. 13A illustrates DMT-protected phosphoramidite carrying a 2′,6′-dimethylazobenzene (2). FIG. 13B illustrates ESI mass spectra of the doxorubicin carrying DxR-L4 motif 4. The corresponding expected and observed molecular masses of the aptamers are shown at the side of the ESI mass spectrum.

FIG. 14 illustrates UV/Vis-absorbance of the corresponding supernatants and flow through washings after each centrifugation step (error bars: n=2±SD).

FIGS. 15A-B illustrate photocontrolled and thermal release of remaining DxR bound to motif 4 after removing unbound excess DxR from the solution by phenol/CHCl3 (ref. 6) monitored by high-performance liquid chromatography (HPLC) assay. FIG. 15A: HPLC chromatogram of the motif 4-DxR complex with and without UV exposure (dotted vs. solid line). The release curves of DxR were obtained by measuring the fluorescence at 590 nm using a fluorescence detector attached to the HPLC. After 5 minutes of UV irradiation, motif 4-DxR complex displayed a 63% reduction in fluorescence compared to nonirradiated samples. FIG. 15B: Release of DxR bound motif 4 incubated at 37° C. solely through self-diffusion at different times over 48 h (percentage of DxR bound to motif 4 at different incubation time are shown in brackets). 0 h sample represents 100% DxR bound to motif 4. A 20% reduction in fluorescence was observed for the motif 4-DxR complex which was incubated for 48 hours (●). The 48 h sample was then exposed to UV light for 5 minutes, which further reduced the fluorescence by 50% (▪) (error bars: n=2±SD).

FIGS. 16A-C illustrate FRET study of the formation of functional hybrid-nanoconstruct (HyApNc). FIG. 16A: Fluorescence emission spectra (λₑₓ=535 nm; λₑₘ=669 nm) for FRET assembled Atto647N-labeled trCLN3-L4 (3) and Atto550-labeled DxR-L4 motif (4) HyApNc formation. Atto647N-3 was kept constant at 5 μM with increasing equivalents of Atto550-4. FIG. 16B: Maximum fluorescence intensities at λ=669 nm (I₆₆₉) as a function of increasing concentration of 4 showing an increase in energy transfer (error bars: n=3±s.d.). Saturation is reached between 2.0 and 2.5 equivalents of Atto550-4. FIG. 16C: Comparison of the FRET signal (λₑₓ=535 nm; λₑₘ=669 nm) of HyApNc consisting of 4 (straight) and 4 without the lipid tail (a550-4_{w/oL4}; dashed).

FIG. 17 illustrates FRET efficiency comparision for (λex=554 nm; λem=669 nm) HyApNc consisting of motifs Atto550-4 and Atto647-3 without (˜27%, F5) and with lipid tail (92%, F6). Mutated nanoconstructs (HyApNc.mut) consisting of Atto647.mut-3 motif and Atto550-4 exhibited similar FRET effect as shown by HyApNc (˜97%, F7) (error bars: n=3±SD).

FIGS. 18A-C illustrate time-resolved spectra of FRET micellar nanoconstructs in (FIG. 18A) 95% human blood serum (HBS) and (FIG. 18B) 1 mM bovine serum albumin solution (BSA). FIG. 18C: Time traces of the FRET ratio=I669/(I669+I576), in human blood serum (●) and in solutions of bovine serum albumin (BSA) (●) (n=2, mean±SD plotted).

FIGS. 19A-B illustrate fluorescence microscopy (top) and flow cytometry analysis (bottom) of binding or internalization of atto 647-modified aptamer trCLN3 FIG. 19A: Confocal images of NCI-H1838 cells incubated with I) Atto647N-3 at 37° C. II) Atto647N-3 at 4° C. Arrow: Alexa488-WGA membrane stain (lower cell outlines) shows colocalization with Atto647N-3 (upper). III) Atto647N.mut-3 at 37° C. IV) Atto647N-trCLN3_{w/oL4} (without lipid-modification) at 37° C. Merged (bottom) and unmerged (top) confocal images of H1838 cells incubated with Atto647N labeled trCLN3-L4 nanoconstructs (A647N-3; upper; c3). Cells were membrane stained with Alexa488 WGA (lower cell outlines; c2), nuclei were stained with Hoechst 33342 (lower filled circular entities; c1) and analyzed for Atto647N-3 uptake (shown in upper panels; c3). Scale bars: 50 μm. FIG. 19B: FACS histograms for cells treated with Atto647N-3 at 37° C. (“a647-c. 37° C.”) showed a significant shift in Atto647 fluorescence intensity compared to cells treated with Atto647N-3 at 4° C. (“a647-c. 4° C.”) thus confirming the endocytotic internalization pathway. A minimal shift in Atto647 fluorescence intensity was observed for cells treated with either a scrambled aptamer Atto647N.mut-3 (“a647-mut 3”) or with Atto647N-trCLN3_{w/oL4} (dashed line) at 37° C. compared to untreated cells (“Control”), confirming a marginal internalization due to non-specific binding or lack of lipidation.

FIG. 20 shows merged (bottom) and unmerged (top) confocal images of NCI-H1838 cells incubated with Atto647N labeled trCLN3-L4 nanoconstructs (A647N-3; upper; c3) having end concentrations a) 10 μM b) 1 μM and c) 0.2 μM at 37° C. Cells were membrane stained with Alexa488 WGA (lower, cell outlines; c2), nuclei were stained with Hoechst 33342 (lower, filled circular entities; c1) and analyzed for Atto647N-3 uptake (shown alone in upper panels; c3). The arrow shows a punctuated fluorescent pattern in figure b, which indicates that the A647N-3 nanoconstructs might localize in the endosomes.

FIGS. 21A-F illustrates confocal fluorescence images of H1838 cells treated with the HyApNc consisting of Atto550-DxR-L4 motif (A550-4) and Atto647N-trCLN3-L4 (A647N-3) motifs in 1:1 ratio. Both A647N-3 (FIG. 21A; c2) and A550-4 (FIG. 21B; c3) fluorescence were observed from the cytosol including a FRET-mediated Atto647N signal (FIG. 21C; c4). Calculated FRET signal from reconstructed FRET images (FIG. 21D) indicate the intracellular integrity of the functional nanoconstruct (HyApNc). FIGS. 21E-F overlay images of cells incubated with HyApNc (FIG. 21E; A647N-3+A550-4), and HyApNc.mut (FIG. 21F; A647N.mut-3+A550-4) as a negative control with Atto647N-labeled mutant trCLN3.mut-L4 motif (scale bar: 50 μm) (FIG. 21F; c4). The complete overlay sets for e and f are shown in FIG. 17. Aptamer constructs were incubated at 37° C. for 2 h, followed by membrane staining with Alexa488-WGA (cell outlines), and nuclei staining with Hoechst 33342 (filled circular entities).

FIG. 22 shows confocal microscopy images of H1838 cells after incubation with (a; upper panels) HyApNc (a647N-3+a550-4) and (b; lower panels) HyApNc.mut (A647N.mut-3+A550-4) as a negative control. Both Atto647N (a; c2) and Atto550 (b; c3) fluorescence were observed from the cytosol including a FRET-mediated Atto647N signal, where the cells were incubated with HyApNc. In contrast. the mutilated functional nanoconstruct with Atto647N-labeled mutant trCLN3-L4 (A647N-mut 3, lower panels) resulted in a very weak fluorescence signal for both dyes inside cells (FIGS. 22, c2 and c3) including a poor FRET signal. Reconstructed calculated FRET images for HyApNc (row 1, column 5) and HyApNc.mut (row 2, column 5) are given respectively.

FIG. 23A: Time dependent growth inhibition assay (MTT) for H1838 cells exposed to UV light at 365 nm for 0 (●), 5 (▪), 10 (▴), 15 (▾) and 30 (♦) minutes at a fixed intensity of 350 mW/cm². FIG. 23B: Relative cell viability of H1838 cells at different cell densities under different irradiation times (error bars: n=2±SD). Bars from left to right for each density: 0, 5, 10, 15 and 30 minutes irradiation.

FIGS. 24A-C illustrates confocal microscopy (top) and FACS analysis (bottom) of the H1838cells. 2 h after incubation with the DxR-loaded HyApNc nanoconstructs without or with UV triggering. FIG. 24A: Confocal image of intracellular distribution of DxR released from HyApNc (central row, c2) in the H1838 cells incubated with I) free DxR, II) HyApNc-DxR not exposed to UV-irradiation, III) HyApNc-DxR exposed to UV-light (λ=365 nm, 350 mW/cm²). IV) HyApNc_{w/o Az}-DxR without UV-irradiation and V) HyApNc_{w/o Az}-DxR exposed to UV-light (λ=365 nm, 350 mW/cm²) (Scale bar: 50 μm). Signal from C1 (upper row) and C2 (central row) show the fluorescence of Hoechst 33342 and DxR (nuclei staining) respectively. The overlay (C1+C2, lower row) shows colocalization of Hoechst 33342 and DxR. An increase in nuclear accumulation of DxR upon light triggering was observed only for the photoactivated nanoconstruct. FIGS. 24B-C: Flow cytometry histogram showing quantitative comparison of DxR accumulation in H1838 cells after incubation with indicated constructsat 37° C. for 2 h. FIG. 24B: free DxR (“Free DxR”), mutant non-targeted nanoconstructs HyApNc.mut-DxR (“HyApNc. (mut)-DxR”), targeted nanoconstructs HyApNc-DxR without UV (central solid line), or with UV irradiation (central dotted line) FIG. 24C: HyApNc_{w/oAz}-DxR without UV (central solid line) or with UV irradiation (central dotted line). The concentration of DxR either in free form or its equivalent in complex form in the cell culture kept fixed at 8 μM. Untreated cells were shown in peak labeled “Control”. The numbers in bracket of the legends are the geometric mean of the corresponding peaks.

FIGS. 25A-C illustrates cell viability (MTT) assays of DxR-loaded nanoconstructs in cMet positive NCI-H1838 cells. FIG. 25A: Cytotoxicities of HyApNc-DxR and HyApNc.mut-DxR complexes in combination with the UV irradiation at the indicated DxR concentrations (0.125-50 μM ranges). As a control, viabilities of the cells treated with free DxR alone and HyApNc-DxR complex without UV irradiation were compared (error bars: n=2±SD). FIG. 25B: 8 h post incubation MTT assays where an increasing number of H1838 cells treated with (i) unloaded HyApNc (●, ▪) (ii) photoactive HyApNC-DxR (▴, unfilled ▾) and (iii) photo-inactive HyApNC_{w/o Az}-DxR (⋄, ▾) with and without subsequent UV irradiation (dotted vs. solid line, respectively). As control, cell viabilities of the H1838 cells treated with Roswell Park Memorial Institute (RPMI) medium with 10% FCS and not exposed to UV irradiation (●) were measured at 570 nm (error bars: n=2±SD). FIG. 25C: Time dependent cytotoxicities of photoactive HyApNC-DxR (▴, unfilled ▾) against photo-inactive HyApNC_{w/o Az}-DxR (⋄, ▾) with and without UV irradiation (dotted vs. solid lines, respectively), where the cells were treated with the DxR-complex for various incubation time of 8 h, 24 h, and 48 h respectively before being subjected to the MTT assay (error bars: n=2±SD).

### DETAILED DESCRIPTION OF THE INVENTION

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present specification will control.

An alternative and highly versatile approach to minimize drawbacks with current aptamer drug delivery systems is to incorporate a cell-targeting aptamer unit and separate drug-carrying functionalities into a single multi-functional nano-assembly. As desired, these units can be anchored onto a single nanoscaffold through non-covalent interactions, enabling convenient self-assembly of tunable modular components. In some instances, simple mixing of the two, or more, moieties can spontaneously self-assemble to form a single nanoconstruct containing these motifs. Accordingly, the invention solves problems with current aptamer-based drug delivery systems by providing a nucleic acid-based assembly. The assembly comprises at least one nucleic acid aptamer, and at least one binding agent designed to physically capture a drug and release it upon a signal. As a non-limiting example, the binding agent can be a nucleic acid motif. The nucleic acid motif may comprise one or more photo-responsive moieties that effect the release of the drug upon irradiation. To form the assembly, the aptamer and the nucleic acid motif may be covalently linked to one or more lipids. In some embodiments, the lipid-modified aptamer and nucleic acid motif form the assembly through noncovalent interaction.

It was found that the lipid-functionalized aptamer and nucleic acid motif provide a highly versatile nano-level assembly, which forms by spontaneous self-assembly by simple mixing of the lipid-modified aptamer and nucleic acid motif. See Examples herein. The invention advantageously provides a multi-functional assembly that can encompass a cell-targeting aptamer unit and a separate nucleic acid motif with drug loading sites, where both are held together within a single nano-size scaffold through noncovalent interactions. The design of the assembly allows using a large variety of lipid-modified aptamers or molecules that can self-assemble into a functional nano-size assembly. This provides for a highly versatile applicability. The assembly further provides good nuclease stability, and high target binding affinity and cellular uptake. These features advantageously allow a wide applicability for the simultaneous delivery of a variety of different regulatory molecules, such as antagomirs, small interfering RNAs, microRNAs, and pharmaceutical drugs with high specificity and efficiency.

The lipid-modified aptamer and nucleic acid motif can self-assemble to form hybrid heterogeneous nanoconstructs of roughly spherical geometry when the lipid modifications are present. The lipid-modified aptamer and nucleic acid motif can form an assembly of spherical or essentially spherical geometry, particularly a hybrid micellar construct. The size of the assembly may result from the physico-chemical properties of the aptamer and the nucleic acid motif, or from structural differences, or both. The size of the assembly further may depend on the lipid. Using biocompatible lipids the size of the assembly advantageously may be that of a nano-level structure. In some embodiments, the assembly has an average diameter in a range from ≥5 nm to ≤100 nm, for example, in a range from ≥10 nm to ≤70 nm, in a range from >15 nm to ≤50 nm, or in a range from >20 nm to ≤40 nm. For example, the assembly may have an average diameter from ≥10 nm, ≥15 nm, ≥20 nm ≥25 nm, ≥30 nm, ≥40 nm, ≥50 nm, ≥60 nm, >70 nm, ≥ 80 nm, or >90 nm, and an average diameter ≤15 nm, ≤20 nm, ≤25 nm, ≤30 nm, ≤40 nm, ≤50 nm, ≤60 nm, ≤70 nm, ≤80 nm, ≤90 nm, or ≤100 nm. In some embodiments, the assembly has an average diameter in a range from >20 nm to ≤40 nm. The term “average diameter” refers to the average value of all diameters or arithmetically averaged diameters, relative to all particles.

In some embodiments, the assembly is capable of self-assembly. A self-assembled aggregation advantageously can be effected by simple mixing of the lipid-modified aptamer and nucleic acid motif. The lipid-modification not only provides for self-assembled aggregation of micellar nanostructures, but chemically linking the aptamer and the nucleic acid motif to biocompatible lipids also can improve uptake efficiency and reduce nuclease-mediated degradation of the assembly in a cell. The assembly, which is held together through noncovalent interaction, further showed good integrity. It could be shown that the self-aggregated nanoconstructs were stabilized in aqueous solution through hydrophobic interaction of the lipids. See, e.g., Examples 4-5, 7 herein. Such self-assembled structures even offer an unprecedented degree of control over the ratio of different functional domains based on the therapeutic requirements.

As used herein, the term “at least one” nucleic acid aptamer or nucleic acid motif particularly refers to the species of the aptamer and nucleic acid motif, and is not intended to limit the number of aptamer molecules and nucleic acid motif molecules comprised in the assembly. The assembly may comprise a multitude of each of aptamer and nucleic acid motif. For example, the assembly may comprise at least 1, 2, 3, 4, 5, 6, 7, 8, 9, 10, 15, 20, 25, 30, 40, 50, 60, 70, 80, 90, 100, 200, 300, 400, 500, 600, 700, 800, 900 or at least 1000 aptamer molecules. For example, the assembly may further comprise at least 1, 2, 3, 4, 5, 6, 7, 8, 9, 10, 15, 20, 25, 30, 40, 50, 60, 70, 80, 90, 100, 200, 300, 400, 500, 600, 700, 800, 900 or at least 1000 nucleic acid motifs. The ratio of aptamer and nucleic acid motif can be tuned to meet desired characteristics, e.g., by adjusting the concentration of molecules introduced during assembly.

The present invention will be further described in connection with various embodiments and other aspects. They may be combined freely unless the context clearly indicates otherwise.

The lipid may be an aliphatic hydrocarbon or fatty acid, including as non-limiting examples, C₈-C₂₄-alkanes, C₈-C₂₄-alkenes, and C₈-C₂₄-alkynes, and particularly may be selected from saturated and unsaturated fatty acids. The lipids used in the assembly may comprise triglycerides (e.g. tristearin), diglycerides (e.g. glycerol bahenate), monoglycerides (e.g. glycerol monostearate), fatty acids (e.g. stearic acid), steroids (e.g. cholesterol), and waxes (e.g. cetyl palmitate). Preferably, the lipid-modification may be the covalent binding to a C₈₋₂₄ saturated or unsaturated fatty acid chain. The saturated or unsaturated fatty acid chain may comprise any appropriate number of carbon atoms. In various embodiments, the saturated or unsaturated fatty acid chain comprises at least 2, 3, 4, 5, 6, 7, 8, 9, 10, 11, 12, 13, 14, 15, 16, 17, 18, 19, 20, 21, 22, 23, or at least 24 carbon atoms. In some embodiments, the saturated or unsaturated fatty acid chain comprises between 8 and 24 carbon atoms, e.g., 10 to 18 carbon atoms, or 12 to 16 carbon atoms. In embodiments, the lipid is selected from the group consisting of C₈, C₁₀, C₁₂, C₁₄, C₁₆, C₁₈, C₂₀, C₂₂, and C₂₄ saturated and unsaturated fatty acid chains. Biocompatible lipids advantageously can improve uptake efficiency of the assembly. Further, fatty acid chains provide an effectively linear lipophilic chain, which supports the formation of regular micelles. In preferred embodiments, the lipid-modification is provided by C₁₂-lipid chains. It was observed that the C₁₂ lipid modification attached to the 5′-end of the aptamer induced self-aggregation of spherical micellar nanoconstructs at a concentration above the critical micelle concentration in aqueous solution. See, e.g., Examples 3-4 herein.

The lipids may be covalently linked directly with the nucleic acids of the aptamer or the nucleic acid motif. Lipid-modified oligo (deoxy) nucleotides are commercially available. Or lipid modifications can be synthesized chemically. Nucleotides synthesized with a thio group can be coupled to maleimide-functionalized lipids, while nucleotides bearing a carboxylic acid or amine functionality can be coupled to an amine-or carboxylic acid-functionalized lipid. In embodiments, lipid-modified aptamers and nucleic acid motifs may be synthesized using lipid-modified phosphoramidites with a C₁₂-lipid chain incorporated at the 5-position of, for example, uridine-phosphoramidite. These modified bases may be attached to the nucleic acids, thereby introducing lipid tails into the aptamer and/or the nucleic acid motifs. Preferred is a terminal lipid modification of the aptamer and/or nucleic acid motif at the 3′ and/or 5′-end. A terminal modification has the advantage of supporting the formation of spherical micellar structures. Further, the synthesis of a lipid-modified nucleic acid sequence that is modified only terminally can be carried out with commercially available monomers, and synthesis protocols known in the prior art can be used. A lipid modification preferably is provided at the 5′-end of the aptamer or the nucleic acid motif. The coupling of lipid-modified amidites to the 5′-end of nucleic acids can be incorporated when the nucleic acid is synthesized, for example by the process of amidite chemistry. In some embodiments, the lipid modification is provided at the 5′-end of the nucleic acid by specially modified phosphoramidites following a phosphoramidite process for the synthesis of the nucleic acid. For example, 5-(1-dodecynyl)-modified-2′-deoxyuridine-phosphoramidite groups may be used.

The aptamer and the nucleic acid motif each can be covalently linked to one or more lipids. In embodiments, the lipid-modified aptamer and/or nucleic acid motif are covalently linked to any appropriate number of lipids. In preferred embodiments, the lipid-modified aptamer and/or nucleic acid motif are covalently linked to any number between 1 to 10 lipids, preferably 2 to 8, 2 to 6, or 3 to 5, lipids. As desired, the lipid-modified aptamer and/or nucleic acid motif can be covalently linked to 1, 2, 3, 4, 5, 6, 7, 8, 9, 10, 12, 15 or 20 lipids. The lipid-modified aptamer and/or nucleic acid motif can be covalently linked to at least 1, 2, 3, 4, 5, 6, 7, 8, 9, 10, 12, 15 or 20 lipids. In some embodiments, the lipid-modified aptamer and/or nucleic acid motif are covalently linked to 2 to 6, preferably to 3, 4 or 5 lipids. The lipids may be covalently linked directly with the respective nucleic acid. In some embodiments, four lipids, such as C₁₂-lipid chains, are attached to the 5′-end of the aptamer and/or the nucleic acid motif. In embodiments, four C₁₂-lipid modified deoxyuridine residues are attached to the 5′-end. It could be shown that the aptamer and the nucleic acid motif self-assemble to form hybrid heterogeneous nanoconstructs of approximately spherical geometry when the lipid modifications are present. See, e.g., Example 4 herein. The aptamer and/or the nucleic acid motif may comprise a terminal lipid modification with any appropriate number of terminal lipids. As a non-limiting example, the aptamer and/or the nucleic acid motif comprise a terminal lipid modification preferably in a range from 1 to 10 lipids, preferably 2 to 8, 2 to 6, or 3 to 5 lipids attached to the 5′-end. The terminal lipid modification may comprise 1, 2, 3, 4, 5, 6, 7, 8, 9, 10, 12, 15, 20 or other appropriate number of lipids. The ability to form nanoconstructs due to lipidation, and the lipidation providing for efficient uptake into cancer cells are advantages of the assembly. Without being bound by theory, such lipidation may provide for cellular uptake via an endocytotic uptake mechanism.

As described herein, the nucleic acid-based assembly comprises at least one nucleic acid aptamer. As used herein, the term “nucleic acid aptamer” refers to an oligonucleotide molecule that binds to a specific target molecule. Conventially aptamers refer to molecules that bind to their targets through other than Watson-Crick base pairing. Aptamers can be identified that bind to the target of interest with high affinity, for example in the low nano molar range. The aptamer can be provided in the form of a single-stranded DNA or RNA molecule, or chemically modified versions thereof. Various chemical modifications can be introduced that effect desired properties. In some embodiments, the aptamer comprises a deoxyribonucleotide and/or a 2′-F 2′-deoxy modified sequence. Such modification may enhance stability. The nucleic acid aptamer provides a cell-targeting property to the assembly. Such targeting can be chosen to minimize effects of the drug on non-target cells.

The invention encompasses use of aptamers targeting various proteins preferably expressed on the surface of a target cell, including without limitation cancer biomarker proteins. In some embodiments, aptamers are chosen that specifically bind to cancer cells expressing or over-expressing proteins specific for a certain tumor on the cellular surface. In some embodiments, aptamers are chosen that bind to single cancer cell types, e.g., an aptamer to a prostate biomarker may target prostate cancer cells, an aptamer to a breast cancer marker may target breast cancer cell, etc. Alternately, aptamers may be chosen that target cancer cells regardless of anatomical origin. Various known cancer-specific aptamers can be used for the assembly of the invention. In addition, aptamers to desired cellular targets can be evolved by the systematic evolution of ligands by exponential enrichment (SELEX) process. See, e.g., U.S. Pat. Nos. 5,270,163, 5,475,096, 5,567,588, 5,670,637, 5,683,867, 5,705,337, 5,763,177, 5,789,157, 5,789,163, 5,843,653, 5,853,984, 6,506,887, 6,706,482, 7,947,447, and 8,071,288; each of which patents is incorporated by reference herein in its entirety. In some embodiments, the cell-SELEX approach using whole live cells as targets to select aptamers for cell recognition. See, e.g., U.S. Pat. Nos. 5,763,566, 5,864,026, 5,789,157, 5,712,375, and 6,114,120; each of which patents is incorporated by reference herein in its entirety. For additional discussion of SELEX and its applications, see, e.g., Klug and Famulok. All you wanted to know about SELEX. Mol Biol Rep. 1994, Vol. 20 (2), p. 97-107; Dua P. et al. Patents on SELEX and therapeutic aptamers. Recent Pat DNA Gene Seq. 2008; 2 (3):172-86; Huang et al. Integrated microfluidic system for rapid screening of CRP aptamers utilizing systematic evolution of ligands by exponential enrichment (SELEX). Biosens Bioelectron. 2010, Vol. 25 (7), p. 1761-6; Mayer et al. Fluorescence-activated cell sorting for aptamer SELEX with cell mixtures. Nat Protoc. 2010, Vol. 5 (12), p. 1993-2004; Sefah et al., Development of DNA aptamers using Cell-SELEX. Nat Protoc. 2010 June; 5 (6):1169-85; Zhang Y et al., Aptamers selected by cell-SELEX for application in cancer studies. Bioanalysis. 2010 May; 2 (5):907-18; Arnold, S, et al. One round of SELEX for the generation of DNA aptamers directed against KLK6. Biol Chem. 2012 Apr. 1; 393 (5):343-53; Graham JC and Zarbl H (2012) Use of Cell-SELEX to Generate DNA Aptamers as Molecular Probes of HPV-Associated Cervical Cancer Cells. PLOS ONE 7 (4); Ohuchi, Cell-SELEX Technology; BioResearch, 1 (6):265-272 (2012); Ruff, et al, Real-Time PCR-Coupled CE-SELEX for DNA Aptamer Selection. ISRN Molecular Biology, vol. 2012; Ye et al., Generating aptamers by cell-SELEX for applications in molecular medicine. Int J Mol Sci. 2012; 13 (3):3341-53; each of which references is incorporated by reference herein in its entirety.

The SELEX method encompasses the identification of high-affinity nucleic acid ligands containing modified nucleotides conferring improved characteristics on the ligand, such as improved in vivo stability or improved delivery characteristics. Alternately, identified aptamers can be modified to provide desired properties. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5′ position of pyrimidines, and 8′ position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH₂), 2′-fluoro (2′-F), and/or 2′-O-methyl (2′-OMe) substituents.

Modifications of the nucleic acid aptamers contemplated for use in the assembly of the invention include, but are not limited to, those which provide other chemical groups that incorporate additional charge, polarizability, hydrophobicity, hydrogen bonding, electrostatic interaction, and fluxionality to the nucleic bases or to the nucleic acid aptamer as a whole. Modifications to generate oligonucleotide populations which are resistant to nucleases can also include one or more substitute internucleotide linkages, altered sugars, altered bases, or combinations thereof. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping.

In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotides through an —O—, —N—, or —S— linkage. Not all linkages in the oligonucleotide are required to be identical. As used herein, the term phosphorothioate encompasses one or more non-bridging oxygen atoms in a phosphodiester bond replaced by one or more sulfur atoms.

The nucleic acid aptamers may comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). Other modifications are known to one of ordinary skill in the art. Such modifications may be pre-SELEX process modifications or post-SELEX process modifications (modification of previously identified unmodified ligands) or may be made by incorporation into the SELEX process.

Pre-SELEX process modifications or those made by incorporation into the SELEX process yield nucleic acid aptamers with both specificity for their target and improved stability, e.g., in vivo stability. Post-SELEX process modifications made to nucleic acid aptamers may result in improved stability without adversely affecting the binding capacity.

The SELEX method encompasses combining selected oligonucleotides with other selected oligonucleotides and non-oligonucleotide functional units as described in U.S. Pat. No. 5,637,459 and U.S. Pat. No. 5,683,867. The SELEX method further encompasses combining selected nucleic acid ligands with lipophilic or non-immunogenic high molecular weight compounds, as described, e.g., in U.S. Pat. Nos. 6,011,020, 6,051,698, and PCT Publication No. WO 98/18480. These patents and applications describe the combination of a broad array of shapes and other properties, with the efficient amplification and replication properties of oligonucleotides, and with the desirable properties of other molecules.

The aptamers with specificity and binding affinity to the target(s) of the present invention can be selected by the SELEX N process as described herein. As part of the SELEX process, the sequences selected to bind to the target are then optionally minimized to determine the minimal sequence having the desired binding affinity. The selected sequences and/or the minimized sequences are optionally optimized by performing random or directed mutagenesis of the sequence to increase binding affinity or alternatively to determine which positions in the sequence are essential for binding activity. Additionally, selections can be performed with sequences incorporating modified nucleotides to stabilize the aptamer molecules against degradation in vivo.

Aptamer resistance to nuclease degradation can be greatly increased by the incorporation of modifying groups at the 2′-position. Fluoro and amino groups have been successfully incorporated into oligonucleotide pools from which aptamers have been subsequently selected. However, these modifications greatly increase the cost of synthesis of the resultant aptamer, and may introduce safety concerns in some cases because of the possibility that the modified nucleotides could be recycled into host DNA by degradation of the modified oligonucleotides and subsequent use of the nucleotides as substrates for DNA synthesis. Aptamers that contain 2′-O-methyl (“2′-OMe”) nucleotides may overcome one or more potential drawbacks. Oligonucleotides containing 2′-OMe nucleotides are nuclease-resistant and inexpensive to synthesize. Although 2′-OMe nucleotides are ubiquitous in biological systems, natural polymerases do not accept 2′-OMe NTPs as substrates under physiological conditions, thus there are no safety concerns over the recycling of 2′-OMe nucleotides into host DNA. The SELEX method used to generate 2′-modified aptamers is described, e.g., in U.S. Provisional Patent Application Ser. No. 60/430,761, filed Dec. 3, 2002, U.S. Provisional Patent Application Ser. No. 60/487,474, filed Jul. 15, 2003, U.S. Provisional Patent Application Ser. No. 60/517,039, filed Nov. 4, 2003, U.S. patent application Ser. No. 10/729,581, filed Dec. 3, 2003, and U.S. patent application Ser. No. 10/873,856, filed Jun. 21, 2004, entitled “Method for in vitro Selection of 2′-O-methyl substituted Nucleic Acids”, each of which is herein incorporated by reference in its entirety.

The construct of the invention can be directed to the desired cells or tissue using one or more aptamer directed to a useful target biomarker. For example, the choice of target biomarker can be made depending on a type of cell, such as a cancer antigen/biomarker to target cancer cells or a tissue antigen/biomarker to target cells from a particular tissue. Such cancer biomarkers might be a marker of a specific origin or form of cancer, or might be a marker of neoplastic cells of multiple origins. Multiple aptamers may be used to direct the constructs to cellular targets as desired. Accordingly, a single construct can be targeted to different cells having different antigens or biomarkers. Muliple aptamers may also serve to enhance targeting of a single cell by targeting multiple antigens or biomarkers of such cell.

In some embodiments, the target biomarker of the one or more aptamer is selected from the group consisting of CD19, CD20, CD21, CD22 (also known as LL2), CDIM, Lym-1, and any combination thereof. In some embodiments, the target biomarker of the one or more aptamer comprises a membrane associated protein. In embodiments, the membrane associated protein is selected from the group consisting of CD4, CD19, DC-SIGN/CD209, HIV envelope glycoprotein gp120, CCR5, EGFR/ErbB1, EGFR2/ErbB2/HER2, EGFR3/ErbB3, EGFR4/ErbB4, EGFRVIII, Transferrin Receptor, PSMA, VEGF, VEGF-2, CD25, CD11a, CD33, CD20, CD3, CD52, CEA, TAG-72, LDL receptor, insulin receptor, megalin receptor, LRP, mannose receptor, P63/CKAP4 receptor, arrestin, ASGP, CCK-B, HGFR, RON receptor, FGFR, ILR, AFP, CA125/MUC16, PDGFR, stem cell factor receptor, colony stimulating factor-1 receptor, integrins, TLR, BCR, BAFF-R, and any combination thereof. The target biomarker of the one or more aptamer can be a cellular receptor selected from the group consisting of nucleolin, human epidermal growth factor receptor 2 (HER2), CD20, a transferrin receptor, an asialoglycoprotein receptor, a thyroid-stimulating hormone (TSH) receptor, a fibroblast growth factor (FGF) receptor, CD3, the interleukin 2 (IL-2) receptor, a growth hormone receptor, an insulin receptor, an acetylcholine receptor, an adrenergic receptor, a vascular endothelial growth factor (VEGF) receptor, a protein channel, cadherin, a desmosome, a viral receptor, and any combination thereof. In various embodiments, the target biomarker of the one or more aptamer is a cell surface molecule selected from the group consisting of IgM, IgD, IgG, IgA, IgE, CD19, CD20, CD21, CD22, CD24, CD40, CD72, CD79a, CD79b, CD1d, CD5, CD9, CD10, CD1d, CD23, CD27, CD38, CD48, CD80, CD86, CD138, CD148, and any combination thereof. The target biomarker can be a lymphocyte-directing target such as a T-cell receptor motif, T-cell a chain. T-cell β chain. T-cell y chain. T-cell A chain, CCR7, CD3, CD4, CD5, CD7, CD8, CD11b, CD11c, CD16, CD19, CD20, CD21, CD22, CD25, CD28, CD34, CD35, CD40, CD45RA, CD45RO, CD52, CD56, CD62L, CD68, CD80, CD95, CD117, CD127, CD133, CD137 (4-1 BB), CD163, F4/80, IL-4Ra, Sca-1, CTLA-4, GITR, GARP, LAP, granzyme B, LFA-1, transferrin receptor, and any combination thereof.

In some embodiments, the target biomarker of the one or more aptamer comprises a growth factor. The growth factor can be selected from the group consisting of vascular endothelial growth factor (VEGF), TGF, TGFβ, PDGF, IGF, FGF, cytokine, lymphokine, hematopoietic factor, M-CSR, GM-CSF, TNF, interleukin, IL-1, IL-2, IL-3, IL-4, IL-5, IL-6, IL-7, IL-8, IL-9, IL-10, IL-11, IL-12, IL-1 3, IL-14, IL-15, IL-16, IL-17, IL18, IFN, TNF0, TNF1, TNF2, G-CSF, Meg-CSF, GM-CSF, thrombopoietin, stem cell factor, erythropoietin, hepatocyte growth factor/NK1, angiogenic factor, angiopoietin, Ang-1, Ang-2, Ang-4, Ang-Y, human angiopoietin-like polypeptide, angiogenin, morphogenic protein-1, bone morphogenic protein receptor, bone morphogenic protein receptor IA, bone morphogenic protein receptor IB, neurotrophic factor, chemotactic factor, CD proteins, CD3, CD4, CD8, CD19, CD20, erythropoictin, osteoinductive factors, immunotoxin, bone morphogenetic protein (BMP), interferon, interferon-alpha, interferon-beta, interferon-gamma, colony stimulating factor (CSF), M-CSF, GM-CSF, G-CSF, superoxide dismutase, T-cell receptor; surface membrane protein, decay accelerating factor, viral antigen, portion of the AIDS envelope, transport protein, homing receptor, addressin, regulatory protein, integrin, CD11a, CD11b, CD11c, CD18, ICAM, VLA-4, VCAM, tumor associated antigen, HER2, HER3, HER4, nucleophosmin, a heterogeneous nuclear ribonucleoproteins (hnRNPs), fibrillarin; fragments or variants thereof, and any combination thereof.

In still other embodiments, the target biomarker of the one or more aptamer is selected from the group consisting of epidermal growth factor receptor, transferrin receptor, platelet-derived growth factor receptor, Erb-B2, CD 19, CD20, CD45, CD52, Ep-CAM, alpha ([alpha])-fetoprotein, carcinoembryonic antigen peptide-1, caspase-8, CDC27, CDK4, carcino-embryonic antigen, calcium-activated chloride channel-2, cyclophilin B, differentiation antigen melanoma, elongation factor 2, Ephrin type-A receptor 2, 3, Fibroblast growth factor-5, fibronectin, glycoprotein 250, G antigen, N-acetylglucosaminyltransferase V, glycoprotein 100 kD, helicase antigen, human epidermal receptor-2/neurological, heat shock protein 70-2 mutated, human signet ring tumor-2, human telomerase reverse transcriptase, intestinal carboxyl esterase, interleukin 13 receptor [alpha]2 chain, [beta]-D-galactosidase 2-[alpha]-L-fucosyltransferase, melanoma antigen, melanoma antigen recognized by T cells-1/Melanoma antigen A, melanocortin 1 receptor, macrophage colony-stimulating factor, mucin 1, 2, melanoma ubiquitous mutated 1, 2, 3, New York-esophagus 1, ocular albinism type 1 protein, O-linked N-acetyl glucosamine transferase gene, protein 15, promyelocytic leukemia/retinoic acid receptor [alpha], prostate-specific antigen, prostate-specific membrane antigen, receptor-type protein-tyrosinephosphatase kappa, renal antigen, renal ubiquitous 1, 2, sarcoma antigen, squamous antigen rejecting tumor 1, 2, 3, synovial sarcoma, Survivin-2B, synaptotagmin I/synovial sarcoma, X fusion protein, translocation Ets-family leukemia/acute myeloid leukemia 1, transforming growth factor [beta] receptor 2, triosephosphate isomerase, taxol resistant associated protein 3, testin-related gene, tyrosinase related protein 1, tyrosinase related protein 2, and any combination thereof.

The target biomarker of the one or more aptamer can include a cancer-associated or tumor associated biomarker antigen. The cancer-associated antigen may include one or more of human Her2/neu, Her1/EGF receptor (EGFR), HER2 (ERBB2), Her3, Her4, A33 antigen, B7H3, CD5, CD19, CD20, CD22, CD23 (IgE Receptor), C242 antigen, 5T4, IL-6, IL-13, vascular endothelial growth factor VEGF (e.g., VEGF-A), VEGFR-1, VEGFR-2, CD30, CD33, CD37, CD40, CD44, CD51, CD52, CD56, CD74, CD80, CD152, CD200, CD221, CCR4, HLA-DR, CTLA-4, N PC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), integrin αvβ3, integrin α5βt, folate receptor 1, transmembrane glycoprotein NMB, fibroblast activation protein alpha (FAP), glypican 1, glypican 3, glycoprotein 75, TAG-72, MUC1, MUC16 (also known as CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAM F7), EGP40 pancarcinoma antigen, B-cell activating factor (BAFF), platelet-derived growth factor receptor, glycoprotein EpCAM (17-1A), Programmed Death-1 (PD1), Programmed Death Ligand 1 (PD-L1), protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), mesothelin, or any combination thereof. For example, the targeted biomarker can be selected from the group consisting of Her2/neu, Her1/EGFR, TNF-α, B7H3 antigen, CD20, VEGF, CD52, CD33, CTLA-4, tenascin, alpha-4 (α4) integrin, IL-23, amyloid-β, Huntingtin, CD25, nerve growth factor (NGF), TrkA, α-synuclein, and any combination thereof. In some embodiments, the tumor antigen is selected from the group consisting of PSMA, BRCA1, BRCA2, alpha-actinin-4, BCR-ABL fusion protein (b3a2), CASP-8, β-catenin, Cdc27, CDK4, dek-can fusion protein, Elongation factor 2, ETV6-AML1 fusion protein, LDLR-fucosyltransferase AS fusion protein, hsp70-2, KIAAO205, MART2, MUM-1f, MUM-2, MUM-3, neo-PAP, Myosin class I, OS-9g, pml-RAR alpha fusion protein, PTPRK, K-ras, N-ras, CEA, gp100/Pmel17, Kallikrein 4, mammaglobin-A, Melan-A/MART-1, PSA, TRP-1/gp75, TRP-2, tyrosinase, CPSF, EphA3, G250/MN/CAIX, HER-2/neu, Intestinal carboxyl esterase, alpha-fetoprotein, M-CSF, MUC1, p53, PRAME, RAGE-1, RU2AS, survivin, Telomerase, WT1, CA125, and any combination thereof. In still other embodiments, the tumor associated antigen is selected from the group consisting of 4-1BB, 5T4 AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2. EphA3, EphB2, EphB3, FAP, Fibronectin, Folate Receptor, Ganglioside GM3, GD2, glucocorticoid-induced tumor necrosis factor receptor (GITR), gp100, gpA33, GPNMB, ICOS, IGFIR, Integrin av, Integrin αvβ, KIR, LAG-3, Lewis Y, Mesothelin, c-MET, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, variants thereof, and any combination thereof. In still other embodiments, the tumor-associated antigen is selected from the group consisting of Lewis Y, Muc-1, erbB-2, crbB-3, crbB-4, Ep-CAM, EGF-receptor (e.g., EGFR type I or EGFR type II), EGFR deletion neoepitope, CA19-9, Muc-1, LeY, TF-antigen, Tn-antigen, sTn-antigen, TAG-72, PSMA, STEAP, Cora antigen, CD7, CD19, CD20, CD22, CD25, Ig-α, Ig-β, A33, G250, CD30, MCSP, gp100, CD44-v6, MT-MMPs, (MIS) receptor type II, carboanhydrase 9, F19-antigen, Ly6, desmoglein 4, PSCA, Wuc-1, GD2, GD3, TM4SF-antigens (CD63, L6, CO-29, SAS) the alpha and/or gamma subunit of the fetal type acetylcholinreceptor (AChR), and any combination thereof. The cancer antigen can be selected from A33, BAGE, Bcl-2, β-catenin, CA125, CA19-9, CD5, CD19, CD20, CD21, CD22, CD33, CD37, CD45, CD123, CEA, c-Met, CS-1, cyclin B1, DAGE, EBNA, EGFR, ephrinB2, estrogen receptor, FAP, ferritin, folate-binding protein, GAGE, G250, GD-2, GM2, gp75, gp100 (Pmel 17), HER-2/ncu, HPV E6, HPV E7, Ki-67, LRP, mesothelin, p53, PRAME, progesterone receptor, PSA, PSMA, MAGE, MART, mesothelin, MUC, MUM-1-B, myc, NYESO-1, ras, RORI, survivin, tenascin, TSTA tyrosinase, VEGF, WT1, and any combination thereof. In some embodiments, the tumor antigen is selected from carcinoembryonic antigen (CEA), alpha-fetoprotein (AFP), prostate specific antigen (PSA), prostate specific membrane antigen (PSMA), CA-125 (epithelial ovarian cancer), soluble Interleukin-2 (IL-2) receptor, RAGE-1, tyrosinase, MAGE-1, MAGE-2, NY-ESO-1, Melan-A/MART-1, glycoprotein (gp) 75, gp100, beta-catenin, PRAME, MUM-1, ZFP161, Ubiquilin-1, HOX-B6, YB-1, Ostconectin, ILF3, IGF-1, and any combination thereof. In some embodiments, the cancer-related antigen comprises CD2, CD4, CD19, CD20, CD22, CD23, CD30, CD33, CD37, CD40, CD44v6, CD52, CD56, CD70, CD74, CD79a, CD80, CD98, CD138, EGFR (Epidermal growth factor receptor), VEGF (Vascular endothelial growth factor), VEGFRI (Vascular endothelial growth factor receptor I), PDGFR (Platelet-derived growth factor receptor), RANKL (Receptor activator of nuclear factor kappa-B ligand), GPNMB (Transmembrane glycoprotein Neuromedin B), EphA 2 (Ephrin type-A receptor 2), PSMA (Prostate-specific membrane antigen), Cripto (Cryptic family protein 1B), EpCAM (Epithelial cell adhesion molecule), CTLA 4 (Cytotoxic T-Lymphocyte Antigen 4), IGF-IR (Type 1 insulin-like growth factor receptor), GP3 (M13 bacteriophage), GP9 (Glycoprotein IX (platelet), CD42a, GP 40 (Glycoprotein 40kDa), GPC3 (glypican-3), GPC1 (glypican-1), TRAILR1 (Tumor necrosis factor-related apoptosis-inducing ligand receptor 1), TRAILRII (Tumor necrosis factor-related apoptosis-inducing ligand receptor II), FAS (Type II transmembrane protein), PS (phosphatidyl serine) lipid, Gal GalNac Gal N-linked, Muc1 (Mucin 1, cell surface associated, PEM), Muc18, CD146, A5B1 integrin (α5β1), α4β1 integrin, αv integrin (Vitronectin Receptor), Chondrolectin, CAIX (Carbonic anhydrase IX, gene G250/MN-encoded transmembrane protein), GD2 gangloside, GD3 gangloside, GM1 gangloside, Lewis Y, Mesothelin, HER2 (Human Epidermal Growth factor 2), HER3, HER4, FN14 (Fibroblast Growth Factor Inducible 14), CS1 (Cell surface glycoprotein, CD2 subset 1, CRACC, SLAMF7, CD319), 41BB CD137, SIP (Siah-1 Interacting Protein), CTGF (Connective tissue growth factor), HLADR (MHC class II cell surface receptor), PD-1 (Programmed Death 1, Type I membrane protein, PD-L1 (Programmed Death Ligand 1), PD-L2 (Programmed Death Ligand 2), IL-2 (Interleukin-2), IL-8 (Interleukin-8), IL-13 (Interleukin-13), PIGF (Phosphatidylinositol-glycan biosynthesis class F protein), NRP1 (Neuropilin-1), ICAM1, CD54, GC182 (Claudin 18.2), Claudin, HGF (Hepatocyte growth factor), CEA (Carcinoembryonic antigen), LTβR (lymphotoxin β receptor), Kappa Myeloma, Folate Receptor alpha, GRP78 (BIP, 78 kDa Glucose-regulated protein), A33 antigen, PSA (Prostate-specific antigen), CA 125 (Cancer antigen 125 or carbohydrate antigen 125), CA19.9, CA15.3, CA242, leptin, prolactin, osteopontin, IGF-II (Insulin-like growth factor 2), fascin, sPIgR (secreted chain of polymorphic immunoglobulin receptor), 14-3-3 protein eta, 5T4 oncofetal protein, ETA (epithelial tumor antigen), MAGE (Melanoma-associated antigen), MAPG (Melanoma-associated proteoglycan, NG2), vimentin, EPCA-1 (Early prostate cancer antigen-2), TAG-72 (Tumor-associated glycoprotein 72), factor VIII, Neprilysin (Membrane metallo-endopeptidase), 17-1 A (Epithelial cell surface antigen 17-1A), or any combination thereof. The cancer antigen targeted by the one or more aptamer can be selected from the group consisting of carbonic anhydrase IX, alpha-fetoprotein, A3, antigen specific for A33 antibody, Ba 733, BrE3-antigen, CA125, CD1, CDla, CD3, CD5, CD15, CD16, CD19, CD20, CD21, CD22, CD23, CD25, CD30, CD33, CD38, CD45, CD74, CD79a, CD80, CD138, colon-specific antigen-p (CSAp), CEA (CEACAM5), CEACAM6, CSAp, EGFR, EGP-1, EGP-2, Ep-CAM, Flt-1, Flt-3, folate receptor, HLA-DR, human chorionic gonadotropin (HCG) and its subunits, HER2/neu, hypoxia inducible factor (HIF-1), Ia, IL-2, IL-6, IL-8, insulin growth factor-1 (IGF-1), KC4-antigen, KS-1-antigen, KS1-4, Le-Y, macrophage inhibition factor (MIF), MAGE, MUC1, MUC2, MUC3, MUC4, MUC16, NCA66, NCA95, NCA90, antigen specific for PAM-4 antibody, placental growth factor, p53, prostatic acid phosphatase, PSA, PSMA, RS5, S100, TAC, TAG-72, tenascin, TRAIL receptors, Tn antigen, Thomson-Friedenreich antigens, tumor necrosis antigens, VEGF, ED-B fibronectin, 17-1A-antigen, an angiogenesis marker, an oncogene marker, an oncogene product, and any combination thereof.

A tumor biomarker targeted by the one or more aptamer can be a generic tumor marker or be associated with certain tumor types, such as those originating from different anatomical origins. In an embodiment, the tumor marker can be chosen to correspond to a certain tumor type. For example, non-limiting examples of tumor markers and associated tumor types include the following, listed as antigen (optional name; cancer types): Alpha fetoprotein (AFP; germ cell tumor, hepatocellular carcinoma); CA15-3 (breast cancer); CA27-29 (breast cancer); CA19-9 (mainly pancreatic cancer, but also colorectal cancer and other types of gastrointestinal cancer); CA-125 (ovarian cancer, endometrial cancer, fallopian tube cancer, lung cancer, breast cancer and gastrointestinal cancer); Calcitonin (medullary thyroid carcinoma); Calretinin (mesothelioma, sex cord-gonadal stromal tumour, adrenocortical carcinoma, synovial sarcoma); Carcinoembryonic antigen (gastrointestinal cancer, cervix cancer, lung cancer, ovarian cancer, breast cancer, urinary tract cancer); CD34 (hemangiopericytoma/solitary fibrous tumor, plcomorphic lipoma, gastrointestinal stromal tumor, dermatofibrosarcoma protuberans); CD99 (MIC2; Ewing sarcoma, primitive neuroectodermal tumor, hemangiopericytoma/solitary fibrous tumor, synovial sarcoma, lymphoma, leukemia, sex cord-gonadal stromal tumour); CD117 (gastrointestinal stromal tumor, mastocytosis, seminoma); Chromogranin (neuroendocrine tumor); Chromosomes 3, 7, 17, and 9p21 (bladder cancer); Cytokeratin (various types; various carcinoma, some types of sarcoma); Desmin (smooth muscle sarcoma, skeletal muscle sarcoma, endometrial stromal sarcoma); Epithelial membrane antigen (EMA; many types of carcinoma, meningioma, some types of sarcoma); Factor VIII (CD31, FL1; vascular sarcoma); Glial fibrillary acidic protein (GFAP; glioma (astrocytoma, ependymoma)); Gross cystic disease fluid protein (GCDFP-15; breast cancer, ovarian cancer, salivary gland cancer); HMB-45 (melanoma, PEComa (for example angiomyolipoma), clear cell carcinoma, adrenocortical carcinoma); Human chorionic gonadotropin (hCG; gestational trophoblastic disease, germ cell tumor, choriocarcinoma); Immunoglobulin (lymphoma, leukemia); Inhibin (sex cord-gonadal stromal tumour, adrenocortical carcinoma, hemangioblastoma); keratin (various types; carcinoma, some types of sarcoma); lymphocyte marker (various types, lymphoma, leukemia); MART-1 (Melan-A; melanoma, steroid-producing tumors e.g. adrenocortical carcinoma, gonadal tumor); Myo D1 (rhabdomyosarcoma, small, round, blue cell tumour); muscle-specific actin (MSA; myosarcoma (leiomyosarcoma, rhabdomyosarcoma); neurofilament (neuroendocrine tumor, small-cell carcinoma of the lung); neuron-specific enolase (NSE; neuroendocrine tumor, small-cell carcinoma of the lung, breast cancer); placental alkaline phosphatase (PLAP; seminoma, dysgerminoma, embryonal carcinoma); prostate-specific antigen (prostate); PTPRC (CD45; lymphoma, leukemia, histiocytic tumor); S100 protein (melanoma, sarcoma (neurosarcoma, lipoma, chondrosarcoma), astrocytoma, gastrointestinal stromal tumor, salivary gland cancer, some types of adenocarcinoma, histiocytic tumor (dendritic cell, macrophage)); smooth muscle actin (SMA; gastrointestinal stromal tumor, leiomyosarcoma, PEComa); synaptophysin (neuroendocrine tumor); thyroglobulin (thyroid cancer but not typically medullary thyroid cancer); thyroid transcription factor-1 (all types of thyroid cancer, lung cancer); Tumor M2-PK (colorectal cancer, Breast cancer, renal cell carcinoma, Lung cancer, Pancreatic cancer, Esophageal Cancer, Stomach Cancer, Cervical Cancer, Ovarian Cancer); Vimentin (sarcoma, renal cell carcinoma, endometrial cancer, lung carcinoma, lymphoma, leukemia, melanoma). Additional tumor types and associated biomarkers which may be targeted by the one or more aptamer comprise the following, listed as tumor type (markers): Colorectal (M2-PK, CEA, CA 19-9, CA 125); Breast (CEA, CA 15-3, Cyfra 21-1); Ovary (CEA, CA 19-9, CA 125, AFP, BHCG); Uterine (CEA, CA 19-9, CA 125, Cyfra 21-1, SCC); Prostate (PSA); Testicle (AFP, BHCG); Pancreas/Stomach (CEA, CA 19-9, CA 72-4); Liver (CEA, AFP); Oesophagus (CEA, Cyfra 21-1); Thyroid (CEA, NSE); Lung (CEA, CA 19-9, CA 125, NSE, Cyfra 21-1); Bladder (CEA, Cyfra 21-1, TPA). One or more of these markers can be used as the target biomarker recognized by the aptamer of the construct of the invention.

In some embodiments of the invention, the target biomarker recognized by the one or more aptamer comprises PDGF, IgE, IgE Fce R1, PSMA, CD22, TNF-alpha, CTLA4, PD-1, PD-L1, PD-L2, FcRIIB, BTLA, TIM-3, CD11c, BAFF, B7-X, CD19, CD20, CD25, CD33, and any combination thereof. The target biomarker can also be a protein comprising insulin-like growth factor 1 receptor (IGFIR), IGF2R, insulin-like growth factor (IGF), mesenchymal epithelial transition factor receptor (c-met), hepatocyte growth factor (HGF), epidermal growth factor receptor (EGFR), ErbB2, ErbB3, epidermal growth factor (EGF), heregulin, fibroblast growth factor receptor (FGFR), platelet-derived growth factor receptor (PDGFR), platelet-derived growth factor (PDGF), vascular endothelial growth factor receptor (VEGFR), vascular endothelial growth factor (VEGF), tumor necrosis factor receptor (TNFR), tumor necrosis factor alpha (TNF-a), folate receptor (FOLR), folate, transferrin receptor (TfR), mesothelia, Fc receptor, c-kit receptor, c-kit, a4 integrin, P-selectin, sphingosine-1-phosphate receptor-1 (SIPR), hyaluronate receptor, leukocyte function antigen-1 (LFA-1), CD4, CD11, CD18, CD20, CD25, CD27, CD52, CD70, CD80, CD85, CD95 (Fas receptor), CD106 (vascular cell adhesion molecule 1 (VCAM1)), CD166 (activated leukocyte cell adhesion molecule (ALCAM)), CD 178 (Fas ligand), CD253 (TNF-related apoptosis-inducing ligand (TRAIL)), inducible costimulator (ICOS) ligand, CCR2, CXCR3, CCR5, CXCL12 (stromal cell-derived factor 1 (SDF-1)), interleukin 1 (IL-1), cytotoxic T-lymphocyte antigen 4 (CTLA-4), MART-1, gp100, MAGE-1, ephrin (Eph) receptor, mucosal addressin cell adhesion molecule 1 (MAdCAM-1), carcinoembryonic antigen (CEA), LewisY, MUC-1, epithelial cell adhesion molecule (EpCAM), cancer antigen 125 (CA125), prostate specific membrane antigen (PSMA), TAG-72 antigen, fragments thereof, and any combination thereof. In various embodiments, the target biomarker of the one or more aptamer comprises one or more of PSMA, PSCA, e selectin, an ephrin, ephB2, cripto-1, TENB2 (TEMFF2), ERBB2 receptor (HER2), MUC1, CD44v6, CD6, CD19, CD20, CD22, CD23, CD25, CD30, CD33, CD56, IL-2 receptor, HLA-DR10 B subunit, EGFR, CA9, caveolin-1, nucleolin, and any combination thereof.

Any useful combination of cancer antigens, tumor antigens, tissue antigens and microvesicle antigens, such as those above, can be targeting by the construct of the invention. For example, aptamers to multiple targets may be incorporated into a nanoparticle construct of the invention. As novel cancer biomarkers are discovered, the SELEX process or some modification thereof can be used to identify an aptamer to such target and therefor target the novel biomarker.

One of skill will appreciate that the assembly of the invention may be used to deliver any appropriate payload to any target cell.

By way of a non-limiting example, the aptamer may target the hepatocyte growth factor receptor (HGFR), also called cMet. HGFR is a transmembrane receptor protein that is overexpressed on the surface of numerous solid tumors. The ability to bind extracellular cMet by the aptamer moieties is a further feature supporting efficient uptake into cancer cells. In an embodiment, the anti-cMet aptamer comprises the nucleotide sequence 5′-TGGATGGTAGCTCGGTCGGGGTGGGTGGGTTGGCAAGTCT-3′ (SEQ ID NO. 1). Aptamers comprising the SEQ ID NO. 1 bind with high specificity and affinity to the hepatocyte growth factor receptor, particularly with nano molar affinity. The aptamer may comprise a functional variant of SEQ ID NO. 1. A “functional variant” means that the sequence comprises one or more modification but retains the ability to bind its target with sufficient specificity and affinity. Such modification can include modified bases, deletions, insertions, and the like. A lipid-modified anti-cMet aptamer, e.g., comprising the sequence SEQ ID NO. 1, may contain four C₁₂-lipid-functionalized dU-phosphoramidites at the 5′-end. It was found that lipidation of a cMet-binding aptamer improves efficient uptake into cancer cells. See, e.g., Example 8 herein. Without being bound by theory, efficient uptake into cancer cells may be due to the ability of the aptamer to bind extracellular cMet, and the ability to form nanoconstructs due to the lipidation.

As described herein, the assembly of the invention may comprise a moiety that can capture and release a drug upon a given condition. In a preferred embodiment, the nucleic acid-based assembly comprises at least one nucleic acid motif designed to physically capture a drug. In some embodiments, the nucleic acid motif is a 5′-GC rich oligodeoxynucleotide that forms one or more hairpin loops. Such loops structure can be configured to intercalate the drug. Such 5′-GC-rich hairpin oligodeoxynucleotide can intercalate and transport planar aromatic therapeutic agents such as doxorubicin. See, e.g., Examples 9-10 herein. The nucleic acid motif may contain several GC rich hairpins, for example 2, 3, 4, 5, 6, 7, 8, 9, 10, or more than 10, GC rich hairpins. In preferred embodiments, the motif contains three or four GC rich hairpins. Integrating multiple GC-rich hairpin-duplex motifs affords several folds of loading of drug into a single nano scaffold, thereby enhancing the payload capacity in comparison to a monomeric aptamer.

The nucleic acid motif may comprise one or more moieties that effect the release of the drug under certain conditions. For example, external stimuli such as temperature, irradiation, or environmental stimuli, such as pH or other stimulants may initiate release of the drug. In preferred embodiments, the nucleic acid motif comprises at least one photo-responsive moiety located within the base-pairing regions into which the drug intercalates, particularly within the hairpin region or regions. As used herein, the term “photo-responsive” moiety refers to an organic group, which undergoes isomerization and conformational change induced by irradiation, for example with visible light, ultraviolet light, or X-ray. One such photo-responsive moiety is an azobenzene group, a molecule with two phenyl rings joined by an azo linkage. Azobenzene can reversibly change trans/cis conformation upon exposure to irradiation energy. The photo induced transformation of photo-responsive molecules such as azobenzene derivatives incorporated into oligodeoxynucleotide backbones leads to a molecular motion which causes a structural change and thus is able to reversibly open and close oligodeoxynucleotide duplexes upon irradiation. Preferred azobenzene derivatives include 2′-methylazobenzene, and particularly 2′,6′-dimethylazobenzene (DMAB). The motif may contain any number of appropriate photo-responsive molecules. In some embodiments, the nucleic acid motif contains several such moieties, for example 1 to 10, 2 to 6, or preferably 3, 4 or 5, dimethylazobenzene moieties.

As a non-limiting example, azobenzenes tethered on D-threoninol can allow incorporation of the azobenzenes into oligodeoxynucleotide backbones. The nucleic acid motif may contain one or more, for example 1 to 10, 2 to 6, preferably 3, 4 or 5, particularly four, 2′,6′-dimethylazobenzene-D-threoninol residues. In some embodiments, the nucleic acid motif comprises the nucleotide sequence 5′-GCNGCGNCTCNGCGNCGATTATTACGCGCGAGCGCGC-3′ (SEQ ID NO: 2) or a functional variant thereof. In this context. “functional variant” means that the sequence comprises one or more modification such as described herein but retains the ability to effect release, e.g., change conformation, upon external stimuli. The N can be 2′-methylazobenzene modified, including without limitation a 2′,6′-dimethylazobenzenc-D-threoninol residue. The assembly thus can advantageously be provided with a built-in photo-regulated release mechanism for the drug. In a preferred embodiment, the nucleic acid motif comprises the sequence SEQ ID NO: 2 with 4 DMAB moieties introduced into the sequence, and four lipid-chains attached to the 5′-end.

As used herein, the term “drug” refers to any substance, other than food, that causes a physiological change in the body. The drug incorporated into the assembly of the invention may comprise a regulatory molecule, such as an antagomir, small interfering RNA, microRNA, pharmaceutical drug, or any combination thereof. In certain embodiments, the drug is an anti-cancer drug. As a non-limiting example, the drug can be doxorubicin (DxR), a potent and widely used chemotherapeutic. The IUPAC name of doxorubicin is (7S,9S)-7-[(2R,4S,5S,6S)-4-amino-5-hydroxy-6-methyloxan-2-yl]oxy-6,9,11-trihydroxy-9-(2-hydroxyacetyl)-4- methoxy-8,10-dihydro-7H-tetracene-5,12-dione. Doxorubicin is a planar aromatic molecule that is able to intercalate into oligodeoxynucleotides such as SEQ ID NO: 2.

The invention contemplates the delivery of any useful and appropriate drug, including drug cocktails and combination therapy. In embodiments of the invention, the drug may include, without limitation, one or more of Abemaciclib, Abiraterone Acetate, Abitrexate (Methotrexate), ABVD (Doxorubicin Hydrochloride (Adriamycin), Bleomycin, Vinblastine Sulfate, Dacarbazine), ABVE (Doxorubicin Hydrochloride (Adriamycin), Bleomycin, Vinblastine Sulfate, Etoposide Phosphate), ABVE-PC (Doxorubicin Hydrochloride (Adriamycin), Bleomycin, Vinblastine Sulfate, Etoposide Phosphate, Prednisone, Cyclophosphamide), AC (Doxorubicin Hydrochloride (Adriamycin), Cyclophosphamide, Acalabrutinib, AC-T (Doxorubicin Hydrochloride (Adriamycin), Cyclophosphamide, Paclitaxel (Taxol)), Adcetris (Brentuximab Vedotin), ADE (Cytarabine (Ara-C), Daunorubicin Hydrochloride, Etoposide Phosphate), Ado-Trastuzumab Emtansine, Adriamycin (Doxorubicin Hydrochloride), Afatinib Dimaleate, Afinitor (Everolimus), Akynzeo (Nctupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran (Melphalan; Melphalan Hydrochloride), Aloxi (Palonosetron Hydrochloride), Alunbrig (Brigatinib), Ambochlorin (Chlorambucil), Amboclorin (Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axicabtagene Cilolcucel, Axitinib, Azacitidine, Bavencio (Avelumab), BEACOPP, Becenum (Carmustine), Beleodaq (Belinostat), Belinostat, Bendamustine Hydrochloride, BEP (Bleomycin, Etoposide Phosphate, Cisplatin (Platinol)), Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel (Busulfan, Melphalan Hydrochloride). Busulfan, Busulfox (Busulfan), Cabazitaxel, Cabomctyx (Cabozantinib-S-Malate), Cabozantinib-S-Malate, CAF (Cyclophosphamide, Doxorubicin Hydrochloride (Adriamycin), Fluorouracil), Calquence (Acalabrutinib), Campath (Alemtuzumab), Camptosar (Irinotecan Hydrochloride), Capecitabine, CAPOX (Capecitabine, Oxaliplatin), Carboplatin, CARBOPLATIN-TAXOL, Carfilzomib, Carmubris (Carmustine), Carmustine, Casodex (Bicalutamide), CEM (Carboplatin, Etoposide Phosphate, Melphalan Hydrochloride), Ceritinib, Cerubidine (Daunorubicin Hydrochloride), Cetuximab, CEV (Carboplatin, Etoposide Phosphate, Vincristine Sulfate), Chlorambucil, CHLORAMBUCIL-PREDNISONE, CHOP (Cyclophosphamide, Doxorubicin Hydrochloride (Hydroxydaunomycin), Vincristine Sulfate (Oncovin), Prednisone), Cisplatin, Cladribine, Clafen (Cyclophosphamide), Clofarabine, Clofarex (Clofarabine), Clolar (Clofarabine), CMF (Cyclophosphamide, Methotrexate, Fluorouracil), Cobimetinib, Cometriq (Cabozantinib-S-Malate), Copanlisib Hydrochloride, COPDAC (Cyclophosphamide, Vincristine Sulfate (Oncovin), Prednisone, Dacarbazine), COPP (Cyclophosphamide, Vincristine Sulfate (Oncovin), Procarbazine Hydrochloride, Prednisone), COPP-ABV (Cyclophosphamide, Vincristine Sulfate (Oncovin), Procarbazine Hydrochloride, Prednisone, Doxorubicin Hydrochloride (Adriamycin), Bleomycin. Vinblastine Sulfate), Cosmegen (Dactinomycin), Cotellic (Cobimetinib), Crizotinib, CVP (Cyclophosphamide, Vincristine Sulfate, Prednisone), Cyclophosphamide, Cyfos (Ifosfamide), Cyramza (Ramucirumab), Cytarabinc, Cytosar-U (Cytarabinc), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Decitabine, Defibrotide Sodium, Defitelio (Defibrotide Sodium), Degarclix, Denilcukin Diftitox, Denosumab, Dexamethasone, Dexrazoxane Hydrochloride, Dinutuximab, Docetaxel, Doxorubicin Hydrochloride, DTIC-Dome (Dacarbazine), Durvalumab, Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH (Etoposide Phosphate, Prednisone, Vincristine Sulfate (Oncovin), Cyclophosphamide, Doxorubicin Hydrochloride (Hydroxydaunomycin)), Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Everolimus, Evista (Raloxifene Hydrochloride), Evomela (Melphalan Hydrochloride), Exemestane, 5-FU (Fluorouracil), Fareston (Toremifene), Farydak (Panobinostat), Faslodex (Fulvestrant), FEC, Femara (Letrozole), Filgrastim, Fludara (Fludarabine Phosphate), Fludarabine Phosphate, Flutamide, Folex (Methotrexate), Folex PFS (Methotrexate), FOLFIRI (Leucovorin Calcium (Folinic Acid), Fluorouracil, Irinotecan Hydrochloride), FOLFIRI-BEVACIZUMAB, FOLFIRI-CETUXIMAB, FOLFIRINOX (Leucovorin Calcium (Folinic Acid), Fluorouracil, Irinotecan Hydrochloride, Oxaliplatin), FOLFOX (Leucovorin Calcium (Folinic Acid), Fluorouracil, Oxaliplatin), Folotyn (Pralatrexate), FU-LV (Fluorouracil, Leucovorin Calcium), Fulvestrant, Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Glucarpidase, Goserelin Acetate, Halaven (Eribulin Mesylate), Hemangeol (Propranolol Hydrochloride), Herceptin (Trastuzumab), Hycamtin (Topotecan Hydrochloride), Hydrea (Hydroxyurea), Hydroxyurea, Hyper-CVAD (Cyclophosphamide, Vincristine Sulfate, Doxorubicin Hydrochloride (Adriamycin), Dexamethasone), Ibrance (Palbociclib), Ibritumomab Tiuxetan, Ibrutinib, ICE (Ifosfamide, Carboplatin, Etoposide Phosphate), Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant. Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Istodax (Romidepsin), Ixabepilone, Ixazomib Citrate, Ixempra (Ixabepilone), Jakafi (Ruxolitinib Phosphate). JEB (Carboplatin (JM8), Etoposide Phosphate, Bleomycin), Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate. Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP (Mechlorethamine Hydrochloride, Vincristine Sulfate (Oncovin), Procarbazine Hydrochloride, Prednisone), Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA (Vincristine Sulfate (Oncovin), Etoposide Phosphate, Prednisone, Doxorubicin Hydrochloride (Adriamycin)), Ofatumumab, OFF (Oxaliplatin, Fluorouracil, Leucovorin Calcium (Folinic Acid)), Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA (Vincristine Sulfate (Oncovin), Procarbazine Hydrochloride, Prednisone, Doxorubicin Hydrochloride (Adriamycin)), Osimertinib, Oxaliplatin, Paclitaxel, Paclitaxel Albumin-stabilized Nanoparticle Formulation, PAD (Bortezomib (PS-341), Doxorubicin Hydrochloride (Adriamycin), Dexamethasone), Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV (Procarbazine Hydrochloride, Lomustine (CCNU), Vincristine Sulfate), PEB (Cisplatin (Platinol), Etoposide Phosphate, Bleomycin), Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP (Rituximab+CHOP), R-CVP (Rituximab+CVP), Recombinant Interferon Alfa-2b, Regorafenib, Relistor (Methylnaltrexone Bromide), R-EPOCH (Rituximab+EPOCH), Revlimid (Lenalidomide), Rheumatrex (Methotrexate), Ribociclib, R-ICE (Rituximab+ICE), Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Siltuximab, Sipuleucel-T, Somatuline Depot (Lanreotide Acetate), Sonidegib, Sorafenib Tosylate, Sprycel (Dasatinib), STANFORD V (Mechlorethaminc Hydrochloride, Doxorubicin Hydrochloride. Vinblastine Sulfate, Vincristine Sulfate, Bleomycin, Etoposide Phosphate, Prednisone), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC (Docetaxel (Taxotere), Doxorubicin Hydrochloride (Adriamycin), Cyclophosphamide), Tafinlar (Dabrafenib), Tagrisso (Osimertinib), Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tisagenlecleucel, Topotecan Hydrochloride, Toremifene, Torisel (Temsirolimus), Tositumomab and Iodine I 131 Tositumomab, Totect (Dexrazoxane Hydrochloride), TPF (Docetaxel (Taxotere), Cisplatin (Platinol), Fluorouracil), Trabectedin, Tramctinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC (Vincristine Sulfate, Dactinomycin (Actinomycin-D), Cyclophosphamide), Valrubicin, Valstar (Valrubicin), Vandetanib, VAMP (Vincristine Sulfate, Doxorubicin Hydrochloride (Adriamycin), Methotrexate, Prednisone), Varubi (Rolapitant Hydrochloride), Vectibix (Panitumumab), VeIP (Vinblastine Sulfate (Velban), Ifosfamide, Cisplatin (Platinol)), Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP (Etoposide Phosphate (VePesid), Ifosfamide, Cisplatin (Platinol)), Vismodegib, Vistogard (Uridine Triacetate), Voraxaze (Glucarpidasc), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI (Capecitabine (Xeloda), Irinotecan Hydrochloride), XELOX (Capecitabine (Xeloda), Oxaliplatin), Xgeva (Denosumab), Xofigo (Radium 223 Dichloride), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinccard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate). Targeted delivery of the drug may allow use of drugs conventionally associated with treating certain cancers to treat other types of cancer.

In embodiments, the nucleic acid-based assembly may comprise any desired number of aptamers to different target proteins. As a non-limiting example, consider that an assembly comprises a second aptamer, particularly a second nucleic acid aptamer. A “second aptamer” as used herein refers to a second species of aptamer and is not intended to limit the number of aptamer molecules comprised in the assembly. A preferred second aptamer is an aptamer targeting a different target than the “first” aptamer, e.g., a different cancer biomarker protein, or a protein that is (over) expressed on a target cell such as a cancer cell. Useful biomarker target proteins are described herein or can be selected as their use becomes apparent. In some cases, the aptamers with the assembly of the invention are selected against desired cellular targets, e.g., cancer cells, such that the precise target biomolecule is unknown.

The drug can be released from the nucleic acid-based assembly by various stimuli. In embodiments, the drug is released upon irradiation. The irradiation may comprise visible light, ultraviolet light, or X-ray. Visible light may have a wavelength in a range from 380 nm to 435 nm. Visible light may cause no or only limited harm to the irradiated tissue. Suitable UV light irradiation may have a wavelength in a range from 320 nm to 400 nm. For example, one usable UV wavelength is 365 nm. As an illustration, we found that UV irradiation lead to release of most an intercalated drug from an assembly of the invention followed by transfer of the drug to the cell nuclei. See, e.g., Example 9 herein. UV light triggering with a penetration depth of light of a few millimeters may be sufficient for use with some melanoma. For other cancer types, azobenzene photo switches that isomerize with red light may be preferred. Alternatively, fiber optic endoscopy might direct UV light to potential tumor sites deeper inside the body. Suitable X ray irradiation may have a wavelength in a range from 630 nm to 660 nm. X ray irradiation may not only release the drug, but also itself have a therapeutic effect on the cancer cells. The invention contemplates any useful means of stimulating drug release.

The lipid-mediated facile assembly of the aptamer and nucleic acid motifs into hybrid nano-constructs further advantageously allows for a precise control of the aptamer density on the surface of the assembly. Such density can be controlled by mixing the cell-targeting aptamer with the drug-carrying nucleic acid motif in different ratios. In embodiments, the lipid-modified aptamer and nucleic acid motif are present in the assembly in a ratio in a range from ≥1:10 to ≤10:1, such as ≥1:5 to ≤5:1. or ≥1:2 to ≤3:2. In embodiments, the lipid-modified aptamer and nucleic acid motif are present in a 1:1 ratio. Such ratios can provide for an assembly providing most advantageous balance between high target affinity and internalization efficiency and therapeutically effective results based on drug carrying efficiency.

The assembly can be prepared by mixing the lipid-modified aptamer and nucleic acid motif at the desired ratio (e.g., ≥1:2 to ≤3:2. or 1:1) with the drug. Preferably the drug is used in excess, for example at 2-fold, 3-fold, 4-fold, 5-fold, 6-fold, 7-fold, 8-fold, 9-fold, or 10-fold excess. As desired, the drug is used in greater than 10-fold excess in the assembly. The ratio may be determined depending on the nature of the drug itself, e.g., potency, structure, size, etc. The forming of the hybrid nanoconstruct may be followed by a purification step, including without limitation chromatography or filtration techniques. In some embodiments, the filtration comprises spin filtration using a centrifugal filter. See, e.g., Example 1 herein. The purification may be used to remove unencapsulted drug and the like.

In various embodiments, the nucleic acid-based assembly of the invention can exploit aptamer-mediated selective cell targeting, photo induced structure switching, and lipid-mediated self-assembly, and thus provide a hybrid assembly as a molecular carrier system that allows selective transport of intercalated cytotoxic drugs to target cells and release of the payload under light irradiation. This design offers the possibility to self-assemble multiple functional domains at once into a single nanoconstruct, such as the targeting ability of one or more aptamer, and an intercalated drug-carrying motif, compared to the limited possibility of introducing multiple functionalities into a single modified aptamer system through inherent synthetic efforts. Moreover, multiple aptamer motifs that target different biomarkers on the cell surface may be assembled in a single nanoparticle by mixing the respective lipidated aptamers to allow for a more precise targeting.

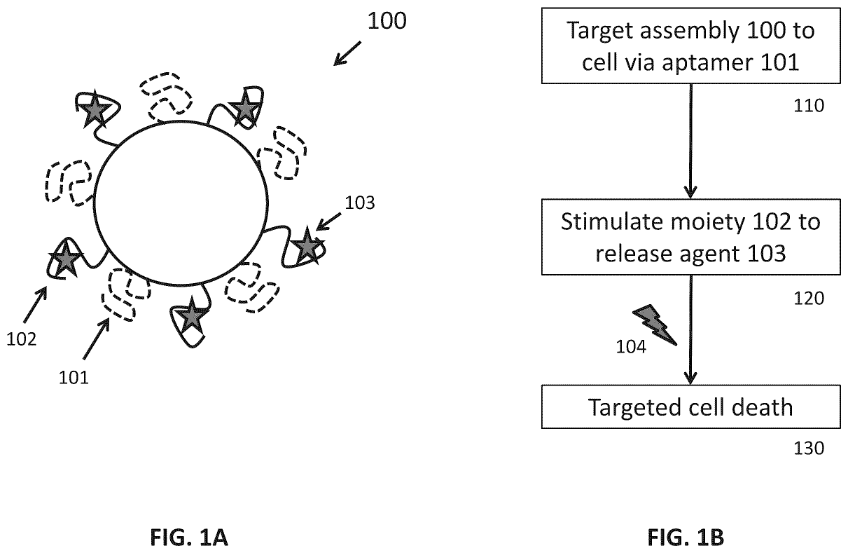

A further aspect of the present invention relates to a nucleic acid-based assembly according to the invention for use as a medicament. The medicament may be used for treating diseases and disorders, e.g., any diseases and disorders that may be treated by delivery of a compound such as a drug. In preferred embodiments, the medicament is used in the treatment of cancer. See FIGS. 1A-B for an illustration of the use of the medicament of the invention. In this example, a lipid-functionalized nucleic acid-based assembly 100 comprises a cell-targeting aptamer 101 accompanied by a photo-responsive oligonucleotide motif 102 that can selectively target and transport high doses of pharmaceutically active molecules 103. including without limitation such drugs as described herein. In step 110, the assembly 100 is contacted with the cells targeted by aptamer 101. The assembly may be internalized by the cell as described herein. In step 120, the construct is stimulated to release payload 103, e.g., by irradiation 104. The payload 103 is then able to exert its influence over the target cell, e.g., by causing cellular death (step 130). The assembly is advantageous for aptamer-based targeted therapeutics, from fabrication of nanoconstructs of improved scrum stability to efficient cell internalization, and light-triggered release of active therapeutics. In addition, the stability of the nanocontruct and improved cell internalization can be provided by lipidation. In the Examples, we demonstrate using the targeting ability of an anti-cMet aptamer to effect selective transport of a nonconstruct comprising the drug doxibubicin into targeted cancer cells. We show highly efficient cell-uptake of the hybrid-aptameric nanoconstruct into cancer cells, as well as an improved effect on tumor cells by stimulating release of the anti cancer drug inside the cells using a light trigger. See, e.g., Example 10 herein.

The nucleic acid-based assemblies are useful for targeting a variety of cancers, including without limitation solid tumors. As used herein, the term “solid tumor” refers to a solid mass of cancer cells that grow in organ systems and can occur anywhere in the body. In embodiments, the solid tumors are selected from the group comprising breast cancer, prostate cancer, colorectal cancer, ovarian cancer, thyroid cancer, lung cancer, liver cancer, pancreatic cancer, gastric cancer, melanoma (skin cancer), lymphoma and glioma.

A cancer targeted by the assembly of the invention can comprise, without limitation, a carcinoma, a sarcoma, a lymphoma or leukemia, a germ cell tumor, a blastoma, or other cancers. Carcinomas include without limitation epithelial neoplasms, squamous cell neoplasms squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, hurthle cell adenoma, renal cell carcinoma, grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonci, ductal, lobular and medullary neoplasms, acinar cell neoplasms, complex epithelial neoplasms, warthin's tumor, thymoma, specialized gonadal neoplasms, sex cord stromal tumor, thecoma, granulosa cell tumor, arrhenoblastoma, sertoli leydig cell tumor, glomus tumors, paraganglioma, phcochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. Sarcoma includes without limitation Askin's tumor, botryodies, chondrosarcoma, Ewing's sarcoma, malignant hemangio endothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovialsarcoma. Lymphoma and leukemia include without limitation chronic lymphocytic leukemia/small lymphocytic lymphoma, B-cell prolymphocytic leukemia, lymphoplasmacytic lymphoma (such as waldenström macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical hodgkin lymphomas (nodular sclerosis, mixed cellularity, lymphocyte-rich, lymphocyte depleted or not depleted), and nodular lymphocyte-predominant hodgkin lymphoma. Germ cell tumors include without limitation germinoma, dysgerminoma, seminoma, nongerminomatous germ cell tumor, embryonal carcinoma, endodermal sinus turmor, choriocarcinoma, teratoma, polyembryoma, and gonadoblastoma. Blastoma includes without limitation nephroblastoma, medulloblastoma, and retinoblastoma. Other cancers include without limitation labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngcoma, osteosarcoma, chondrosarcoma, myosarcoma, liposarcoma, fibrosarcoma, Ewing sarcoma, and plasmocytoma.

In a further embodiment, the cancer may be a lung cancer including non-small cell lung cancer and small cell lung cancer (including small cell carcinoma (oat cell cancer), mixed small cell/large cell carcinoma, and combined small cell carcinoma), colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemia, lymphoma, myeloma, or other solid tumor.

In embodiments, the cancer comprises an acute lymphoblastic leukemia; acute myeloid leukemia; adrenocortical carcinoma; AIDS-related cancer; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medullocpithelioma, pineal parenchymal tumors of intermediate differentiation, supratentorial primitive neuroectodermal tumors and pincoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medullocpithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; mycloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pincoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sézary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer: Waldenström macroglobulinemia: or Wilm's tumor. The methods of the invention can be used to target these and other cancers.

In some embodiments, the cancer comprises an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumors (GIST), glioblastoma, head and neck squamous carcinoma, leukemia, liver hepatocellular carcinoma, low grade glioma, lung bronchioloalveolar carcinoma (BAC), lung non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma. The assemblies of the invention can be used to target these and other cancers.

It will be appreciated that a single construct may be used to target multiple cancers by selection of aptamers to appropriate biomarkers. As non-limiting examples, consider an aptamer to the cancer antigen HER2. A construct with such an aptamer could be used to target any tumor expressing HER2, such as breast, ovarian, gastric or colorectal cancers. See Liu Z et al., Novel HER2 aptamer selectively delivers cytotoxic drug to HER2-positive breast cancer cells in vitro, J Transl Med. 2012 Jul. 20; 10:148; Takegawa and Yoncsaka, HER2 as an Emerging Oncotarget for Colorectal Cancer Treatment After Failure of Anti-Epidermal Growth Factor Receptor Therapy. Clin Colorectal Cancer. 2017 December; 16(4):247-251; which references are incorporated by reference herein in their entirety. As another example, cMET is expressed in a number of solid tumors, including brain, breast, ovarian, cervical, colorectal, gastric, head and neck, lung (including non-small-cell lung cancer (NSCLC)), liver, skin, prostate and soft tissue cancers. Thus, a construct with an anti-cMET aptamer such as exemplified herein could be used to treat multiple cancer types such as these. See, e.g., Blumenschein GR Jr et al., Targeting the hepatocyte growth factor-cMET axis in cancer therapy. J Clin Oncol. 2012 Sep. 10; 30(26):3287-96; Kim and Kim, Progress of antibody-based inhibitors of the HGF-cMET axis in cancer therapy, Exp Mol Med. 2017 March; 49(3): c307; which references are incorporated by reference herein in their entirety.

For use as a medicament, the nucleic acid-based assembly can be used or included in a composition. Accordingly, in another aspect the present invention relates to a pharmaceutical composition comprising as an active ingredient a nucleic acid-based assembly according to the invention. The pharmaceutical composition is suitable for use in the treatment of cancer, e.g., in the treatment of solid tumors, by choosing appropriate aptamer targeting moities. The nucleic acid-based assembly can be dissolved or dispersed in a pharmaceutically acceptable carrier. The term “pharmaceutical or pharmacologically acceptable” refers to molecular entities and compositions that do not produce an adverse, allergic or other untoward reaction when administered to a subject, such as, for example, a human, as appropriate. The pharmaceutical carrier can be, for example, a solid, liquid, or gas. Suitable carriers and adjuvants can be solid or liquid and correspond to the substances ordinarily employed in formulation technology for pharmaceutical formulations. For compositions convenient pharmaceutical media may be employed. For example, water, buffers, and the like may be used to form liquid preparations such as solutions. Non-limiting examples of formulations that may be useful for the medicament of the invention can be found in Arias JL. Liposomes in drug delivery: a patent review (2007—present). Expert Opin Ther Pat. 2013 November; 23 (11):1399-414; Pérez-Herrero E and Fernández-Medarde A, Advanced targeted therapies in cancer: Drug nanocarriers, the future of chemotherapy. Eur J Pharm Biopharm. 2015 June; 93:52-79; Bulbake U, et al., Liposomal Formulations in Clinical Use: An Updated Review. Pharmaceutics. 2017 Mar. 27; 9 (2); which references are incorporated by reference herein in their entirety.

The present invention also relates to the use of a nucleic acid-based assembly according to the invention for the manufacture of a medicament useful for the treatment of diseases or disorders. Such diseases or disorders include without limitation various cancers as described herein.

In a related aspect, the present invention provides a method of treating a disease or disorder, for example a cancer, including without limitation solid tumors. The method comprises the step of administering to a subject in need thereof a therapeutically effective amount of a medicament comprising a nucleic acid-based assembly according to the invention. Subjects include both human subjects and animal subjects, particularly mammalian subjects such as human subjects or mice or rats for medical purposes. The term “therapeutically effective amount” is used herein to mean an amount or dose sufficient to cause a therapeutic benefit such as an improvement in a clinically significant condition in the subject. A therapeutically effective amount includes but it not limited an amount or dose sufficient to cause to remission or cure.

In some embodiments, the cancer comprises a solid tumor. Solid tumors include without limitation breast cancer, prostate cancer, colorectal cancer, ovarian cancer, thyroid cancer, lung cancer, liver cancer, pancreatic cancer, gastric cancer, melanoma (skin cancer), lymphoma, or glioma. Other contemplated cancers are described above.

By exploiting aptamer-mediated selective cell targeting, photoinduced structure switching, and lipid-mediated self-assembly, the invention provides a hybrid aptamer-nanoconstruct as a molecular carrier system that allows selective transport of intercalated cytotoxic drugs to target cells and release of the payload under light irradiation. See, e.g., FIGS. 1A-B. This design offers the possibility to self-assemble multiple functional domains at once into a single nanoconstruct, as demonstrated herein using the targeting ability an aptamer and an intercalated drug-carrying motif, compared to the limited possibility of introducing multiple functionalities into a single modified aptamer system through inherent synthetic efforts. See Examples 1-10 herein. Fluorescence studies with pyrene loading showed that the self-aggregated nanoconstructs were stabilized in aqueous solution through hydrophobic interaction of the lipids. The mixed nature of the nanoconstructs and their size was confirmed by FRET studies and AFM measurements. Indeed, such self-assembled structures even offer an unprecedented degree of control over the ratio of different functional domains based on the therapeutic requirements. Moreover, integrating multiple GC-rich hairpin-duplex motifs affords several folds of loading of DxR into a single nanoscaffold, thereby enhancing the payload capacity in comparison to a monomeric aptamer.

Confocal imaging and cell-viability assays further demonstrated a highly efficient cell-uptake of the designed hybrid-aptameric nanoconstruct into H1838 cells and an improved effect on tumor cell targeting by releasing DxR inside the cell by a light trigger. The skin depth UV light may be advantageous for some applications, such as treating melanoma. Potential risks associated with UV light such as cellular damage and stability of biological systems may be avoided by using low intensity irradiation for a short period of time as indicated by our experiments. Alternate choices include azobenzene photoswitches that isomerize with red light that has significantly higher skin penetration depth. As another alternative, fiber optic endoscopy might direct UV light to potential tumor sites deeper inside the body.

The invention provides a stable nanoconstruct with high resistance against nucleases accompanied by a greatly improved cell-uptake compared to the unmodified aptamer. The nanoconstructs can be modified to alter characteristics as desired. For example, stability may be controlled with longer lipid tails to the oligonucleotide motifs, or using unsaturated lipids and cross-linking them inside the lipid core.

The invention addresses fundamental obstacles related to aptamer-mediated tumor targeting while designing a multifunctional nanoconstruct with improved nuclease stability; high target binding affinity; and increased tumor uptake, essential prerequisites for next generation aptamer-based targeted therapeutics. Taken together all these combined features make this platform widely applicable for the delivery of a variety of different regulatory molecules, such as AntagomiRs, small interfering RNAs, microRNAs, drugs, and other molecules with high specificity and efficiency to specifically block functions of disease-relevant biomolecules.

Unless otherwise defined, the technical and scientific terms used herein have the same meaning as commonly understood by one of ordinary skill in the art to which this invention belongs.

The examples that follow serve to illustrate the invention in more detail but do not constitute a limitation thereof.

### EXAMPLES

### Example 1: Materials and Methods

### 1.1 Materials

All chemicals including doxorubicin (DxR) were purchased from Sigma-Aldrich unless otherwise specified and were used as received. cMet-Fc, which represents the ectodomain of cMet fused to the Fc domain of human IgG1 was purchased from R&D Systems. Wheat Germ Agglutinin, Alexa Fluor® 488 Conjugate and Hoechst 33342 were purchased from Life Technologies (Grand Island, NY, USA). γ-³²P labeled ATP (250 μCi) was purchased from PerkinElmer Health Science B. V., The Netherlands. T4 Polynucleotide kinase and 1× Polynucleotide buffer were obtained from New England Biolabs, Frankfurt a. M., Germany. Binding buffer used for the aptamer competition-binding assay was prepared by adding E. coli tRNA (Roche AG, Mannheim, Germany), bovine serum albumin (BSA; Thermo Fischer Scientific) into the Dulbecco's PBS (Gibco, Life Technologies).

All solvents, reagents and building blocks for oligonucleotide synthesis were obtained from Proligo, Hamburg, Germany. The anti-cMet aptamer motif (trCLN3) and its lipid derivatives (trCLN3-L4 & trCLN3.mut-L4) were synthesized according to the phosphoramidite protocol using an ABI 3400 synthesizer (Applied Biosystems). Doxorubicin-carrying DxR-L4 modified with 2′,6′-dimethylazobenzene and C₁₂-lipid tails as well as the fluorescent-labeled (Atto647-, Atto550- and 6FAM) trCLN3-L4 and DxR-L4 motifs were purchased in HPLC purified form from Ella Biotech GmbH, Munich, Germany.

### 1.2 Cell Culture and Confocal Microscopy

The human non-small cell lung cancer (NSCLC) cell line H1838 was obtained from the American Type Culture Collection (ATCC). Cell cultures were tested for mycoplasma contamination by using the PCR-based Venor®GeM Mycoplasma detection kit. Cells were grown in T-75 cm² flasks using Dulbeccos RPMI 1640 (Invitrogen) supplemented with 10% fetal calf serum (FCS) in a humidified atmosphere at 37° C. and 5% CO₂. Cell lines were subcultured twice a week at a ratio of 1:4 depending on the confluence and cell density was determined with a hemocytometer before each experiment. Cells were detached using 1 ml Trypsin-EDTA solution (Sigma-Aldrich) followed by neutralization with 25 ml of RPMI medium and the cells were collected by centrifugation for 5 min at 400 rpm.

In vitro cell imaging of the cell internalization studies were performed using fluorescence microscopy. Prior to each experiment one 70 to 80% confluent flask was trypsinised and suspended with 10 ml of cell medium. 10 μL of the cell solution was pipetted onto a haemocytometer and the cells were counted. Twenty-four hours prior to the internalization experiments approximately 10,000 NSCLC cells were seeded in 96-well glass bottom multiwell cell culture plates (MatTek® Corporation). The plates were then incubated for 24 hours at 37° C. in 5% CO₂-atmosphere. After 24 hours of incubation the cells were first washed with 1×PBS buffer and incubated with various labeled aptameric nanoconstruts (trCLN3-L4, trCLN3.mut-L4, HyApNc-DxR, HyApNc.mut-DxR or free DxR) in 100 μL of RPMI 1640 with 10% FCS medium containing 1 mM MgCl₂ at 37° C. and 4° C. separately for 2 hours. The final concentrations of the labeled micelles were fixed at 10 μM. Afterwards, cells were washed with fresh medium and Dulbeccos 1×PBS followed by 10 min fixation with 200 μL of a 3.7% (w/v) paraformaldehyde solution in Dulbeccos 1×PBS. Fixed cells were washed with fresh medium and Dulbeccos 1×PBS followed by staining with 200 μL of nuclear and plasma membrane staining reagent [60 μL (1 mg/ml) of Alexa Fluor 488-WGA and 20 μL of Hoechst 33342 (1 mM) in 4.0 mL in 1×PBS buffer] and incubated for 10minutes at 37° C. After 10 minutes, the labeling solutions were removed and the stained cells were washed with 1×PBS (2×200 μL) followed by addition of 200 μL of 1×PBS buffer. Finally the 96 well plate was mounted with a multi-well plate holder and the confocal imaging of the fixed cells was performed by using a NikonTi-E Eclipse inverted confocal laser-scanning microscope equipped with a 60× Plan Apo VC Oil-immersion DIC N2 objective, a Nikon C2 plus confocal-laser scan head and a pinhole of 1.2 airy unit (30 μm). The laser scanning Nikon Confocal Workstation with Galvano scanner. and lasers 408. 488. 561 and 637 nm was used, attached to a Nikon Eclipse Ti inverted microscope. Images were captured in 1024×1024 pixels format using NIS-Elements software (Nikon Corporation) and the raw images were processed using ImageJ software. The standardized optical setups of imaging, pin-holes, objective, laser power and photomultiplier gain were kept constant while recording the data for all measurements.

### 1.3 Atomic Force Microscopy (AFM)

All AFM images of the trCLN3-L4 and HyApNc aggregates were taken by using a Nanowizard III AFM (JPK instruments, Berlin) in tapping mode. ACTA probes with silicon tips were used for imaging in dry mode in air. A volume of 3 μL (5 mM) of a solution of magnesium acetate (MgAc₂) in water was deposited on a freshly cleaved mica surface layer and allowed to incubate for 3 minutes and afterwards the surface was rinsed with 2×10 μL of milli-Q water and dried under air pressure. For imaging a volume of 3 μL of the trCLN3-L4/HyApNc solutions in ultra pure water were spotted on the pre-treated mica surface and allowed to incubate for 1 min. After 1 min incubation on the mica surface, the excess sample solution was gently shaken off and the mica surface was blown dry with air pressure and mounted to the AFM microscope for immediate imaging. The raw AFM data were processed using the JPK processing software.

### 1.4 TEM Analysis

The size and structure of the trCLN3-L4 nanoconstructs were analyzed by negative stain electron microscopy. Samples were prepared using negative staining. In brief, carbon coated grids (Quantifoil Micro Tools GmbH, Jena, Germany, 200 mesh) were glow discharged to render the surface hydrophilic prior to applying samples. 10 μL of an aqueous solution of trCLN3-L4 were applied to the grid. Afterwards excess solution was carefully blotted off using filter paper followed by 3 times washing with ddH₂O. In the final step, grids were stained with negative staining reagent by placing them (plastic side down) on a 10 μL drop of freshly prepared 2% (v/v) uranyl formiate aqueous staining solution. TEM micrographs were recorded using a JEOL JEM 2200 FS electron microscope (JEOL, Japan) operated at 200 kV. The size of the micelles measured on the TEM images could typically be observed in a range between 20 and 25 nm.

### 1.5 ESI Mass Spectrometry

Molecular weights of the trCLN3-L4 and DxR-L4 motifs were analyzed by electrospray ionization iquid chromatography mass spectrometry (ESI-LCMS) in negative ion mode using a Bruker Esquire HCT 6,000 ion-trap MS system with an ESI source in line with an Agilent 1100 series HPLC system with a ZORBAX SB-18 analytical column (2.1×50 mm). An elution buffer (10 mM TEA+100 mM HFIP) in combination with linear gradients of acetonitrile from 0% to 80% in 30 minutes was used as mobile phase for analysis. The m/z ratio is calculated by deconvolution of the ionic fragments using Bruker Compass Data Analysis Software.

### 1.6 Serum Stability of trCLN3 with its Lipid Functionalized Derivative

Serum stabilities of trCLN3, its two point mutant non-binding variant trCLN3.mut and their corresponding lipid-functionalized derivatives trCLN3-L4 and trCLN3.mut-L4 were investigated in fetal calf serum (FCS) and human blood serum. For this purpose, the aptamer motifs were labeled at their 5′-end with ³²P to form radiolabeled oligonucleotides. The degradation tests of all the aptamer motifs were performed for 60-72 h at 37°° C. 6 pmol (12 μl of 0.5 μM) of the radio-labeled aptamer (5′-end-labeled with γ-³²P) was incubated in a volume of 300 μl freshly thawed PBS-buffered FCS or human blood serum (270 μl serum+30 μl 10×PBS). For each measurement, 10 μl of the samples were removed, mixed with 90 μl of gel loading buffer (80% formamide+5 mM EDTA+0.01% SDS) and subsequently stored at −20° C. Aliquots of samples were taken after indicated time intervals of 0, 0.3, 1.5, 3, 6, 24, 48, 60 and 72h respectively. The serum stability of the aptamer in FCS or in HBS at different time intervals were analyzed on a denaturing PAGE by loading 10 μl of each sample onto a 10% TAE-Urea gel and running the gels for 90 minutes at 350 V. Gels were wrapped in clingfilm and exposed to a phosphorimager screen in a closed cassette over a period of 12 h and finally the residual intact aptamer bands were analyzed by scanning the screen in a phosphorimage-scanner (FujiFilm FLA 3000). Intensities of the residual intact aptamer bands were calculated applying AIDA image analyzer software program. Serums half-lives of the selected aptamers were determined by using a half-life curve-fitting data analysis program (GraphPad Prism).

### 1.7 Assembly of trCLN3-L4 and HyApNc Nanoconstructs

The fabrication of both the homogeneous nanoconstructs and hybrid micellar nanoconstructs (HyApNc) in aqueous solution, induced by microphase separation, with an outer shell of aptameric DNA and an inner core of the hydrophobic lipids was performed by employing a simple heating and cooling procedure. An aqueous solution of 250 pmol of trCLN3-L4 was added to 250 pmol of DxR-L4 motif dissolved in a volume of 50 μl milli-Q H₂O (10 μM solution). The resulting solution was heated to 90° C. for 10 minutes and subsequently cooled down to a temperature of 10° C. at a rate of 1° C./10 minutes. In case of the aptamers functionalized with fluorescent markers, the solutions were heated up to 70° C. instead of 90° C. and then gradually cooled down to a temperature of 10° C. at a rate of 1° C./10 minute using a thermocycler.

### 1.8 Loading HyApNe Carrier with Doxorubicin

DxR-loaded hybrid-aptameric nanoconstruct (HyApNc-DxR) was prepared by mixing trCLN3-L4 3 with DxR-L4 4 motif in 1:1 ratio with 10-fold excess of DxR in binding buffer (1×PBS+1 mM MgCl2). The solution was incubated at 90° C. for 10 minutes and slowly cooled down to room temperature overnight at a rate of 1° C./10 min in order to intercalate doxorubicin into the DxR-L4 motif. The DxR-loaded HyApNc was transferred to an Amicon® Ultra-0.5 centrifugal filter column with 3K molecular weight cutoff. Free doxorubicin which was not intercalated into DxR-L4 motif was removed by three times consecutive centrifugation at 14.000×g for 10 minutes at room temperature while adding fresh binding buffer at each centrifugation step. After each centrifugation step, a UV/Vis-spectrum of the flow through washing was recorded and a reduction in doxorubicin absorbance further confirmed the successive removal of excess doxorubicin through repeated washing.

### 1.9 Cell Viability Assay

To assess the cytotoxicity of free DxR and HyApNc-DxR in NCI-H1838 lung cancer cells, the H1838 cells (2×10⁴ cells/well) were seeded in a 96 well plate and grown for 24 h. The cells were then washed with 1×PBS (200 μL) and subsequently treated with i) free DxR (as control), ii) HyApNc-DxR or iii) HyApNc.mut-DxR in a dose dependent way with a final DxR concentration ranging from 0.125μM to 50 μM per well. After 2 h of post-treatment, the cells were washed; the RPMI medium was replaced with a fresh RPMI medium, and subsequently either irradiated with UV light for 5 minutes (_=365 nm; 350 mW/cm²), or not irradiated. Afterwards the cells were incubated for another 24 h at 37° C.

For time dependent cytotoxicity assays, H1838 cells were grown at different seeding densities of 10,000, 15,000, 20,000 and 30,000 cells/well in a 96-well plate for 24 h. The cells were then washed with 1×PBS and subsequently incubated with (i) unloaded HyApNc (ii) HyApNc-DxR, (iii) HyApNp_{w/oAz}-DxR with a final DxR concentration of 8 μM in the culture medium. After 2 h of post-treatment, the cells were washed; the RPMI medium was replaced with fresh RPMI medium, and subsequently either irradiated with UV light for 5 minutes (┘=365 nm; 350 mW/cm²), or not irradiated. Then the cells are allowed to culture for another 8, 24 or 48 h respectively.

Then for both experiments, 15 μL of a 3-(4,5-dimethylthiazol-2-yl)-2,5-diphenyltetrazolium bromide (MTT) stock solution (5 mg/mL) was added to each well and the cells were incubated at 37° C. for 6 hours. After 6 h post-tretment with MTT solutions, 100 μL of the SDS-HCL solution was added to each well and mixed thoroughly with a pipette and incubated at 37° C. for an additional 12 hours. Finally the absorbance was measured at λ=570 nm by using a Tecan Infinite® M1000 PRO microplate reader.

### Example 2: Synthesis of trCLN3-L4 and its Two-Point Mutant trCLN3.mut-L4

### 2.1. Synthesis of Lipid-Modified 5′-DMT-2′-deoxyuridine-phosphoramidite

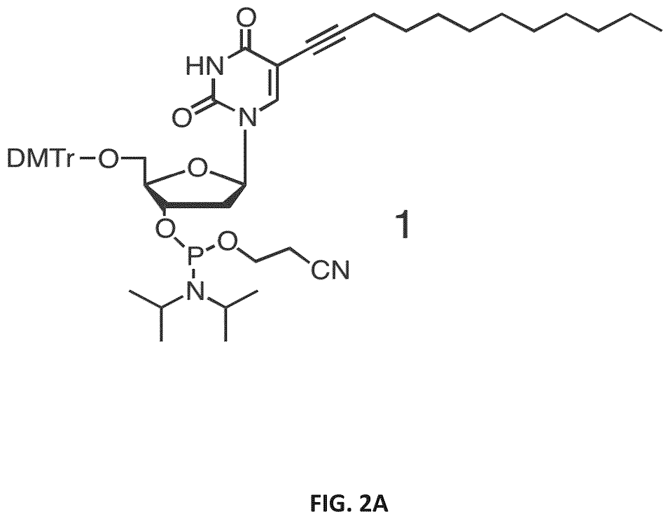

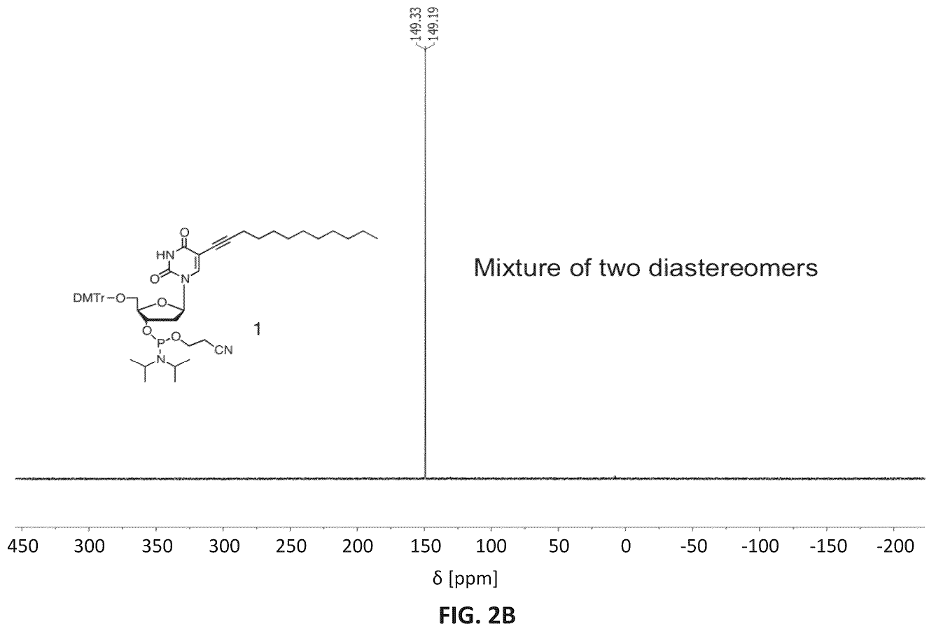

5-(1-Dodecynyl)-modified 5′-DMT-2′-deoxyuridine-phosphoramidite 1 (FIG. 2A) was synthesized from 5-lodo-2′-deoxyuridine as starting material using synthesis protocols reported in a previous study (M. Kwak et al., J. Am. Chem. Soc. 2010, 132, 7834-7835; which reference is incorporated by reference herein in its entirety) and analyzed by ESI mass spectrometry and ³¹P-NMR. Characteristics:

Chemical formula: C₅₁H₆₇N₄O₈P

Molecular weight: 894.47 g/mol

³¹P-NMR: (162 MHZ, CD₂Cl₂) δ[ppm]=149.19 (s), 149.33 (s).

MS: (ESI, positive) m/z (%)=917.5 (16) [M+Na]+, 895.5 (28) [M+H]+, 303.1 (100) [DMT+].

HRMS: (ESI, positive) m/z calculated for C₅₁H₆₇N₄O₈PH [M+H]+895.4769, found: 895.4773

### 2.2. Characterization by ³¹P NMR

³¹P NMR (162 MHZ, CD₂Cl₂) δ[ppm]: 149.19, 149.33. See FIG. 2B.

### 2.3. Lipidated Anti-cMet Aptamer trCLN3-L4 and its Non-Binding Mutant trCLN3.mut-L4

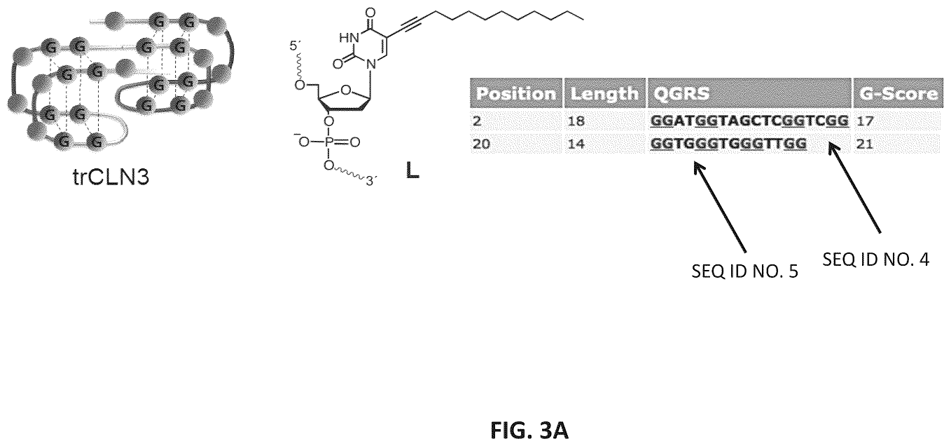

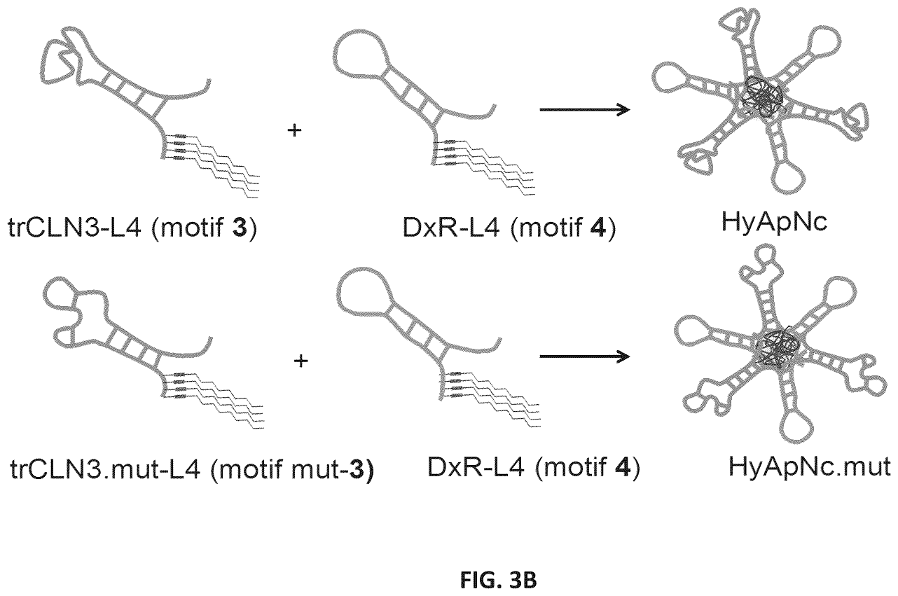

The anti-cMet aptamer trCLN3, a 40 nucleotide DNA oligonucleotide rich in guanine sequence, is known to form two intramolecular G-quadruplex structure within the G-rich segment of the aptamer. See FIG. 3; Table 1. The G-quadruplex structure in trCLN3 is believed to play a role in target recognition and binding to cMet. Filter retention assays with ³²P-labeled variant showed that trCLN3 binds to cMet in nanomolar concentrations. See J. Vinkenborg et al., Angew Chem Int Ed. 2012, 36, 9176-9180; which reference is incorporated herein in its entirety. Binding affinity of trCLN3.mut, a control sequence with a guanine double-point mutation corresponding to G7 and G25 was further verified by filter retention assays. As almost no binding was observed for the two-point mutant control sequence, it was used as non-binding variant in further experiments.

TABLE 1
Sequences

| | SEQ | |
|---|---|---|
| | ID | |
| Name | NO. | Sequence |
| trCLN3 | 1 | 5′-TGGATGGTAGCTCGGTCGGGGT |
| | | GGG TGGGTTGGCAAGTCT-3′ |
| trCLN3.mut | 3 | 5′-TGGATGATAGCTCGGTCGGGGT |
| | | GGA TGGGTTGGCAAGTCT-3′ |
| trCLN3-L4 | Modi- | 5′-LLLLTGGATGGTAGCTCGGTCGGGGT |
| | fied | GGG TGGGTTGGCAAGTCT-3′ |
| | SEQ | |
| | ID | |
| | NO. 1 | |
| trCLN3.mut- | Modi- | 5′-LLLLTGGATGATAGCTCGGTCGGGGT |
| L4 | fied | GGA TGGGTTGGCAAGTCT-3′ |
| | SEQ | |
| | ID | |
| | NO. 3 | |

Four C₁₂-lipid chains (L) were coupled to trCLN3 in a single process using a standard phosphoramidite solid-phase DNA synthesis protocol. trCLN3-L4 and its non-binding mutant trCLN3.mut-L4 with the 40 nucleotide sequence (see FIG. 3A) both were synthesized in scale of 200 nmol scale using an ABI 3400 DNA synthesizer. The lipid-modified uridine-phosphoramidite 1 (0.221 g) was dissolved in DNA-grade dichloromethane (2.7 mL) under argon atmosphere to give a 0.1 M solution. Synthesis of trCLN3-L4 and trCLN3.mut-L4 was performed identically, except for the building-up of the oligodinucleotide (ODN) sequences. After the last detritylation step the lipidated-uridine phosphoramidite 1 was coupled to the detritylated 5′-end of the oligonucleotide chain, using an optimized coupling procedure. Subsequently deprotection of phosphate groups and protected amino nucleobases as well as cleavage of the product from the solid support was carried out by incubation in a 50:50 (v/v) mixture of 30% ammonia solution (400 μl) and methyl amine (400 μl) for 2 h at 55° C. The solid support was then removed by filtering and was washed with an ethanol/water (50:50, v/v) mixture. The filtrate was concentrated under reduced pressure and dried.

### 2.4. Reversed-Phase HPLC Purification

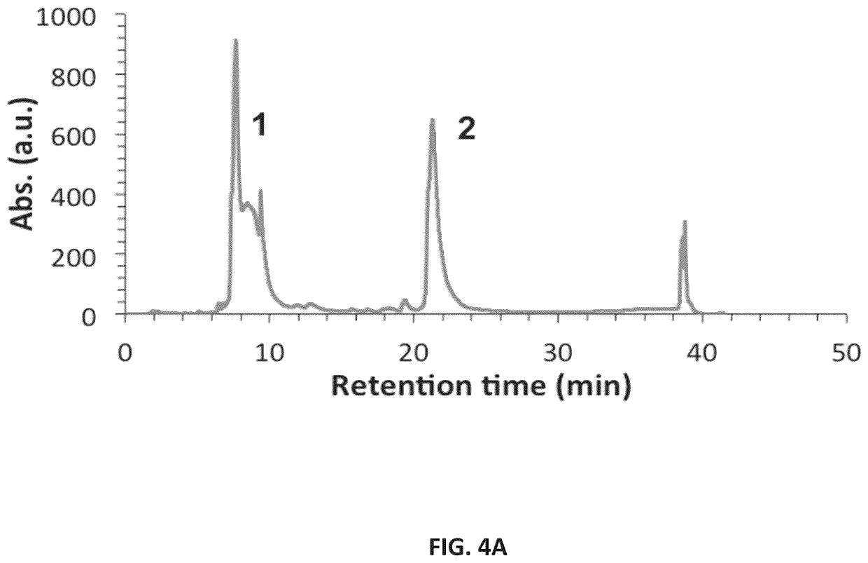

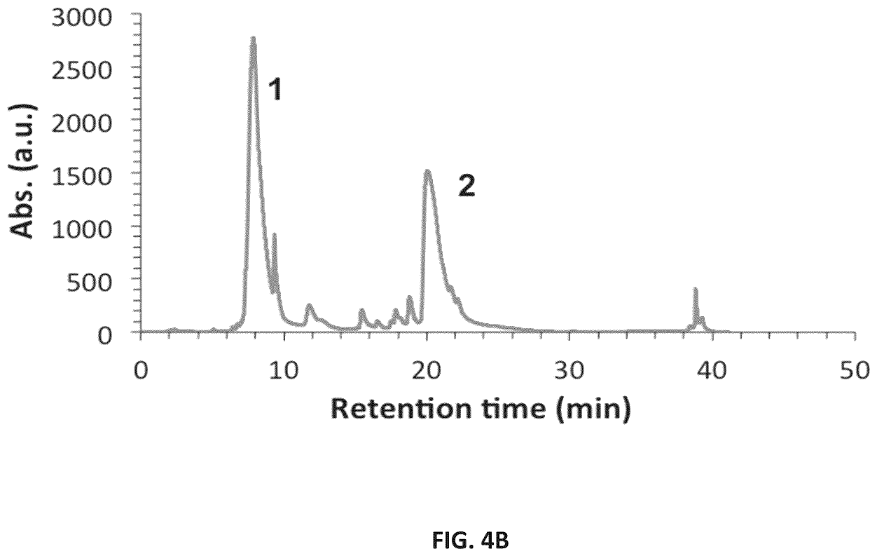

Following deprotection and separation from the solid-support, the lipid-functionalized aptamers trCLN3-L4 & trCLN3.mut-L4 were purified by using reversed-phase high performance liquid chromatography (HPLC) on an Eclipse XBD C18 column using 0.1 M TEAAc (A) and acetonitrile (B) with a gradient of A/B=98/2->35/65 in 30 minutes. The coupling yield of the labeling reaction was determined to be 31% trCLN3-L4 and 29% trCLN3.mut-L4 respectively by integration of the peaks in the HPLC chromatogram. See FIGS. 4A, FIG. 4B, respectively. The purified lipid-modified oligonucleotide fraction was concentrated using a freeze-dryer. Oligonucleotide concentrations were determined by UV absorbance using extinction coefficients at ┘=260 nm. The identity of the oligonucleotides was confirmed by ESI-mass spectrometry as described below.

### 2.5. ESI Mass Spectrometry

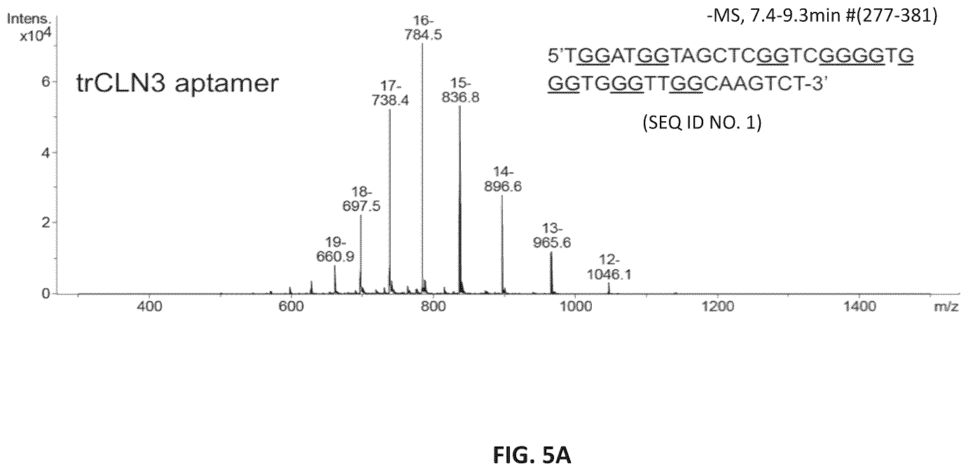

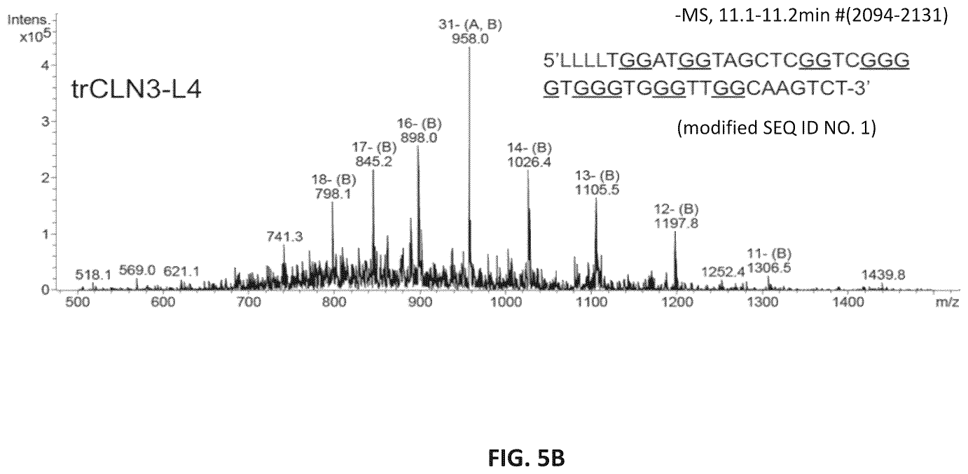

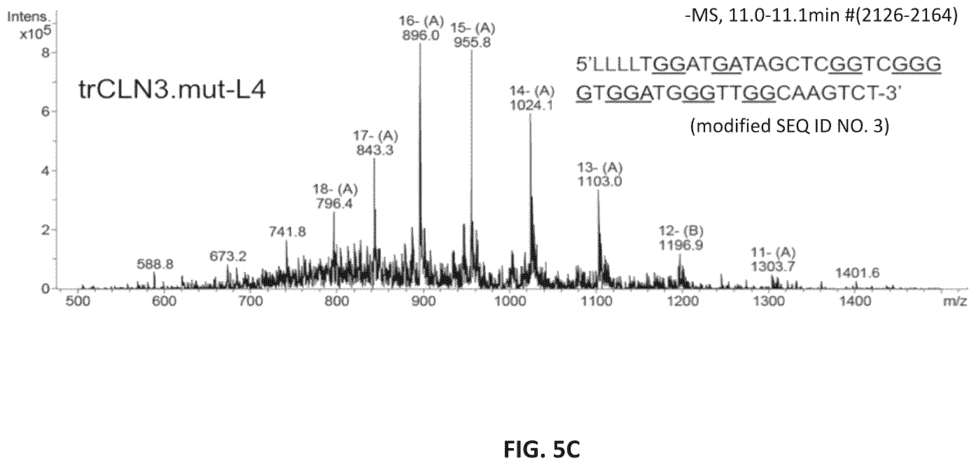

The molecular masses of anti cMet aptamer trCLN3 and its lipid-functionalized derivatives were further analyzed by ESI-LCMS in negative ion mode using a Bruker Esquire 6,000 ion-trap MS system with an electrospray ionization source coupled to an Agilent 1100 series HPLC system modified with a ZORBAX SB-18 analytical column (2.1×50 mm). The ESI mass spectra of the purified trCLN3 aptamer and its lipid-functionalized conjugates are shown in FIGS. 5A-C. An elution buffer (10 mM triethanolamine (TEA)+100 mM hexafluoroisopropanol (HFIP)) in combination with linear gradients of acetonitrile from 0% to 80% in 30 minutes was used as mobile phase for analysis. The m/z ratio is calculated by deconvolution of the ionic fragments.

### Example 3: Critical Micelle Concentrations of trCLN3 Aggregates

### 3.1. Critical Micelle Concentrations via FRET Studies

The critical micelle concentration (CMC) value of the trCLN3-L4 aggregates was determined by intermolecular Förster resonance energy transfer (FRET) experiments using a FRET pair of 6-Fam and Atto647N both attached to the 5′-end of the trCLN3-L4 motif 3. The FRET labels were attached at the 5′-end in immediate proximity to the lipid-modifications to ensure that intermolecular FRET effects report the formation of micellar nanoconstructs at a concentration above the critical micelle concentration.

In the FRET experiment, a series of nanoconstructs was self-assembled by mixing 6-Fam- and Atto647N-labeled motif 3 in 1:1 ratios in a concentration range between 0.035-15 μM (Table 2). The solutions were incubated at 70° C. for 10 minutes in the dark and slowly cooled down to room temperature overnight at a rate of 1° C. per 10 minutes. The mixtures were transferred into a 384-well plate and the FRET effect was monitored at room temperature by using an excitation wave length of λₑₓ=480 nm and an emission wavelength of λₑₘ=669 nm using an EnSpire® Multimode Plate Reader (PerkinElmer).

TABLE 2
Concentrations of 6-Fam- and Atto647N-labeled motifs 3
mixed in 1:1 ratios to form mixed micellar nanoconstructs

| | | | | Ratio | | | |
|---|---|---|---|---|---|---|---|
| Exp. | 6fam-3 | atto647-3 | Volume | 6fam: | | | I₆₆₉/ |
| No. | [μM] | [μM] | (μL) | atto647 | I₆₆₉ | I₅₂₀ | I₅₂₀ |
| 01 | 10.0 | 10.0 | 20 | 1:1 | 17481 | 4152 | 4.21 |
| 02 | 5.0 | 5.0 | 20 | 1:1 | 10876 | 2254 | 4.82 |
| 03 | 2.5 | 2.5 | 20 | 1:1 | 5176 | 1062 | 4.87 |
| 04 | 1.0 | 1.0 | 20 | 1:1 | 1526 | 501 | 3.04 |
| 05 | 0.5 | 0.5 | 20 | 1:1 | 585 | 434 | 1.35 |
| 06 | 0.25 | 0.25 | 20 | 1:1 | 71 | 139 | 0.51 |
| 07 | 0.125 | 0.125 | 20 | 1:1 | 97 | 114 | 0.85 |
| 08 | 0.07 | 0.07 | 20 | 1:1 | 16 | 78 | 0.21 |
| 09 | 0.035 | 0.035 | 20 | 1:1 | 6 | 126 | 0.05 |

The intensity signals were collected for both FRET channels at λₑₘ=669 nm for the acceptor channel and that of donor channel at λₑₘ=520 nm. The concentration dependent intensity ratios (I₆₆₉/I₅₂₀) were plotted as a logarithmic function depending on the trCLN3-L4 concentration. The CMC value was determined from the intersection of the lower horizontal asymptote of the sigmoidal curve with the tangent at the inflection point corresponding to the minimum trCLN3-L4 concentration required for formation of stable micelles in aqueous medium. The CMCs of trCLN3-L4 aggregate was determined to be 300 nM (˜0.005 mg/ml).

### 3.2. Critical Micelle Concentrations from Pyrene Fluorescence

Critical micelle concentration (CMC) value of the trCLN3-L4 motif was further confirmed by internalizing pyrene into the hydrophobic-lipid core of the micellar aggregate followed by measuring the fluorescence of pyrene-loaded trCLN3-L4 nanoconstructs at different concentrations. For this experiment a fixed amount of pyrene in acetone was transferred to an empty tube and acetone was allowed to evaporate in the dark at 45° C. for 30 min using an Eppendorf concentrator. trCLN3-L4 solutions in the concentration range between 0.0005-0.5 mg/ml were then added to yield a final pyrene concentration fixed at 100 μM for all reactions (Table 3). The solutions were incubated at 90° C. for 10 minutes in the dark and slowly cooled down to room temperature overnight at a rate of 1° C./10 min in order to internalize pyrene into the hydrophobic lipid core. The pyrene-loaded trCLN3-L4 nanoconstructs were transferred into a 384-multi well plate and the fluorescence emission spectrum of each well was recorded at room temperature by using an excitation wave length of 339 nm in an EnSpire® Multimode Plate Reader (PerkinElmer).

TABLE 3
Concentrations for trCLN3-L4 3 micelles and pyrene in a
fixed reaction volume of 50 μl used for CMC determination
of trCLN3-L4 aggregated nanoconstructs

| | trCLN3- | trCLN3- | | Vol- | | | |
|---|---|---|---|---|---|---|---|
| Exp. | L4 3 | L4 3 | | ume | | | I₄₇₅/ |
| No. | [mg/mL] | [μM] | Pyrene[μM] | [μL] | I₄₇₅ | I₃₇₃ | I₃₇₃ |
| 01 | 0.5 | 35 | 100 | 50 | 174827 | 22321 | 7.83 |
| 02 | 0.25 | 17.4 | 100 | 50 | 130337 | 18886 | 6.90 |
| 03 | 0.1 | 7.0 | 100 | 50 | 90719 | 17675 | 5.13 |
| 04 | 0.05 | 3.5 | 100 | 50 | 60458 | 12887 | 4.69 |
| 05 | 0.025 | 1.75 | 100 | 50 | 47267 | 18925 | 2.49 |
| 06 | 0.01 | 0.7 | 100 | 50 | 41638 | 26517 | 1.57 |
| 07 | 0.005 | 0.35 | 100 | 50 | 40004 | 20218 | 1.98 |
| 08 | 0.0025 | 0.175 | 100 | 50 | 14658 | 20188 | 0.72 |
| 09 | 0.001 | 0.07 | 100 | 50 | 4435 | 19581 | 0.23 |
| 10 | 0.0005 | 0.035 | 100 | 50 | 2751 | 13370 | 0.20 |

In close proximity, two pyrene molecules form an excimer that emits fluorescence at a longer wavelength compared to the monomer emission. The formed excimer is a dimeric complex where one molecule exists in an excited state and the other molecule in a ground state. Monomer emission of pyrene occurs within a range of 360-400 nm whereas the excimer emission is obtained within the wavelength limit of 465-500 nm. The critical micelle concentration was determined by the distinguishable pyrene excimer fluorescence of the corresponding DNA concentration. See G. Uddin G et al., Am. J. Biochem. Mol. Biol. 2013, 3, 175-181; which reference is incorporated herein in its entirety.

### Example 4: Assembly of Anti-cMet Nanoconstructs that Target NCI-H1838 Cells

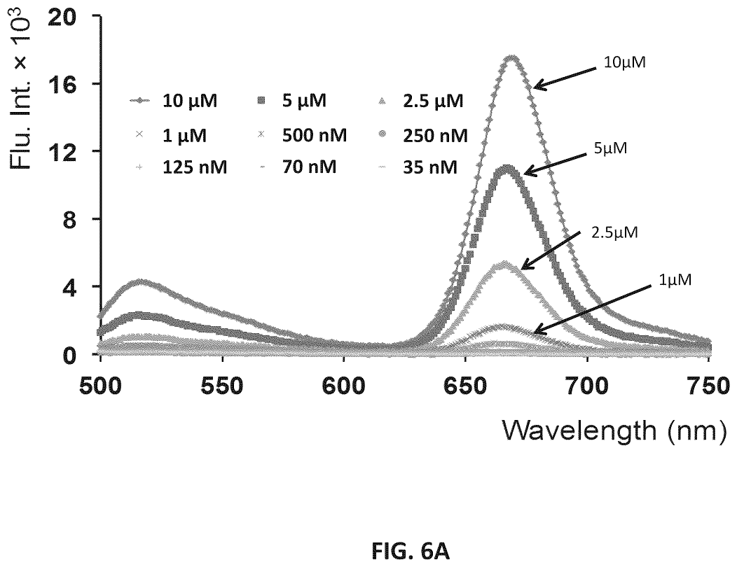

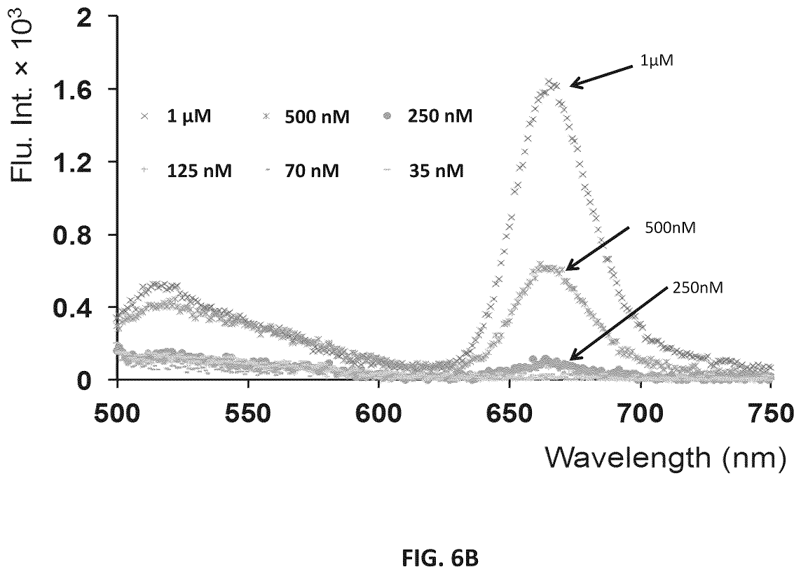

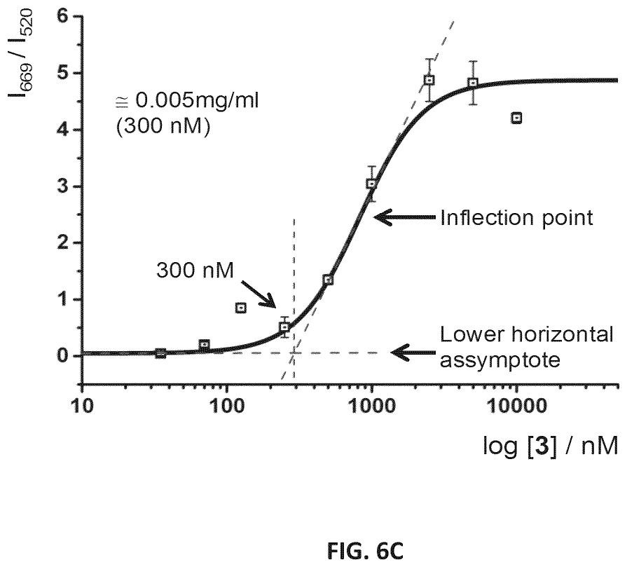

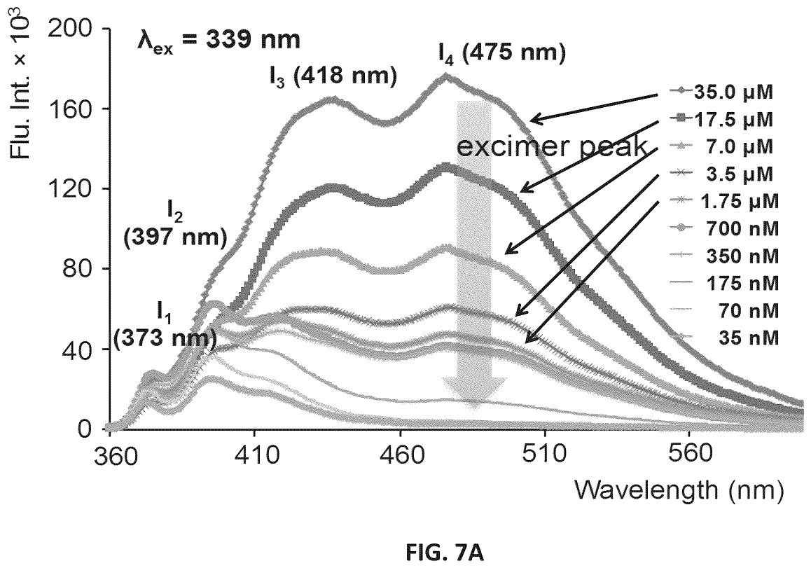

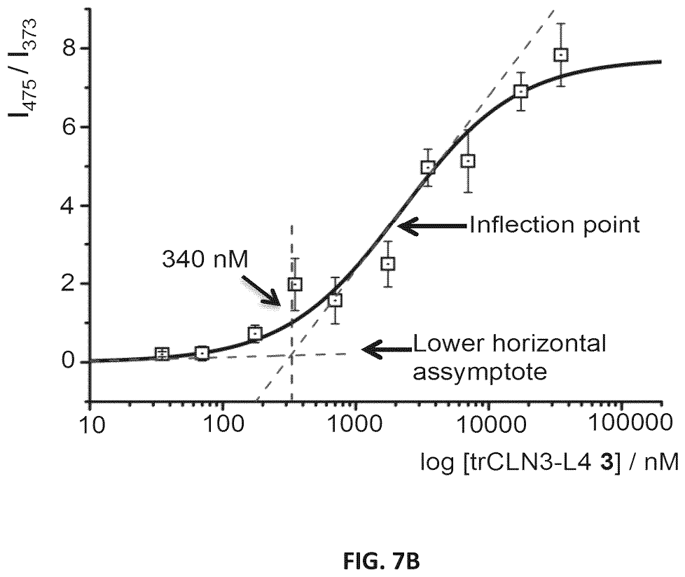

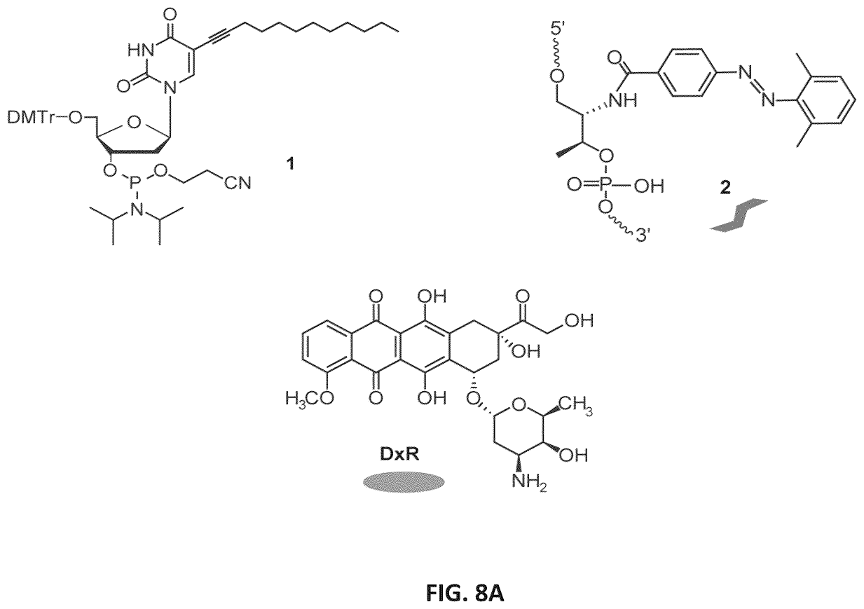

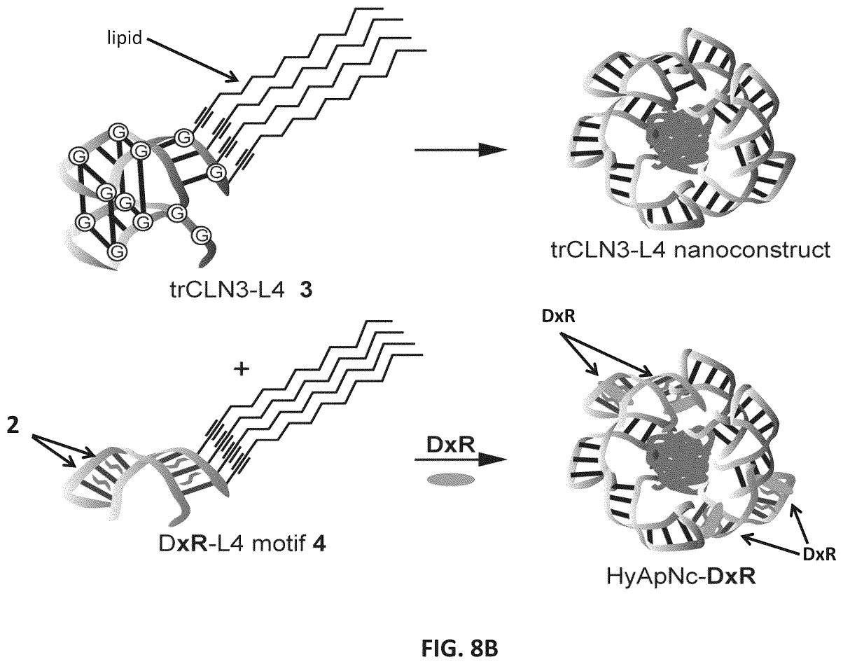

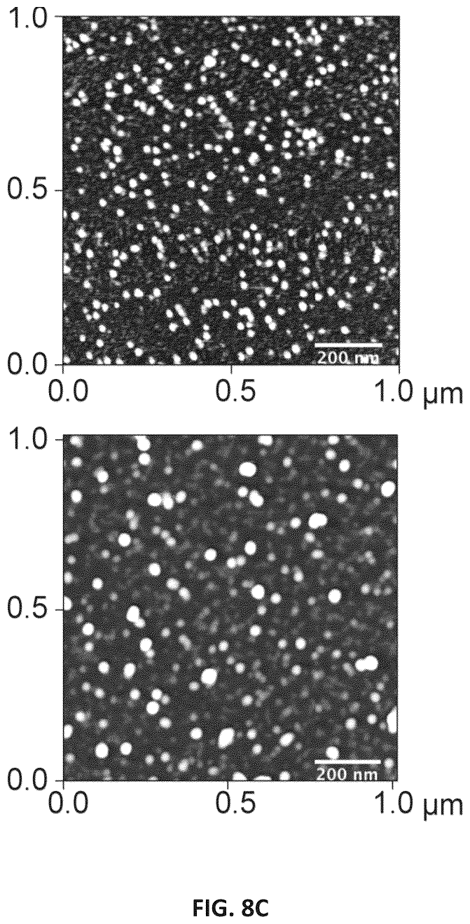

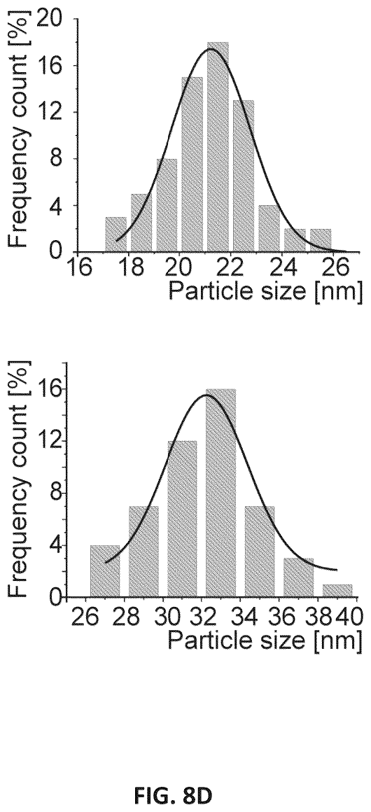

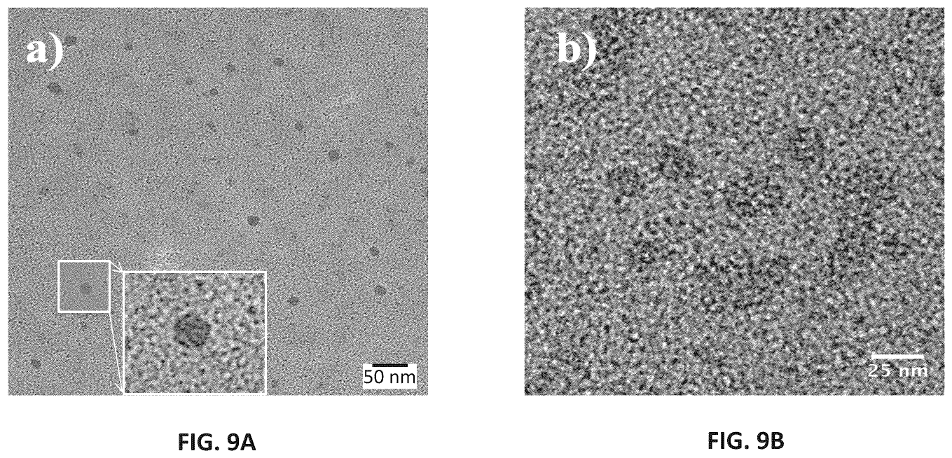

To exemplify the invention, we used the 40-nucleotide anti-cMet DNA aptamer trCLN3 that binds to HGFR (cMet) with a dissociation constant (K_{d}) of 38 nM. cMet is overexpressed on the surface of several types of cancer cells, including the NCI-H1838 lung cancer cell-line used here. In a first step. we synthesized the lipid-modified phosphoramidite 1 with a C₁₂-lipid chain incorporated at the 5-position of the uridine base (FIGS. 2A-B). Four of these modified bases were attached to the 5′-end of the trCLN3 aptamer (see FIGS. 3A-B), thereby introducing four lipid tails into each aptamer. The resulting lipid-functionalized aptamer trCLN3-L4 (3) was purified by reversed-phase HPLC (see FIGS. 4A-B) and confirmed by LCMS mass spectrometry (see FIGS. 5A-C). Polyacrylamide gel electrophoresis (PAGE) of lipidated and non-lipidated trCLN3 aptamers showed significant differences in the migration behavior. consistent with L4-modification (data not shown). Moreover, the L4-modified aptamers showed a strong tendency to self-aggregate in aqueous solution by forming spherical nanoconstructs above a critical micelle concentration (CMC) at room temperature. We evaluated the CMC of the trCLN3-L4nanoconstructs using Forster resonance energy transfer (FRET; Example 3; FIGS. 6A-C; Table 2) and fluorescence studies with pyrene-loaded trCLN3-L4 nanoconstructs (FIGS. 7A-B; Table 3). Both methods yielded CMC values in the range of 300-350 nM concentrations. The size and morphology of the nanoconstructs were further studied by atomic force microscopy (AFM; FIG. 8C, upper panel) and electron microscopy (TEM; FIGS. 9A-B). To obtain a statistical evaluation of the size-distribution of nanoconstructs, the diameters of at least 50 nanoconstructs for each AFM image were compiled in histograms and fitted by Gaussian distributions (FIG. 8D). The trCLN3-L4 3 nanoconstructs have an average diameter of 21.2±1.5 nm (FIG. 8C, upper panel), consistent with the size of 25 nm measured by TEM.

### Example 5: Effect of Lipid-Modifications on cMet Binding and Serum Nuclease Stability

### 5.1. Competitive Filter-Binding Assay

To test the effect of lipid-modification on trCLN3 binding properties, we determined IC₅₀ values for each trCLN3 derivative by a competitive filter retention assay in which varying concentrations of unlabeled 5′-(1-dodecynyl)-functionalized trCLN3 aptamers competed with constant amounts of γ-³²P-labeled trCLN3 in binding to cMet. Two control experiments were also performed using unlabeled trCLN3 and its two point mutant variant trCLN3.mut as competitors.

First, the trCLN3 motif was 5′-end-labeled with γ-³²P ATP using T4 polynucleotide kinase. An aliquot of 20 μL solution containing 50 pmol trCLN3. 6.7 pmol γ-³²P ATP and 20 U T4 polynucleotide kinase in 1× polynucleotide kinase buffer (New England Biolabs) was incubated at 37° C. for 45 min. followed by removal of unreacted γ-³²P ATP using an Illustra G-25 microspin column (GE Healthcare. München, Germany). The purity of the radiolabeled aptamer was confirmed using a 10% PAGE-gel.

To determine the affinity. ˜25 fmol of radiolabeled aptamer was incubated with a cMet concentration of ˜50 nM together with varying concentrations (1 μM-25 pM) of unlabeled competitor for 30 min at 37° C. in 25 μL of buffer containing 0.1 mg/ml E. coli tRNA (Roche, Mannheim, Germany). 0.25 mg/ml BSA. 2 mM MgCl₂ in 1×PBS, pH 7.4. The aptamer-protein complexes were captured on a Protran nitrocellulose membrane (GE Healthcare) that was pre-incubated in 0.4 M KOH for 10 minutes, followed by washing with 1×PBS containing 2 mM MgCl₂, pH 7.4. After addition of the aptamer-protein solution, the filter was washed 4 times with 1×PBS containing 2 mM MgCl₂ using vacuum filtration. Residual radioactivity due to cMet bound labeled aptamers was quantified using Fujifilm Fla-3000 PhosphorImager and AIDA software. The curves were fitted with GraphPadPrism 3.02 plotting non-linear regression curve and the IC₅₀ values have been calculated assuming a competition for single binding site.

### 5.2. Results

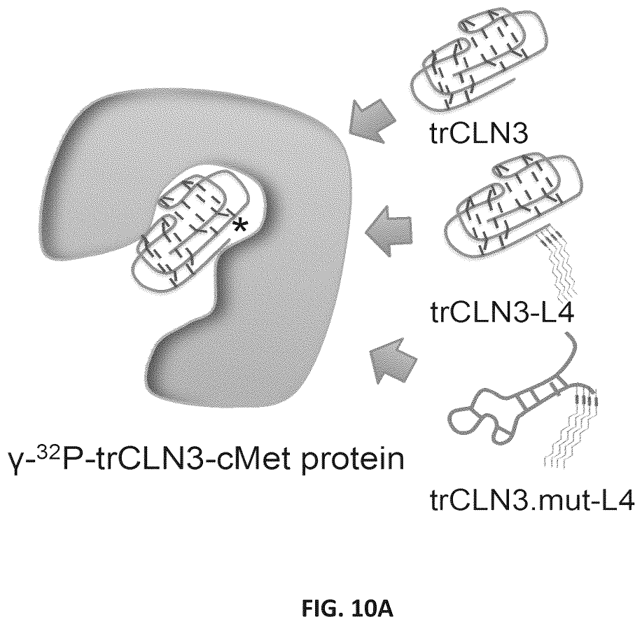

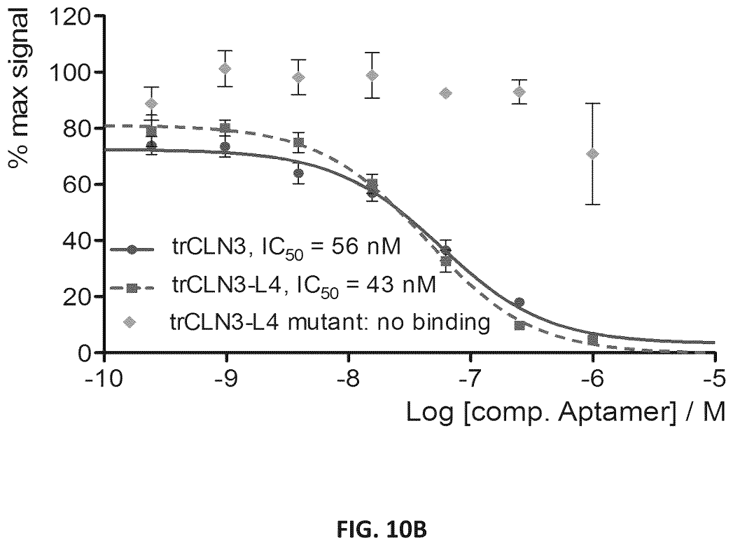

To test the influence of lipid tails on aptamer binding, a competitive filter-binding assay was carried out using the methodology above. Varying concentrations of unlabeled 5′-lipid functionalized aptamer 3 and its two-point mutant variant trCLN3.mut-L4 (see Example 2.3) competed with a constant amount of ³²P-radio-labeled native trCLN3 aptamer in binding to cMet. Strong cMet binding was observed for trCLN3-L4 with an IC₅₀ value of 43 nM, compared to 56 nM obtained for the non-lipidated native aptamer trCLN3 (FIG. 10B). This result demonstrates that aptameric nanoconstructs retained their binding affinity to cMet as compared to the non-modified aptamer trCLN3. In contrast, the lipidated mutant aptamer trCLN3.mut-L4 containing two point mutations could not compete with the ³²P-trCLN3 for binding to cMet within the tested concentration range, indicating that the displacement of the non-lipidated ³²P-trCLN3 from its bound cMet-target by its lipidated counterpart trCLN3-L4 is specific.

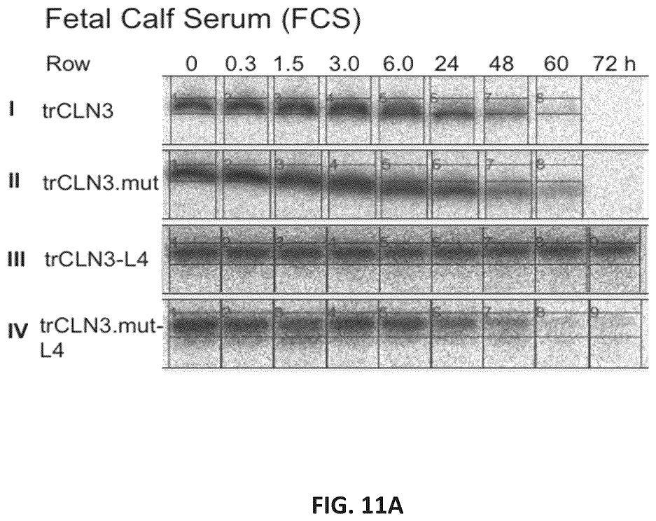

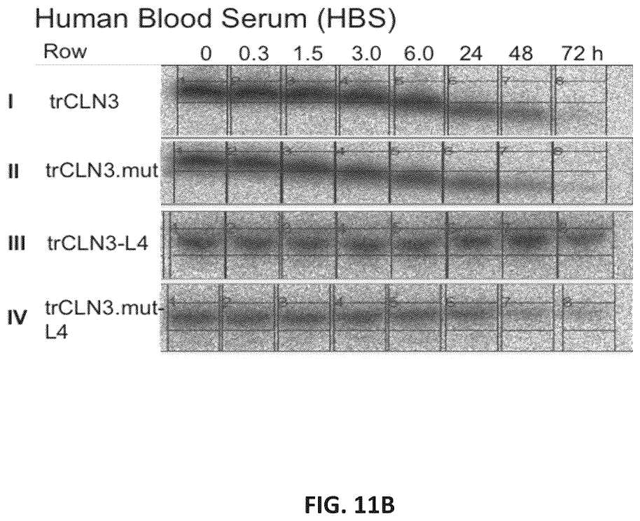

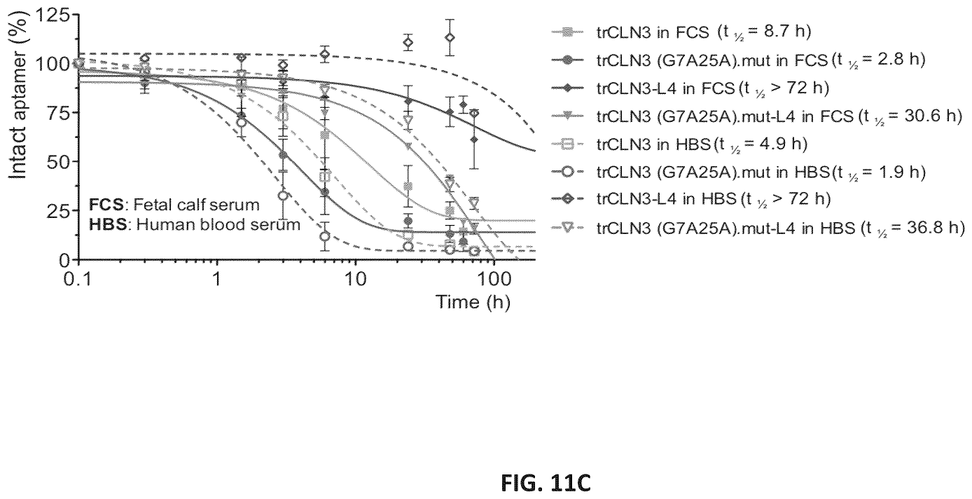

Since an adequate serum half-life is a prerequisite for the successful in vivo application of these aptamers, the serum stabilities of aptamer trCLN3, its double point mutant non-binding variant trCLN3.mut, and their corresponding lipid-functionalized derivatives (trCLN3-L4 & trCLN3.mut-L4, respectively) were analyzed in 10% PBS-buffered fetal calf serum (FCS, FIG. 11A) and in freshly prepared human blood serum (HBS, FIG. 11B) at 37° C. from 0 to 72 h. A comparison of degradation profiles between FCS and HBS revealed similar patterns of aptamer degradation for both serum samples (FIGS. 11A-C). The non-lipidated variants of the aptamer samples degraded 1.5 fold faster in HBS compared to FCS. Under similar conditions the serum half-life (t_{1/2}) of trCLN3 was 8.7 h (10% PBS-buffered FCS) and 4.9 h (10% PBS-buffered HBS), respectively compared to its lipid-functionalized derivative trCLN3-L4 showing no significant degradation even up to 72 h of incubation in both sera. To examine the possibility that the differences in serum stability are due to the G-quadruplex present in both trCLN3-L4 and trCLN3, we also compared serum stabilities of trCLN3.mut-L4 and trCLN3.mut, both not capable of forming a G-quadruplex. The t_{1/2} values of trCLN3.mut-L4 in FCS (≅30.6 h) and in HBS (≅36.8 h), respectively was approximately 10- and 19-fold higher than that of the non-lipidated variant trCLN3.mut (t_{1/2}=2.8 h in FCS; 1.9 h in HBS). See FIG. 11C. These observations indicate that the serum stability of the mutant aptamer is lower than that of trCLN3 native aptamer, and lipidation further protects the aptamer against enzymatic degradation thereby increasing the serum stability several fold.

### Example 6: Design of a Photoswitchable DxR-Binding-Motif

### 6.1. Synthesis of DMT-Protected 2′,6′-dimethylazobenzene phosphoramidite

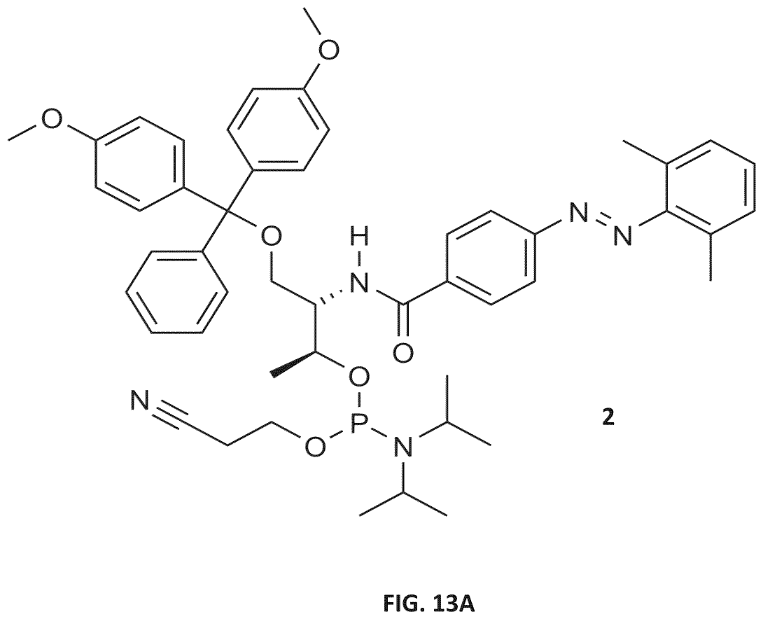

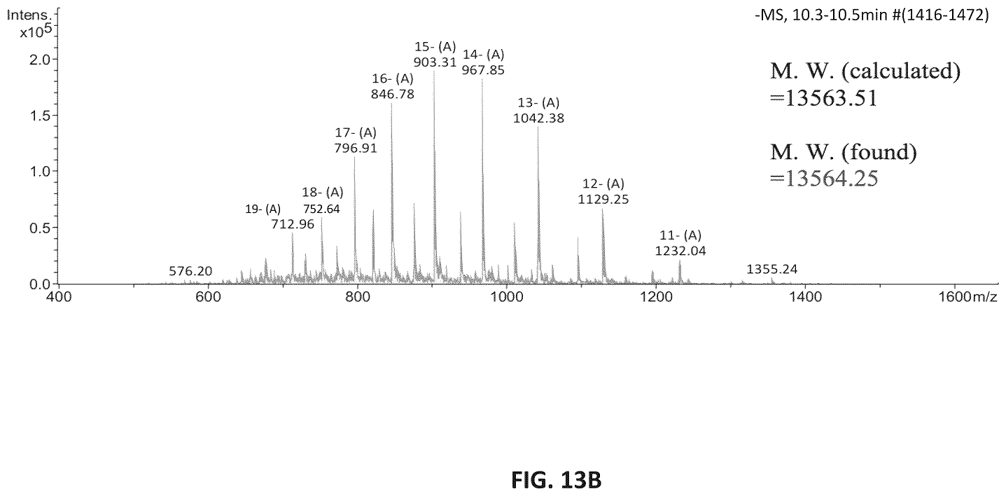

DMT-protected phosphoramidite carrying a 2′,6′-dimethylazobenzene moiety on a D-threoninol backbone (FIG. 13A, 2) was synthesized as reported elsewhere. See C. H. Stuart et al., Bioconjugate Chem. 2014, 25, 406-413; which reference is incorporated by reference herein in its entirety.

### Characteristics:

Chemical formula: C₄₉H₅₈N₅O₆P

Molecular weight: 843.99 g/mol

R_{f}-value: 0.60-0.65 (4 spots, eluent: ethyl acetate and cyclohexane with a volume ratio of 1:1 with 3% triethylamine).

13P-NMR: (162 MHZ, CDCl₃) δ [ppm]=148.72, 149.16.

MS: (ESI, positive) m/z(%)=866.4 (100) [M+Na]⁺, 303.1 (92) [DMT]⁺.

HRMS: (ESI, positive) m/z calculated for C₄₉H₅₈N₅O₆PNa: 866.4017 [M+Na]⁺, found: 866.4011 [M+Na]⁺.

### 6.2. Synthesis of doxorubicin-Carrying DxR-L4 (Motif 4)

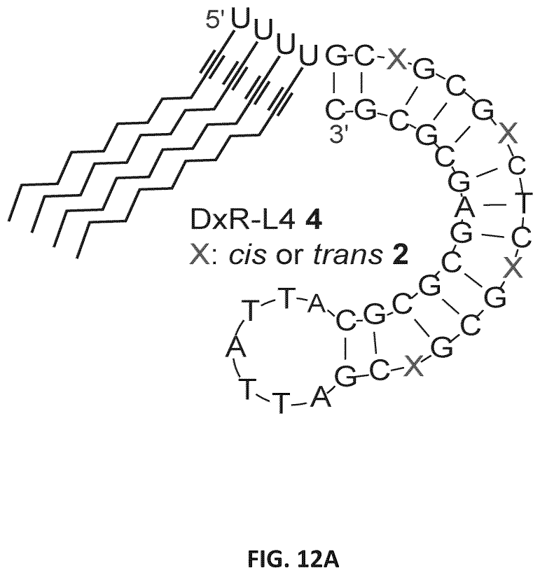

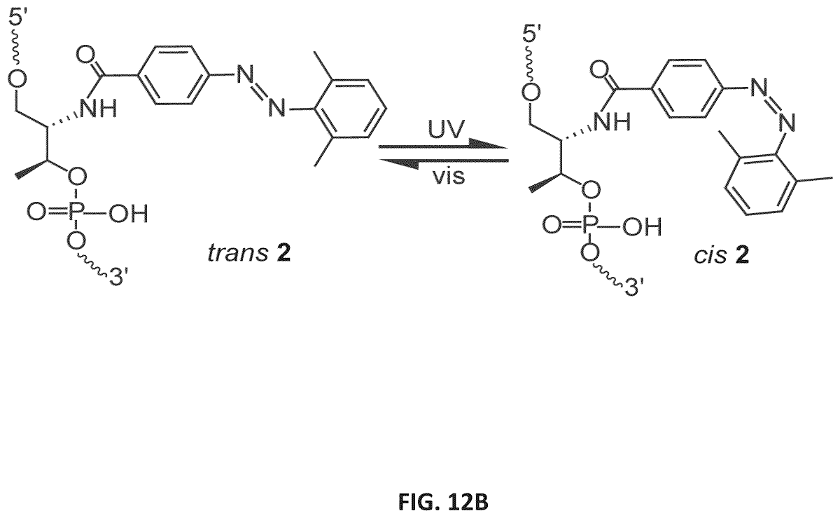

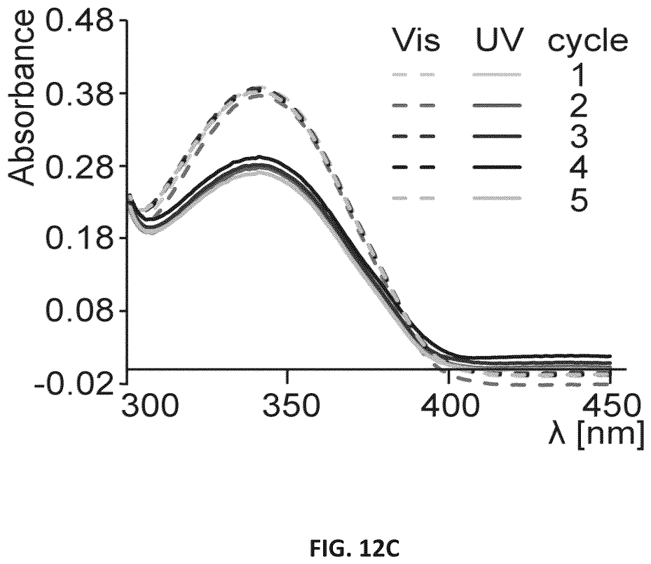

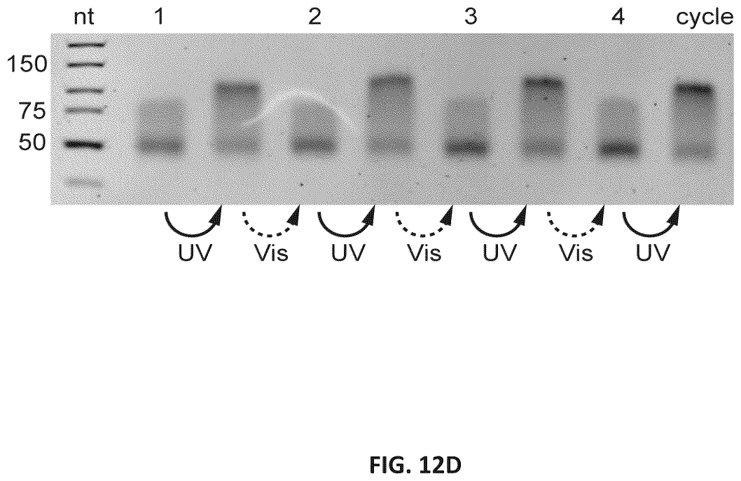

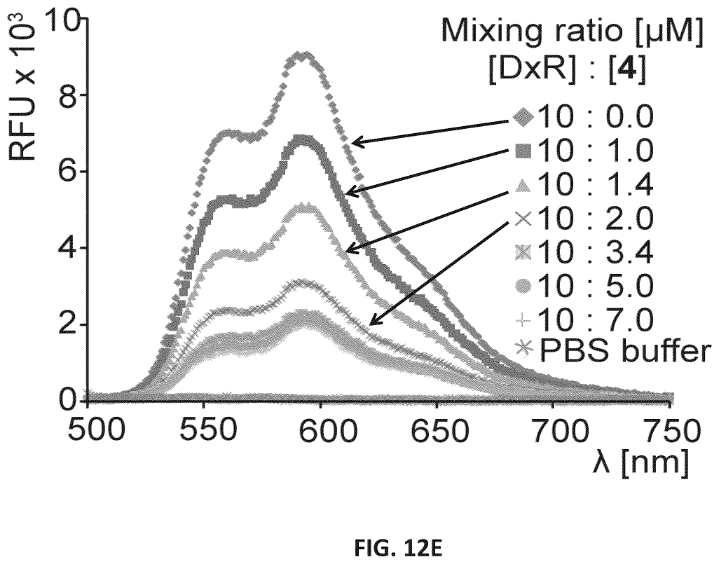

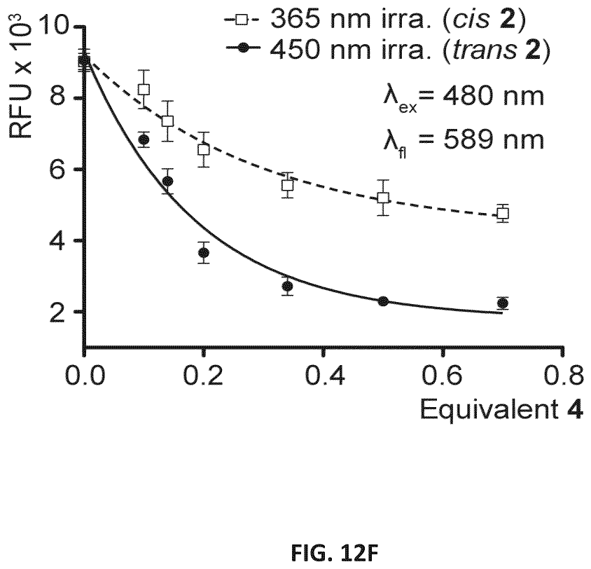

DMAB-phosphramidite and lipid-phosphoramidite were introduced as a photo-trigger and lipid-tails to the doxorubicin carrying DxR-L4 motif by solid phase DNA-synthesis The motif consists of a 37-nucleotide DNA sequence with 4 DMAB moieties introduced into the sequence and four lipid-tails attached to the 5′-end. The resulting purified doxorubicin-carrying DxR-L4 (4) motif (FIG. 12A) was analyzed by ESI-LCMS mass spectrometry.

### 6.3. ESI Mass Spectrometry

The molecular mass of lipid-functionalized DxR-L4 motif 4 was analyzed by ESI-LCMS in negative ion mode (Bruker Esquire 6,000 ion-trap MS system with an electrospray ionization source coupled to an Agilent 1100 series.) The ESI mass spectrum of the purified lipid-functionalized DxR-L4 motif 4 is shown in FIG. 13B. Deconvolution of the ionic fragments leads to a measured total mass of MWmeas=13564.25 corresponding to the target oligonucleotide with the calculated mass of MWcalc=13563.51.

### 6.4. DxR Intercalation to Motif 4 and Purification

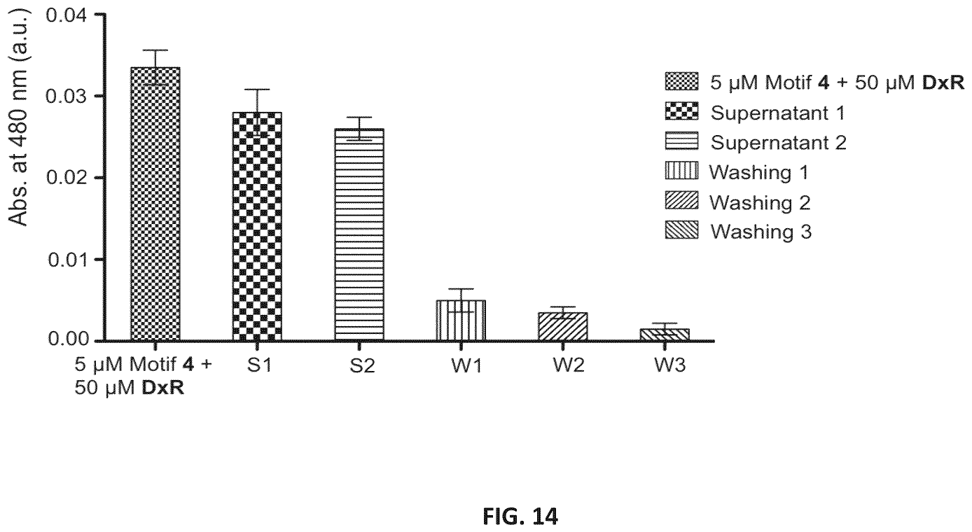

A fixed amount of motif 4 (5 μM) was added to 10-fold excess of DxR in buffer (1×PBS+1 mM MgCl₂) and incubated for 12 h at room temperature. The motif 4-DxR complex was transferred to an Amicon® Ultra-0.5 centrifugal filter column with 3K molecular weight cutoff and excess of free doxorubicin was removed by three rounds of consecutive centrifugation at 14,000 g for 10 minutes at room temperature while adding fresh buffer at each centrifugation step. After each centrifugation step, a UV-Visible (UV/Vis) spectrum of the supernatant and flow through washing was recorded and a reduction in doxorubicin absorbance further confirmed the successive removal of excess doxorubicin through repeated washing. See FIG. 14.

### 6.5. Quantification of the DxR Release from Loaded Motif 4 by HPLC Assay

The release of DxR bound to motif 4 was analyzed by Ion-Exchange chromatography on a TSKgel DEAE-NPR Guard 2.5 μm 4.6×5 mm column (Millipore Sigma). A mobile phase of 1×PBS buffer+5% acetonitrile (ACN; mobile phase A) and 1×PBS buffer+1M NaCl+5% ACN (mobile phase B) were used with a gradient of A/B=100/0->0/100 over 20 minutes. A fully encapsulated motif 4-DxR complex was incubated at 37° C. in 1×PBS buffer. For each measurement an aliquot of 20 μl sample solution was removed after the indicated time interval and irradiated with 365 nm light for 5 minutes. Samples that were not irradiated were used as controls. Following the UV exposure, the samples were extracted twice with phenol/CHCl₃ and twice with CHCl₃, which removed the excess DxR released by photoirradiation. It was already reported that the phenol/CHCl₃ (1:1) washing removes unbound excess Doxorubicin after intercalation into DNA duplexes without removing the intercalated Doxorubicin. See C. H. Stuart et al., Bioconjugate Chem. 2014, 25, 406-413; which reference is incorporated by reference herein in its entirety. Afterwards, 10 μl of each sample was injected and the remaining DxR bound to motif 4 was quantified by recording the fluorescence at 590 nm (λₑₓ=490 nm) using a flow-through fluorescence detector attached to the HPLC.

### 6.6. Results

We synthesized the thermodynamically stable lipid-modified DNA motif 4 consisting of a preferred DxR-binding 37 nucleotide alternating GC sequence combined with four 2′,6′-dimethylazobenzene (DMAB) moieties and 4-lipid tails attached to the 5′-end (FIG. 12A-B). Motif 4 was designed to bind and release DxR reversibly by irradiating with UV- or visible light and the integrity of the DxR-L4 motif 4 was confirmed by LC-MS (FIG. 13B). Reversible photoswitching of the four DMAB-groups contained in motif 4 was investigated by UV/vis-spectroscopy. The switching process is fully reversible and can be repeated for at least 5 irradiation cycles. See FIG. 12C. which shows five cycles yield identical absorbance. This result is further supported by gel electrophoresis of the DMAB-modified GC-rich hairpin structure that showed a change in electrophoretic shift upon repeated irradiation with UV-and visible light for 5 minutes each (FIG. 12D), consistent with significant structural changes between the hairpin and dehybridized motif.

The goal of intercalating and efficiently delivering multiple DxR molecules per motif 4 was investigated by binding studies between motif 4 and DxR. A fixed concentration (10 μM) of DxR was incubated with an increasing molar ratio of motif 4 (1-7 μM) and fluorescence quenching due to intercalation of DxR was used to examine the binding efficiency. Gradual decrease of the fluorescence intensity of DxR was observed upon binding to increasing amounts of motif 4 (FIG. 12E). We further tested the difference in binding affinity of motif 4 for cis- and trans-conformation of the DMAB groups. To do so, motif 4 was separately irradiated with visible light (┘=450 nm) and UV light (_=365 nm) for 5 minutes each and mixed with a fixed concentration of DxR (10 μM) while the concentration of motif 4 was varied from 0.1-0.7 equivalents to that of the DxR concentration. The fluorescence curve of motif 4 with DMAB in trans-conformation (λ=450 nm) showed a higher reduction in fluorescence intensity with an increasing molar equivalent of added motif 4 as compared to 4 in which the DMAB-moieties were in cis-conformation. The difference in fluorescence intensity is about 30% higher in case of trans-DMAB than in cis-DMAB (FIG. 12F). This difference in fluorescence intensities further indicates that the DMAB-modified motif 4 is destabilized by irradiation with UV-light thereby releasing DxR.

Next, we evaluated the percentage of DxR bound to motif 4. A fixed amount of motif 4 (5 μM) intercalated with a 10-fold excess of DxR for 12 h followed by a purification step using spin filtration. After each centrifugation step, a UV/Vis-spectrum of the flow through washing was recorded. A 20% reduction in DxR absorbance confirmed that approximately 8 equivalents of DxR intercalate per motif 4, and that 2 equivalents of excess DxR is removed through repeated washing (FIG. 14).

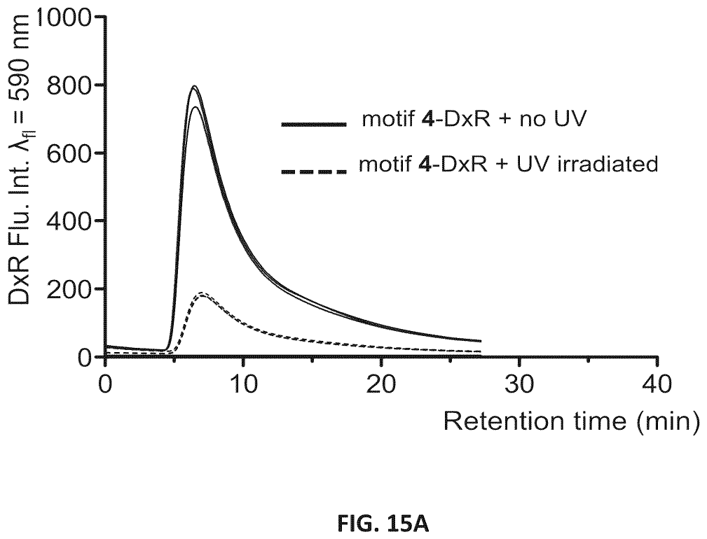

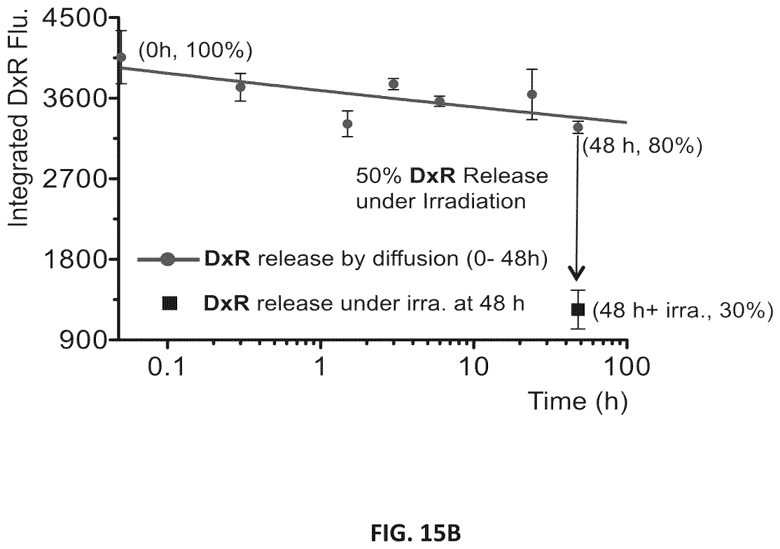

We then quantified the DxR release from the loaded DxR-L4 motifs under photoirradiation by an HPLC assay, detecting the fluorescence of the remaining DxR bound to motif 4 after removing unbound excess DxR from the solution. Phenol/CHCl₃ (1:1) washing is known to remove unbound excess DxR in the presence of DNA duplexes without removing the intercalated DxR. We then compared the amount of released DxR to that observed by self-diffusion of DxR into the buffer medium incubated at 37° C. over time (FIGS. 15A-B). After 5 min of UV irradiation (λ=365 nm, 350 mW/cm2), an approximately 3-fold drop in fluorescence emission was observed for the irradiated sample compared to the non-irradiated sample. Thus. UV irradiation triggered a rapid release of 63% of the encapsulated DxR (FIG. 15A). In contrast, a non-irradiated sample incubated at 37° C. released only about 20% of the loaded DxR from motif 4 over 48 h of incubation, due to thermal self-diffusion (FIG. 15B). To compare the UV-induced DxR release to thermally driven DxR diffusion at a fixed time interval, aliquots of sample incubated at 37° C. for 48 h were analyzed before and after irradiation with 365 nm UV light for 5 min. The release of DxR was monitored by measuring the fluorescence of irradiated vs. non-irradiated sample at 590 nm using a fluorescence detector attached to HPLC. DxR-loaded motif 4 incubated at 37° C. without UV exposure led to a release of 20% of the loaded DxR within 48 h of incubation by thermal self-diffusion. The same sample, however, released an additional 50% of the loaded DxR immediately after UV irradiation (FIG. 15B, black square). These results show that UV irradiation stimulated release of DxR from the motif 4.

### Example 7: Lipid-Mediated Self-Assembly of Motifs 3 and 4 Forms HyApNc

### 7.1. FRET Efficiency of Assembled Particles with both D (a550-DxR-L4) and A (a647-trCLN3-L4) Motifs

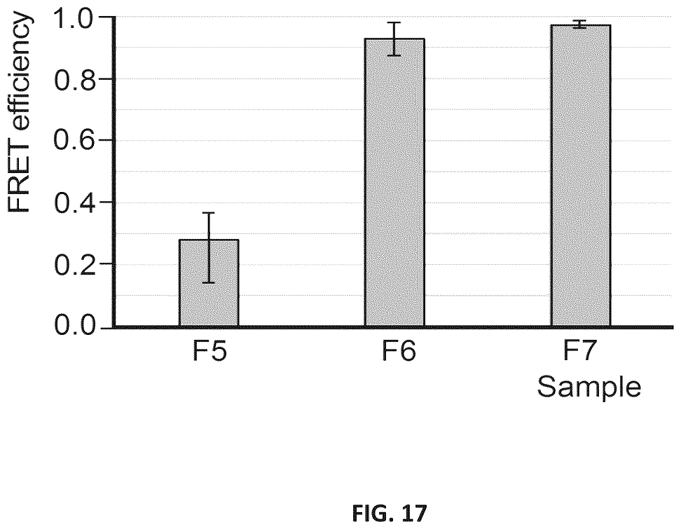

We performed steady-state fluorescence measurements on a Fluoromax 3 fluorometer (Horiba Jobin-Yvon) at 25° C. Fluorescence was excited at 554 nm (excitation of Atto550) and 644 nm (excitation of Atto647N), the entrance and exit slits were set to 5 nm, and integration time was set to 0.5 s. Apparent experimental FRET efficiencies were calculated using the direct method through E=(I_{A}/q_{A})/(I_{A}/q_{A}+I_{D}/q_{D}), where I_{A} is the acceptor peak fluorescence intensity after donor excitation from which contribution from donor fluorescence was subtracted. In is the donor peak fluorescence intensity after donors excitation, and the values for q_{A} (0.65) and q_{D} (0.8) are quantum yields of Atto647N and Atto550 dyes, respectively. The calculation of FRET efficiency for the atto dyes are not fully determined and our calculation is a good approximation of changes in the distances. This calculation does not provide absolute values of distance between the dyes, however, it is an effective way to determine relative changes in distance between the fluorophores. Nanoconstructs assembled with motifs Atto647-3 and Atto550-4 (HyApNc) yielded a FRET efficiency of 92% as compared to 27% where both motifs 3 and 4 lack the lipid modifications (F6 vs. F5). When a non-cMet-binding Atto647N-labeled mutant trCLN3-14 motif (Atto647mut-3) was used instead of Atto647N-3, the resulting mutated nanoconstruct HyApNc.mut yielded a similar FRET efficiency (97%) as shown by HyApNc (F7 vs. F5). These results show that both motifs properly assemble in presence of 5′-lipid modification to form hybrid nanoconstructs as compared to the non-lipidated motifs. See FIG. 17.

### 7.2. Stability of HyApNe Micellar Nanoconstruts in Presence of Human Blood Serum (HBS) and Bovine Serum Albumin (BSA)

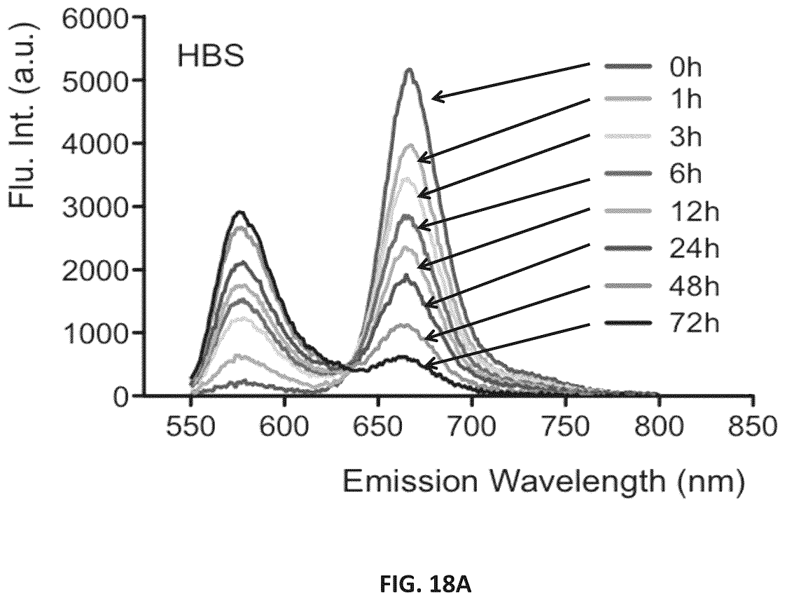

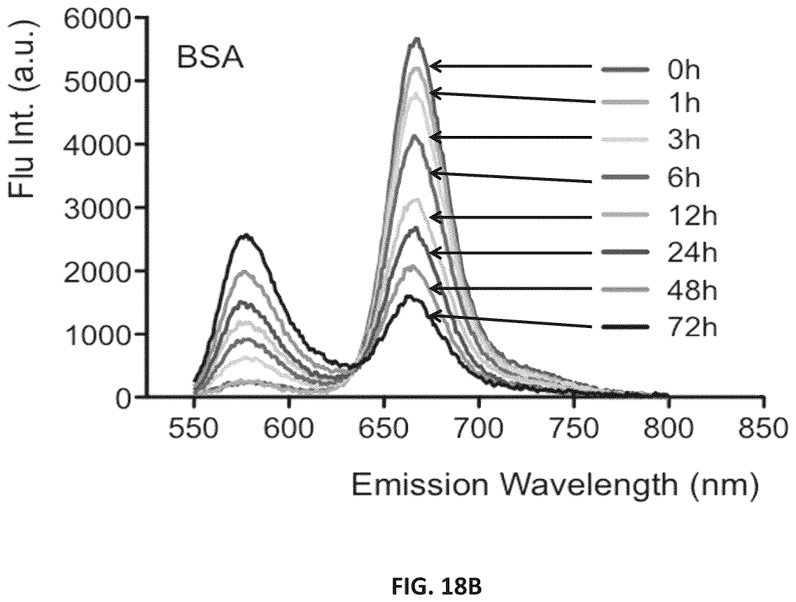

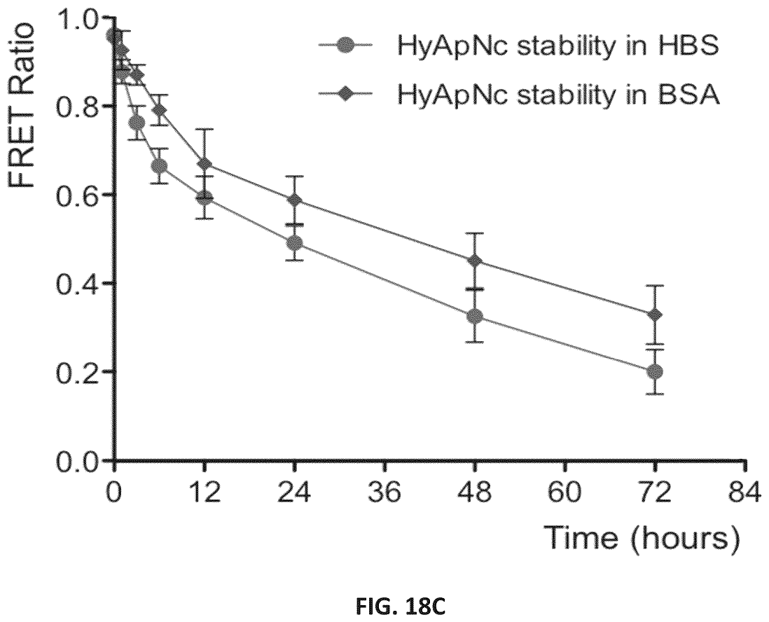

The integrity of the micellar nanoconstruts HyApNe was tested in a FRET assay in presence of human blood serum (HBS) and in bovine serum albumin (BSA) solution. See M. Kastantin et al., J. Phys. Chem. B. 2010, 114, 12632-12640; H. Dong et al., J. Am. Chem. Soc. 2012, 134, 11807-11814; which references are incorporated herein in their entirety. A suitable FRET pair Atto-647N-3 as the acceptor and Atto550-4 as the donor was used to assess the stability of micelles in the presence of 95% HBS and 1 mM BSA solution. In a FRET experiment, 2 μM of HyApNc containing the FRET pair (Atto647-3 & Atto550-4) in 1:1 ratios were incubated with 95% human blood serum and 1 mM BSA solutions separately at 37° C. For each measurement an aliquot of 20 μl samples were taken after indicated time intervals of 0, 1, 3, 6, 24, 48 and 72 h respectively, transferred into a 384-well plate and the time-resolved fluorescence spectra of FRET pairs were measured by using an excitation wave length of λₑₓ=535 nm and an emission wavelength spectrum between λ=550 nm and λ=800 nm was recorded using an EnSpire® Multimode Plate Reader (PerkinElmer). The FRET ratio was calculated by using the equation FRET ratio=I₆₆₉/(I₆₆₉+I₅₇₆) which, yields the relative stability of the micelles. The approximate half-life of the HyApNc was estimated to be (t_{1/2}) of 14 hours in 95% human blood serum and 18.0 hours in 1 mM BSA solution respectively. The FRET ratios show a decrease in the FRET efficiency over time indicating that the micellar nanoconstructs gradually disassembled over a period of 72 h. See FIGS. 18A-C.

### 7.3. Results

We next combined both lipid-modified motifs 3 and 4 to test their lipid-mediated self-assembly into heterogeneous HyApNc. By mixing free Atto-647N-trCLN3-L4 (Atto647N-3) with Atto550-labeled DxR-L4 motif (Atto550-4) in different ratios, hybrid nanoconstructs were formed and stabilized by the strong hydrophobic interaction of the lipid tails. The Atto-dye labels were attached at the 5′-end in immediate proximity to the lipid-modifications to ensure that intermolecular FRET effects report the formation of micellar nanoconstructs. In the FRET experiment nanoconstructs self-assembled by mixing a fixed concentration of 5 μM Atto647N-3 with Atto550-4 in concentrations ranging between 1-15 μM (Table 4).

TABLE 4
Concentrations [μM] of Atto-labeled motifs 3 and 4 mixed in different ratios to form
hybrid micellar nanoconstructs

| | | | | | I₆₆₉^{a} | I₅₇₆^{b} | |
|---|---|---|---|---|---|---|---|
| | Atto550-4 | Atto647N- | volume | Equivalents | [mean ± | [mean ± | |
| Exp. No. | [μM] | 3 [μM] | (μL) | Atto550-4 | sd] | sd] | I₆₆₉/I₅₇₆^{c} |
| 1 | 0.0 | 5.0 | 20 | 0.0 | 652 ± 206 | 41 ± 7 | 15.90 |
| 2 | 1.0 | 5.0 | 20 | 0.2 | 2317 ± 657 | 416 ± 116 | 5.56 |
| 3 | 1.75 | 5.0 | 20 | 0.35 | 5673 ± 881 | 775 ± 169 | 7.32 |
| 4 | 2.5 | 5.0 | 20 | 0.5 | 9604 ± 1172 | 1218 ± 234 | 7.88 |
| 5 | 5.0 | 5.0 | 20 | 1.0 | 21098 ± 402 | 3553 ± 434 | 5.93 |
| 6 | 7.5 | 5.0 | 20 | 1.5 | 28225 ± 1164 | 6106 ± 378 | 4.62 |
| 7 | 10 | 5.0 | 20 | 2.0 | 34010 ± 3593 | 9992 ± 153 | 3.40 |
| 8 | 15 | 5.0 | 20 | 3.0 | 35242 ± 5951 | 27766 ± 4606 | 1.26 |

^{a}Fluorescence intensities at λ = 669 nm.
^{b}Fluorescence intensities at λ = 576 nm.
^{c}Estimated ratio (I₆₆₉/I₅₇₆) from the FRET experiments.

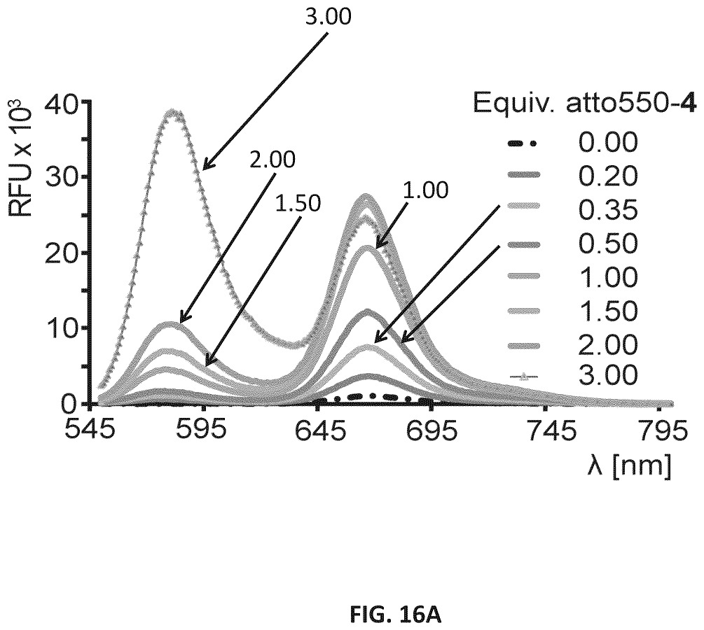

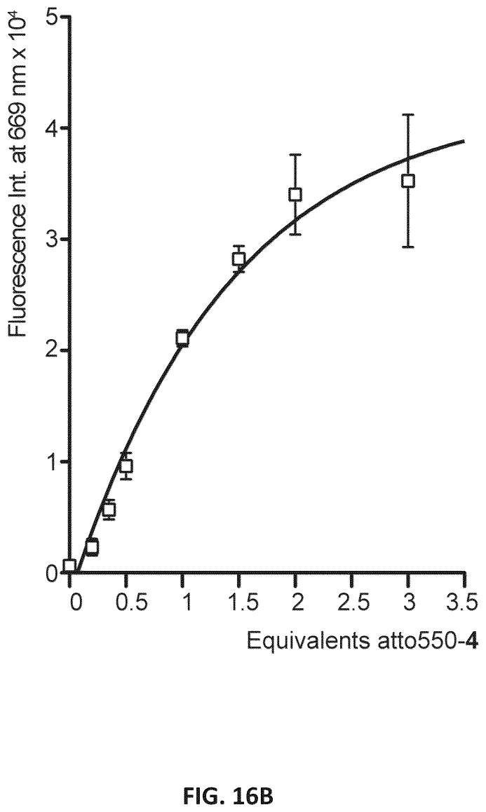

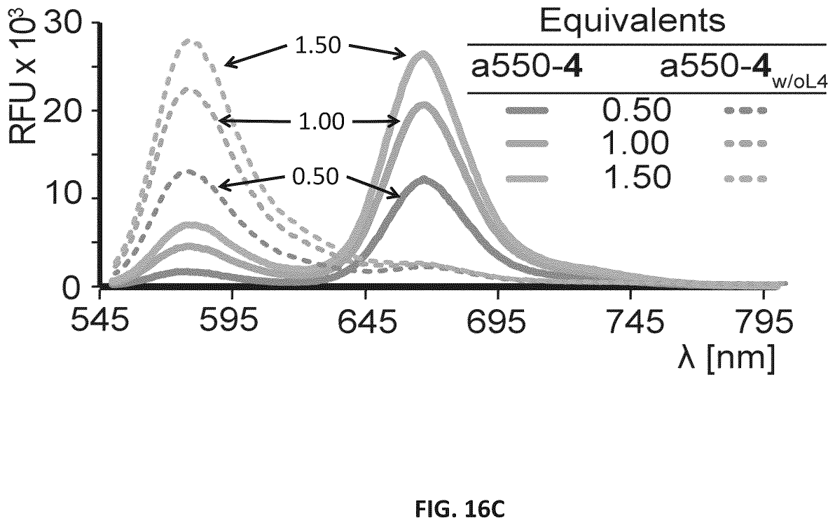

Fluorescence at λₑₓ=535 nm (FIG. 16A) showed that the nanoconstructs self-assembled with 0.2 equivalents of Atto550-4 (Atto647N-3: Atto550-4=5:1), yielding an intensity ratio I₆₆₉/I₅₇₆ of 5.56. In contrast, nanoconstructs self-assembled with 0.35 or 0.5 excess equivalents of Atto550-4 showed an increasing I₆₆₉/I₅₇₆ value of 7.32 and 7.88, respectively, a significant enhancement of ˜32% and ˜41% relative to the Atto647N fluorescence. An increase in FRET observed with increasing concentrations of Atto550-4 reached saturation between 2.0 and 2.5 equivalents (FIG. 16B). Nevertheless the I₆₆₉/I₅₇₆ value already reaches 5.93 at one equivalent of Atto-550-4 (Atto647N-3:Atto550-4=1:1). Therefore, we maintained this ratio in the subsequent cellular studies to achieve a proper balance between high target affinity (internalization efficiency) and DxR carrying efficiency (cytotoxicity).

In a control experiment, we employed the Atto550-labeled DxR-binding motif without lipid modification (a550-4_{w/oL4}). With this lipid-devoid motif, only diffusion-controlled encounters between Atto550 and Atto647N can occur, which should result in low relative intensities. Indeed, with a 1:1 ratio of 3 and Atto550-4_{w/oL4} we observed an I₆₆₉/I₅₇₆ value of 0.09, indicating that no hybrid micellar nanoconstructs are forming (FIG. 16C). The FRET-signal thus strictly depends on the ratio of the two functional domains and on the presence of the L4-modification. A comparison of FRET efficiency values (see above; FIG. 17) suggested the 92% FRET efficiency for assembled HyApNc consisting of motifs Atto550-4 and Atto647-3 as compared to 27% where both motifs 4 and 3 lack the lipid modifications. When a non-cMet-binding Atto647N-labeled mutant trCLN3-14 motif (Atto647mut-3) was used instead of Atto647N-3, the resulting mutated nanoconstruet HyApNe.mut yielded a FRET efficiency (97%), similar to HyApNc. Together, these data provide evidence that both motifs self-assemble to form hybrid heterogeneous nanoconstructs of spherical geometry when the lipid modifications are present. The FRET signal intensity is also a good measure of integrity of the nanoconstructs.

The resulting HyApNc consisting of 3 and 4 in a 1:1 ratio was further analyzed by AFM to compare its size and structural features with nanoconstructs resulting only from motif 3. We observed that the hybrid micellar nanoconstruct retained its spherical shape similar to the homogenous nanoconstructs consisting of only motif 3 (see FIG. 8B). However, their average diameter is 32.3±2.1 nm—larger than the homogenous nanoconstructs made from trCLN3-L4 (motif 3), which averaged 21.2±1.5 nm (FIG. 8C). Without being bound by theory, the increased size of the heterogenous nanoconstructs as compared to the homogenous constructs may result from differences in the physico-chemical properties of the two aptamers in 3 and 4, from structural differences, or both.

Cell internalization and delivery of the intercalated DxR to the target cells may depend on the integrity of the micellar nanoconstruts over time. The stability of the micelles as well as their circulation time can be affected by the presence of serum proteins, which may alter the micellar equilibrium leading to their dissociation to varying extents. Therefore we evaluated the integrity of HyApNc upon interaction with human blood serum (HBS), and in presence of bovine serum albumin (BSA) at 37° C. over time (see Example 7.2; FIGS. 18A-C). We assessed the integrity of the micellar nanoconstruct HyApNc by using the previously assembled FRET pair (see FIG. 16) attached to the 5′-ends of both motifs 3 (Atto647N-3) and 4 (Atto550-4). The intermolecular FRET effect was monitored (FIG. 18A, B) and an increase in the fluorescence intensity at 576 nm and a decrease at 669 nm was observed over time. This result indicates that the micellar nanoconstructs disintegrate gradually in the presence of BSA or serum proteins contained in HBS. The FRET ratio=I₆₆₉/(I₆₆₉+I₅₇₆) was calculated and plotted as a function of time (FIG. 18C). The HyApNc nanoconstructs exhibited a half-life (t_{1/2}) of 14 hours in 95% HBS and of 18hours in 1 mM BSA solution. The time-resolved emission data indicate that the rate of micellar nanoconstruct disintegration in either BSA or HBS was not significantly different. The t_{1/2} indicates an adequate stability of the micelles in blood serum with slow disintegration under our in vitro experimental conditions. If necessary for certain applications, the half-life of HyApNc could be further increased. For example, stability may be increased by elongating the lipid chains and/or by using unsaturated lipids and crosslinking them at the core of the nanostructures.

### Example 8: Cellular Uptake of Aptameric Nanoconstructs by cMet Expressing Cells

### 8.1. Flow Cytometry Analysis

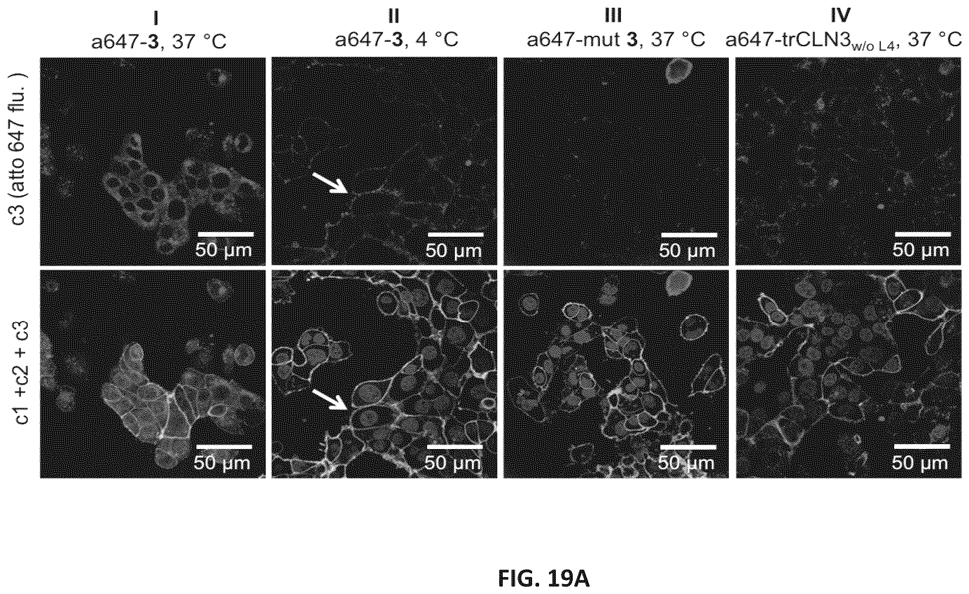

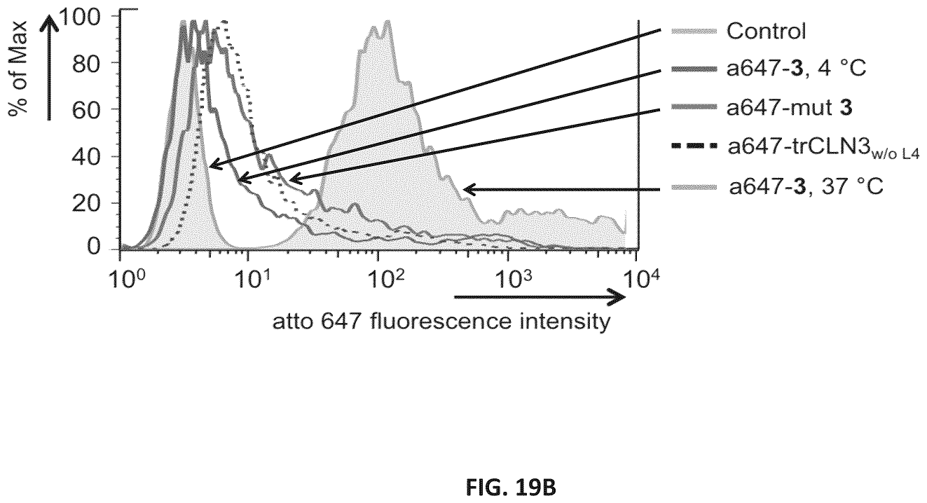

For analysis of trCLN3 internalization using flow cytometry, approximately 1×10⁵ NCI-H1838 cells/well were seeded in a 24-well plate and incubated for 24 h at 37° C. After 24 hours of incubation, the cells were washed with 200 μL of 1×PBS and then incubated with 200 μL of 1 μM Atto 647 labeled aptamer motifs i) a647-3 at 37° C. ii) a647-3 at 4° C. iii) a647-mut 3 at 37° C. and iv) a647-trCLN3_{w/oL4} at 37° C., respectively, for 2 h. The cell medium was removed and the cells were detached from the plates using trypsin-EDTA and transferred to FACS tubes. The cells were then washed twice by centrifugation with 0.5 mL buffer and the cell pellets were resuspended in 100 μL of 1×PBS buffer and subjected to flow cytometric analysis using a BD FACS Canto™ II Flow Cytometer (BD Biosciences). Fluorescence emissions from Atto-647 labeled aptamer motifs were collected with a 660/20-nm band-pass filter. See FIG. 19B. A minimum detection of 10,000 events were collected and analyzed with the FlowJo software program.

For flow cytometry analysis of HyApNc-mediated DxR uptake, the H1838 cells (1×10⁵ cells/well) were seeded for 24 h at 37° C. The cells were washed with 1×PBS (200 μL) and subsequently treated with i) free DxR (as control), ii) targeted nanoconstructs HyApNc-DxR or iii) mutated non-targeted nanoconstructs HyApNc.mut-DxR or iv) HyApNc_{w/oAz}-DxR with a final DxR concentration of 8 μM in the culture medium. The plates were then incubated for 2 h at 37° C. Afterwards, the cells were detached from the plates by trypsinization and transferred to FACS tubes. The cells were then washed twice by centrifugation with 0.5 mL buffer. Afterwards the cell pellets were resuspended in 100 μL 1×PBS buffer and cither irradiated with UV light for 5 minutes (_=365 nm, 350 mW/cm²) or not irradiated before subjected to FACS analysis. Fluorescence emissions of the internalized DxR were recorded with a 585/42-nm band-pass filter.

### 8.2. Results

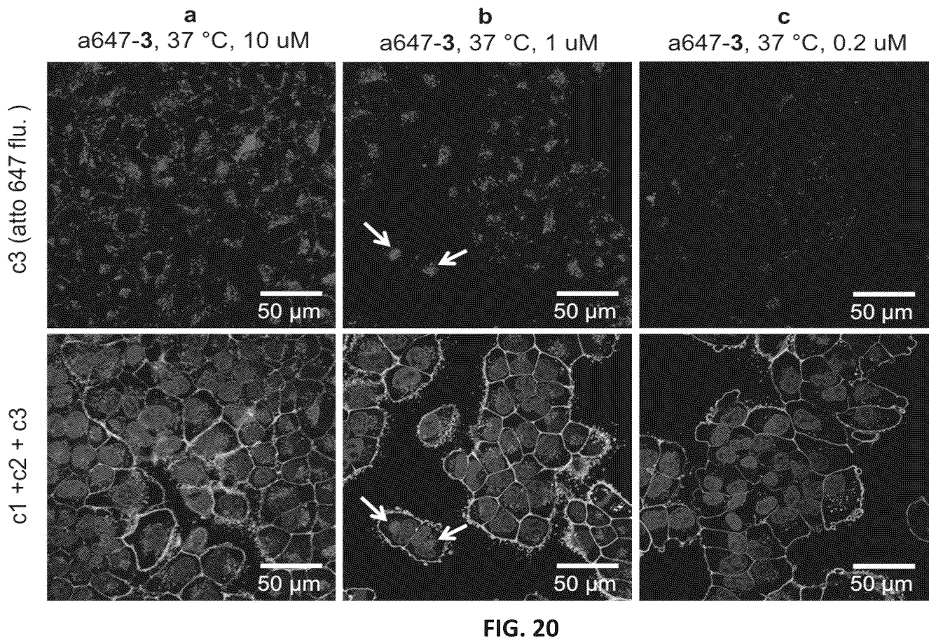

After confirming formation of the aptameric nanoconstructs, the cell targeting ability and internalization efficacy of aptamer trCLN3-L4 (3) mediated by cMet recognition was investigated using both confocal microscopy and flow cytometry analysis. Cell uptake experiments were performed with the NCI-H1838 lung cancer cell line that expresses high levels of cMet. NCI-H1838 cells incubated with different concentrations of the Atto647N-3 (10 and 1 μM, respectively) at 37° C. for 90 min, showed a strong and comparable intracellular red-fluorescence at both concentrations above the CMC value (FIG. 19A, I for 10 μM and FIG. 20(b) for 1 μM). At 1 μM of Atto647N-3, a punctuated pattern of internalized nanostructures was observed in the cytoplasm, suggesting that they may localize in endosomes (FIG. 20(b)). Indeed, the same experiment performed at 4° C. showed only a weak membrane-localized fluorescence (FIG. 19A, II) with markedly reduced Atto647-fluorescence in the H1838 cells, consistent with inhibition of endocytosis at low temperature. When the Atto647N-3 concentration was reduced to 0.2 μM, which is below the CMC, a significantly weaker fluorescence signal was observed, as expected (FIG. 20(c)).

H1838 cells incubated with 5′-Atto647N-labeled double mutant of 3 (Atto647N-mut 3) that docs not bind to cMet exhibited marginal cellular staining (FIG. 19A, III), consistent with lack of internalization. Finally, the non-lipidated version of Atto647N-trCLN3_{w/oL4} also showed low cellular staining (FIG. 19A, IV), suggesting that lipidation of the cMet-binding aptamer is required for efficient uptake. This result suggests that protein target binding in solution could differ from targeting the protein at the cell surface. Moreover, lipidation of aptamers potentially improves their ability to target proteins expressed on cell surfaces by self-organizing multiple aptamers in a single nanostructure, although the generality of this notion remains to be demonstrated with other aptamer/target systems.

These findings were further confirmed through flow cytometric studies (FIG. 19B, Example 8.1). There was a noticeable change in the fluorescence signal observed for cells treated with free Atto647N-trCLN3_{w/oL4} (FIG. 19B, dotted line) compared to the auto-fluorescence profile of untreated cells (FIG. 19B, “Control”), indicating low internalization. In comparison with non-lipidated Atto647N-trCLN3_{w/oL4}, cells treated with Atto647N-3 at 37° C. (FIG. 19B, “a647N-3, 37° C.”) showed significantly higher shift in fluorescence intensity. A minimal shift in fluorescence intensity was also observed for cells treated with cither Atto647N-mut 3 (FIG. 19B, “a647-mut 3”) or Atto647N-3 at 4° C. (FIG. 19B, “a647N-3, 4° C.”) over untreated cells (FIG. 19B, “Control”), indicating either a low non-specific binding or only a membrane localized binding without internalization at low temperature. Taken together, these results show that uptake into H1838 cells can depend upon: i) the ability to bind extracellular cMet by the aptamer moieties; ii) the ability to form nanoconstructs due to lipidation; and iii) that the uptake is temperature-dependent, supporting an endocytotic mechanism. Such features can be adjusted to affect desired properties.

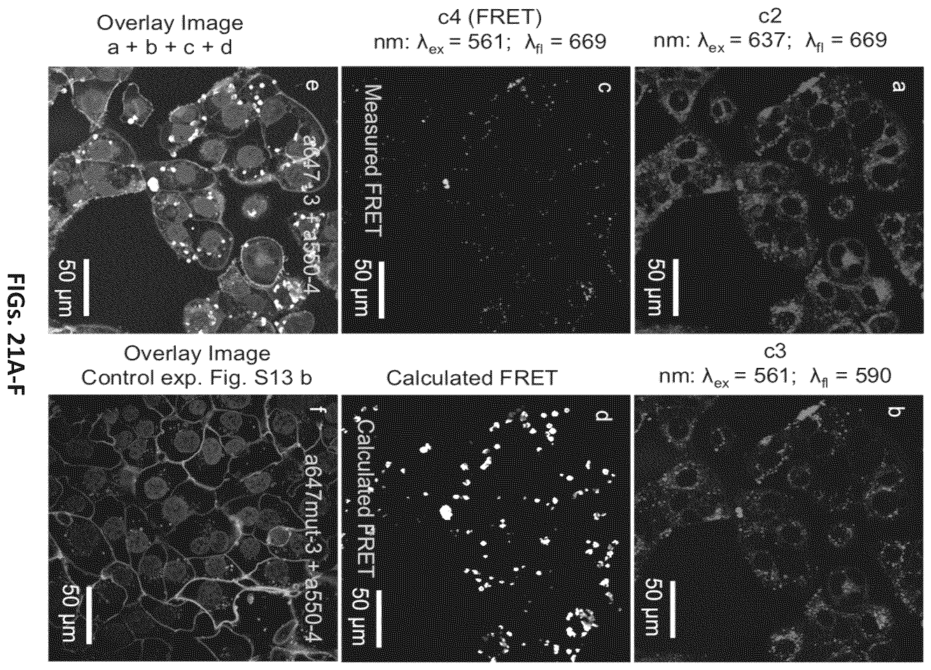

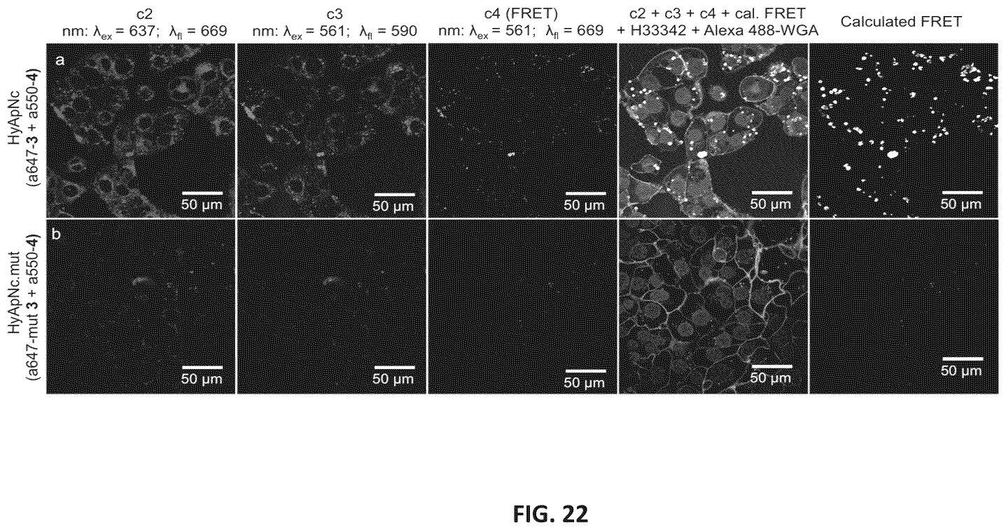

We next performed cellular uptake studies of a dual-labeled hybrid-nanoconstruct (HyApNc) containing a mixture of Atto550-labeled 4 and Atto647N-labeled 3 motifs in a 1:1 ratio. Both fluorescent probes constitute a suitable FRET pair entrapped within the lipid core that can be employed to validate whether the functional nanoconstructs enter and target H1838 cells. The confocal images showed not only the cellular staining for both dyes (FIG. 21A; c2, FIG. 21B; c3, cellular shapes), but also a FRET signal (FIG. 21C; c4, white regions) was observed. During confocal imaging all settings were kept constant (for details see Example 1). To evaluate the occurrence of FRET, we analyzed the images using a method that was previously reported (Carlo, D. S. & Harris, J. R. Negative staining and Cryo-negative Staining of Macromolecules and Viruses for TEM. Micron 1993, 42, 117-131, which reference is incorporated by reference herein in its entirety), where the PixFRET plugin of the image processing software ImageJ was used for FRET quantification. Briefly, the bleed-through of the acceptor and donor channels was determined and finally the calculated FRET images were reconstructed (FIG. 21D; calculated FRET). The calculated FRET images suggest donor and acceptor dyes are in correct geometry, supporting the integrity of the nanoconstructs. High FRET efficiencies were only observed when the designated constructs were able to enter the cells (FIGS. 21A-C). FIG. 21E shows the overlay of the images shown in FIGS. 21A-D. In contrast, mutated nanoconstructs (HyApNc.mut) containing the non-cMet-binding Atto647N-labeled mutant trCLN3-L4 motif and Atto550-labeled motif 4 resulted in poor FRET efficiencies (FIG. 21F, FIG. 22), similar to background signals, indicating that the process of internalization is target-specific rather than occurring randomly.

### Example 9: Photo-Triggered Release of DxR from HyApNc-DxR

### 9.1. Time Dependent UV Exposure on Cell Mortality

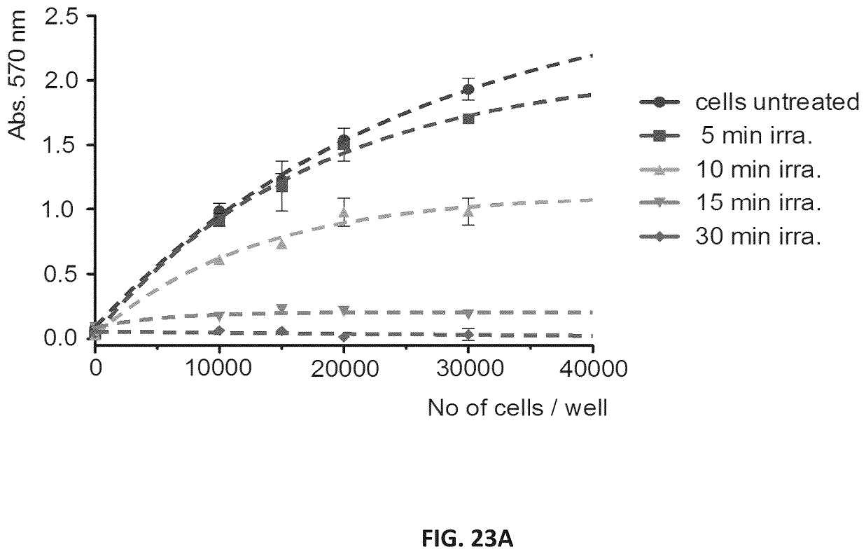

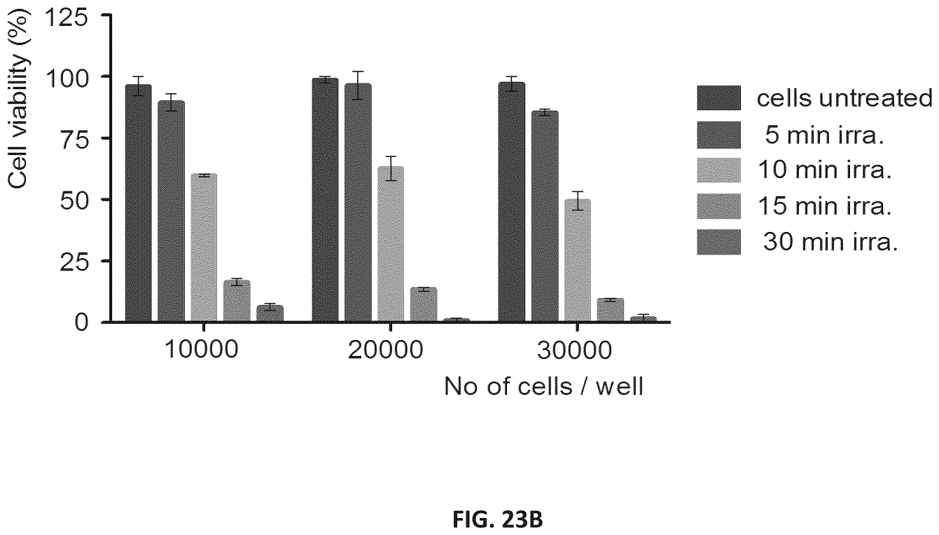

In order to test the influence of time dependent UV exposure on cell mortality, H1838 cells were grown at different seeding densities of 10,000, 15,000, 20,000 and 30,000 cells per well in duplicates in a 96-well plate 24 hours prior to the experiments. After 24 hours of incubation at 37° C. in 5% CO₂-atmosphere, the cell medium was replaced with 100 μL of fresh RPMI medium. Each well containing a different cell density was exposed to UV irradiation of 365 nm for 0, 5, 10, 15 and 30 minutes respectively at a fixed intensity of 350 mW/cm² and the cells were allowed to grow further for 24 hours. Afterwards 10 μL of an MTT stock solution (5 mg/mL) was added to each well and the cells were incubated at 37° C. for 6 hours. After labeling the cells with MTT, 100 μL of the SDS-HCL solution were added to each well and mixed thoroughly by use of a pipette and incubated at 37° C. for an additional 12 hours. Finally the absorbance was measured at λ=570 nm by using a Tecan Infinite® M1000 PRO microplate reader. The percentage of cell viability was determined by comparing the UV treated cells with the untreated control samples. See FIGS. 23A-B.

### 9.2. Results

After successfully targeting the H1838 cells with HyApNc, we further investigated the selective transport of DxR into the cells, followed by its light triggered release from the HyApNc. The DxR-loaded HyApNc (HyApNc-DxR complex) was prepared by mixing motif 3 and 4 (1:1 ratio) with 10-fold excess of DxR followed by a purification step using spin filtration (details are given in Example 1). To ensure minimum cell mortality upon UV-irradiation, H1838 cells were irradiated at t=0, 5, 10, 15 and 30 minutes, respectively, at an intensity of 350 mW/cm². Cell viability as a function of time dependent response to UV treatment was measured by an MTT assay 24 h after irradiation. A maximum survival rate comparable to the non-irradiated control (t=0 min) was observed at an irradiation time t≤5 min (FIGS. 23A-B).

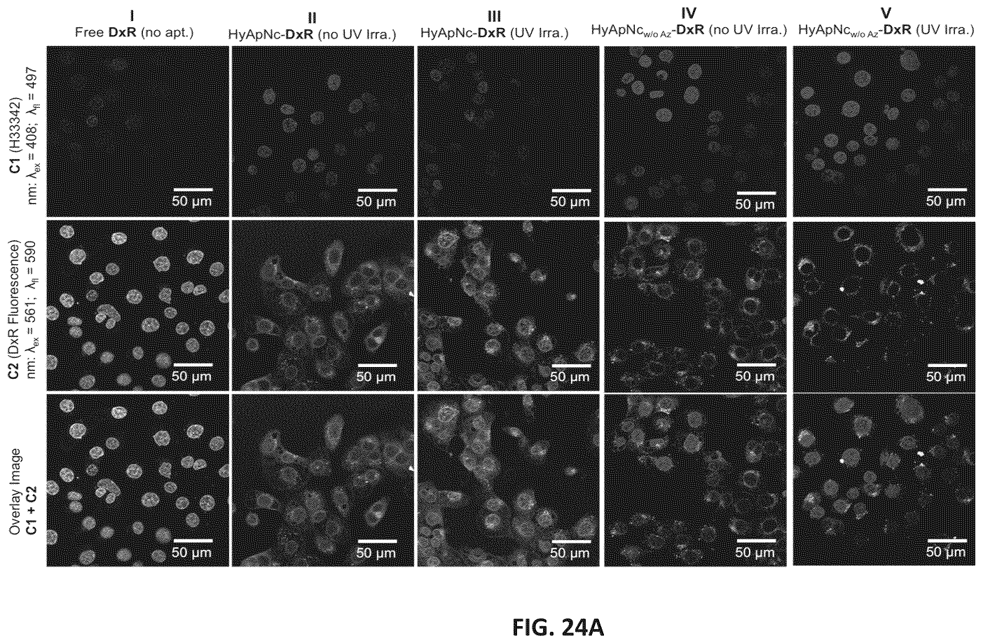

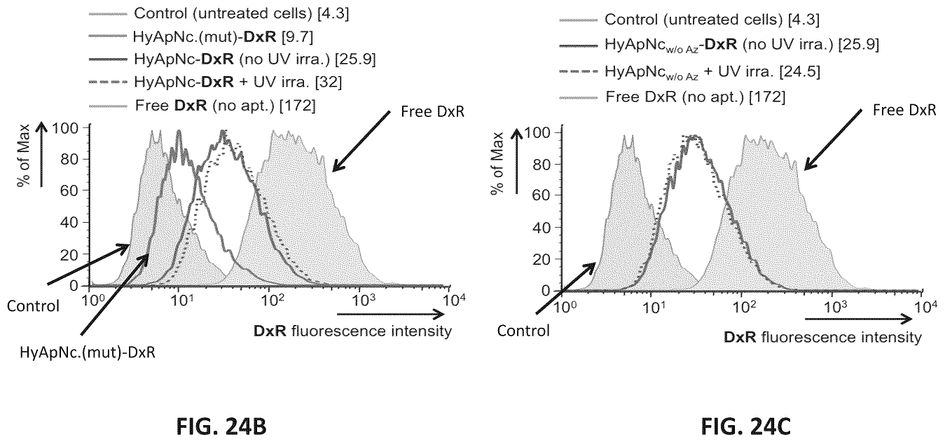

To verify the HyApNc-mediated selective transport of DxR to target cells and its light-triggered release from motif 4, we monitored the fluorescence signal of DxR within and outside of the cell nuclei of H1838 cells that were treated with either free DxR (as control) or with HyApNc-DxR (see Example 1 for details of DxR loading), while keeping the DxR concentrations in the bound and the unbound form fixed at 40 μM (5 μM HyApNc carrying 8 equivalents of DxR). The release of DxR from HyApNc was investigated by confocal microscopy with and without subsequent irradiation at 365 nm. Confocal images of the H1838 cells at 37° C. after 2 h of incubation showed a decrease in the DxR fluorescence signal in the cell nuclei in the following order: free DxR, HyApNc-DxR complex with and without UV irradiation (λ=365 nm, 350 mW/cm²) (FIG. 24A, I-III). Strong DxR fluorescence was observed in cell nuclei after treatment with free DxR, indicating that free DxR readily diffuses through the plasma membrane and accumulates almost exclusively in the nuclear region (FIG. 24A, I). However, the HyApNc-DxR complex without UV irradiation led to a considerably weaker DxR-fluorescence in the nucleus and a noticeable fluorescence within the endoplasm confirming that most of the DxR is predominantly localized outside the nucleus bound to the HyApNc (FIG. 24A, II). In contrast. when the HyApNc-DxR complex is exposed to irradiation (┘=365 nm, 350 mW/cm²) a discernible increase in both nuclear and extranuclear fluorescence was detected (FIG. 24A, III). When control experiments were performed with a construct lacking DMAB (HyApNc_{w/oAz}-DxR), near-identical DxR fluorescence signals are predominantly observed in the cytosol of the cells with and without UV exposure (FIG. 24A, IV-V). No visible increase in the DxR fluorescence signal was observed in either the nuclei or in the cytosol when the cells treated with HyApNc_{w/oAz}-DxR were irradiated (FIG. 24A, V) compared to non-irradiated cells (FIG. 24A, IV).

HyApNc-mediated DxR internalization with or without DMAB was further evaluated by flow cytometry. See Example 8.1 for methodology. As a control, the DxR uptake of the non-targeted mutated nanoconstruct HyApNc.mut-DxR was compared to that of the targeted nanoconstructs HyApNc-DxR. To accomplish this, H1838 cells were incubated with free DxR, HyApNc.mut-DxR, (HyApNc-DxR), or targeted nanoconstructs without DMAB (HyApN_{w/oAz}-DxR) at fixed DxR concentrations of 8 μM either in its free form or in its complex form with the carrier (1 μM of nanocarrier, each containing 8 eqivalents of DxR). Treatment of cells with free DxR (FIGS. 24B-C, areas labeled “Free DxR”) induces a 5-fold increase in mean cellular fluorescence intensity as compared to cells incubated with an equivalent dose of either HyApNc-DxR (FIG. 24B, central peak, solid line) or HyApNc_{w/oAz}-DxR (FIG. 24C, central peak, solid line). Instead, irradiation of cells treated with HyApNc-DxR (FIG. 24B, central peak, dotted line) induces only about a 1.3-fold shift in the fluorescence intensity compared to the non-irradiated cells (FIG. 24B, central peak, solid line). Without being bound by theory, this small shift in the fluorescence intensity might be due to the limitations of the flow cytometer to discriminate between the nuclear and the extranuclear fluorescence signal. In contrast, cells incubated with HyApNc_{w/oAz}-DxR showed a −1.05-fold shift in fluorescence intensity, and the FACS profile of the irradiated sample (FIG. 24C, central peak, dotted line) was comparable to the non-irradiated samples (FIG. 24C, central peak, solid line). Moreover, cells incubated with HyApNc-DxR exhibited a 2.8-fold increase in the mean fluorescence signal compared to cells treated with HyApNc.mut-DxR containing the same amount of DxR in either case (FIG. 24B, central peak, solid line vs. line labeled “HyApNc.(mut)-DxR”). This result showed that non-targeted nanoconstructs HyApNc.mut-DxR exhibited significantly lower efficacy in DxR delivery. consistent with their lower level of cellular uptake compared to HyApNc-DxR observed in FIGS. 21A-F. This result indicates that after UV irradiation, most of the intercalated DxR was released from HyApNc having DMAB units and subsequently transferred into the nuclei and co-localized with the Hoechst dye.

### Example 10: In Vitro Cytotoxicity of HyApNc-DxR against NCI-H1838 Cells

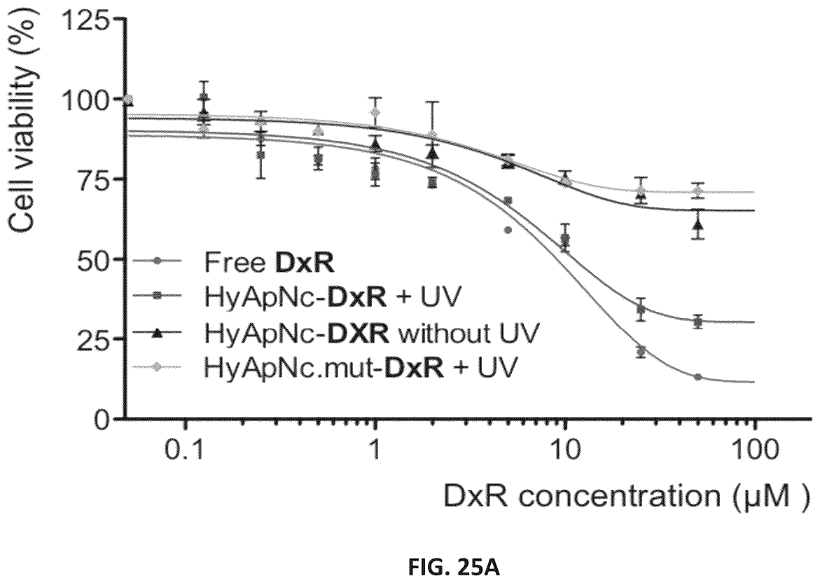

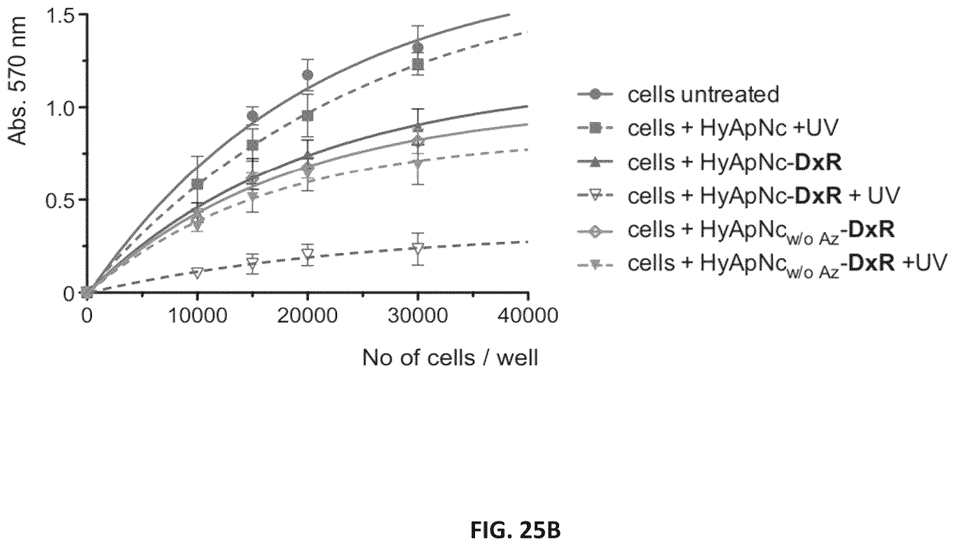

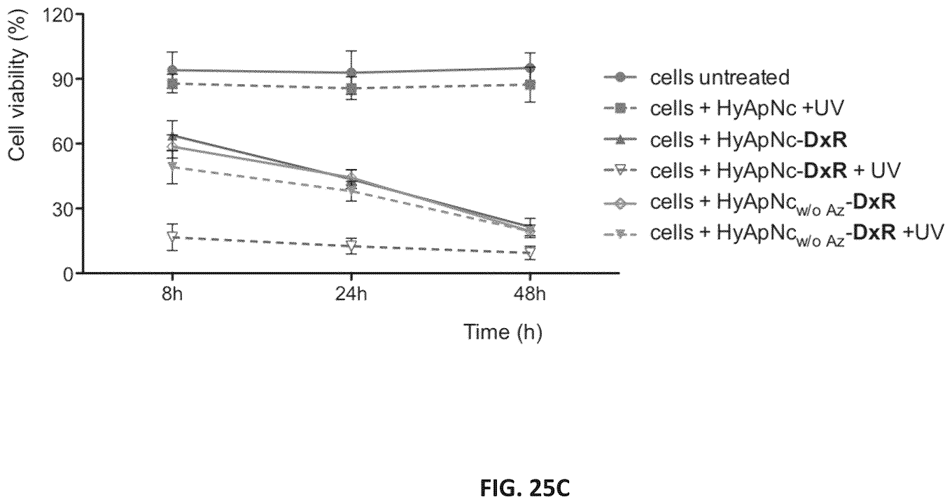

Having shown that the DxR can be selectively transported into target cells, we evaluated the cytotoxicity of the free DxR, the HyApNc-DxR, and the non-targeting HyApNc.mut-DxR nanoconstructs with and without UV irradiation in H1838 cells by an MTT assay (details see Example 1) in a dose dependent way between 0.125 μM and 50 μM (FIG. 25A). There was a clear dependence of the H1838 cell viability on the concentration of DxR (FIG. 25A). An IC₅₀ of 11 μM (6.5 μg/mL) was determined for HyApNc-DxR irradiated with UV light (FIG. 25A, ▪), and a similar level of cytotoxicity (IC₅₀=8 μM (4.7 μg/mL)) was observed for free DxR (FIG. 25A, ●). However, no significant cytotoxicity was measured when cells were either treated with HyApNc-DxR without UV (FIG. 25A, ▴) or with HyApNc.mut-DxR (FIG. 25A, ♦). Cells incubated with non-targeting HyApNc.mut-DxR with subsequent UV irradiation under the same conditions (FIG. 25A, ♦) exhibited about a 38% increase in cell survival compared to cells treated with HyApNc-DxR at 8 μM loaded DxR concentrations (FIG. 25A, ♦ vs. ▪), consistent with their lower level of cellular uptake compared to HyApNe-DxR observed in FIGS. 21A-F. Without being bound by theory, this result suggests that the cMet-overexpressing H1838 cells effectively internalized HyApNc-DxR due to receptor-mediated endocytosis, while non-targeted nanoconstructs exhibited significantly lower efficacy.

As an additional control. we conducted a time dependent cytotoxicity assay to determine whether DxR release would occur solely through self-diffusion after endocytosis (i.e. no UV radiation). To accomplish this. we used the DMAB lacking construct (HyApNc_{w/oAz}-DxR) at different incubation times. H1838 cells were treated with (i) unloaded HyApNc (ii) HyApNc-DxR, and (iii) HyApNp_{w/oAz}-DxR for 2 h at 37° C. at 8 μM DxR dosage. After 2 h post-treatment, the cells were washed, the RPMI medium replaced with fresh medium, and some of them (FIG. 25B, dotted lines) were exposed to UV light for 5 minutes (┘365 nm; 350 mW/cm²), while those that were not irradiated were used as controls (FIG. 25B, solid lines). Afterwards cells were further allowed to incubate at 37° C. for 8 h, 24 h, and 48 h, respectively before being subjected to the MTT assay. Cells treated with only RPMI medium and not exposed to UV-irradiation (FIG. 25B, ●) served as the primary control.

Cells treated with HyApNc alone in combination with UV irradiation exhibited similar survival rates as non-irradiated cells treated with only RPMI medium (FIG. 25B, ▪vs. ●), indicating that neither the nanoconstruct without DxR nor brief UV exposure contribute significantly to cell death. In contrast. the combination of HyApNc-DxR with UV irradiation induced an approximately 2.8-fold decrease of cell viability compared to the treatment with HyApNc-DxR alone (17% vs. 64%) 8 h post treatment (FIG. 25B, unfilled ▾ vs. ▴). When cells were treated with the photo-deactivated construct HyApNc_{w/oAz}-DxR in combination with UV light a 0.2-fold decrease of cell viability compared to non-irradiated HyApNc_{w/oAz}-DxR (49% vs. 59%) was measured (FIG. 25B, ▾ vs. ⋄). This result indicates that the lower cell mortality is related to inefficient release of DxR from the nanoconstrct without DMAB phtoswitches. We further evaluated cell viability for the incubation times of 24 and 48 h under similar conditions as for the 8 h incubation. Cells incubated with HyApNc-DxR without UV irradiation (FIG. 25C, ▴) showed a gradual decrease in viability from 64% (8 h) to 43% (24 h) to 21% (48 h). Cells incubated with HyApNc_{w/oAz}-DxR under the same conditions (FIG. 25C, ⋄) decreased from 59% (8 h) to 44% (24 h) to 19% (48 h). When UV irradiation was applied to the HyApNc_{w/oAz}-DxR-treated cells (FIG. 25C, ▾), cell viability was similar. Thus, a clear differentiation between 5 min UV-irradiation of HyApNc-DxR and all other conditions was seen for the 8 h and 24 h incubation times whereas at 48 h incubation cells were killed equally efficient under all conditions that contained DxR. At 48 h, a sufficient amount of intercalated DxR might have diffused from the control-nanoconstructs or the non-UV irradiated ones spontaneously, and induce cell killing equally efficiently. For UV-irradiated HyApNc-DxR, a ˜80% cell mortality is already achieved within a significantly shorter time-span of 8 h (FIG. 25C, unfilled ▾).

Although preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A nucleic acid-based assembly comprising:
(a) at least one nucleic acid aptamer;
(b) at least one nucleic acid motif designed to physically capture a drug, wherein the nucleic acid motif comprises one or more photo-responsive moieties that effect the release of the drug upon irradiation; and
(c) at least one lipid.

2. The nucleic acid-based assembly according to claim 1, wherein the at least one aptamer and the at least one nucleic acid motif each are covalently linked to at least one lipid, wherein the lipid-modified aptamer and lipid-modified nucleic acid motif form the assembly through noncovalent interaction.

3. The nucleic acid-based assembly according to claim 1 or claim 2, wherein the at least one lipid comprises a triglyceride, diglyceride, monoglyceride, fatty acid, steroid, wax, or any combination thereof.

4. The nucleic acid-based assembly according to claim 1 or claim 2, wherein each of the at least one lipid is selected from the group comprising C₈₋₂₄ saturated or unsaturated fatty acids.

5. The nucleic acid-based assembly according to claim 1 or claim 2, wherein each of the at least one lipid comprises at least 2, 3, 4, 5, 6, 7, 8, 9, 10, 11, 12, 13, 14, 15, 16, 17, 18, 19, 20, 21, 22, 23, or 24 carbon atoms.

6. The nucleic acid-based assembly according to claim 1 or claim 2, wherein each of the at least one lipid is selected from the group consisting of C₈, C₁₀, C₁₂, C₁₄, C₁₆, C₁₈, C₂₀, C₂₂, and C₂₄ saturated and unsaturated fatty acid chains, and any combination thereof.

7. The nucleic acid-based assembly according to claim 1 or claim 2, wherein each of the at least one lipid comprises a C₁₂-lipid chain.

8. The nucleic acid-based assembly according to claim 1 or claim 2, wherein the at least one aptamer and/or the at least one nucleic acid motif each comprise a terminal lipid modification.

9. The method of claim 8, wherein the terminal lipid modification comprises 1, 2, 3, 4, 5, 6, 7, 8, 9, or 10 lipids or at least 1, 2, 3, 4, 5, 6, 7, 8, 9, or 10 lipids.

10. The method of claim 8, wherein the terminal lipid modification comprises 3, 4, or 5 lipids.

11. The method of any one of claims 8-10, wherein the terminal lipid modification is attached to the 5′-end.

12. The nucleic acid-based assembly according to any one of the foregoing claims, wherein the at least one aptamer targets a tissue antigen, a cancer-antigen, a tumor-antigen, a cellular antigen, a membrane protein, a cellular receptor, a cell surface molecule, a lymphocyte-directing target, a growth factor, or any combination thereof.

13. The nucleic acid-based assembly according to claim 12, wherein the at least one aptamer targets at least one of 4-1BB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, EphB3, FAP, Fibronectin, Folate Receptor, Ganglioside GM3, GD2, glucocorticoid-induced tumor necrosis factor receptor (GITR), gp100, gpA33, GPNMB, ICOS, IGFIR, Integrin av, Integrin αvβ, KIR, LAG-3, Lewis Y, Mesothelin, c-MET, MN Carbonic anhydrase IX, MUC1, MUC16, Nectin-4, NKGD2, NOTCH, OX40, OX40L, PD-1, PDL1, PSCA, PSMA, RANKL, RORI, ROR2, SLC44A4, Syndecan-1, TACI, TAG-72, Tenascin, TIM3, TRAILR1, TRAILR2, VEGFR-1, VEGFR-2, VEGFR-3, and any combination thereof.

14. The nucleic acid-based assembly according to claim 12 or claim 13, wherein the at least one aptamer comprises more than one aptamer, targets more than one antigen, or both.

15. The nucleic acid-based assembly according to any one of claims 12-14, wherein the at least one aptamer targets the hepatocyte growth factor receptor (cMET), wherein optionally the at least one aptamer comprises the sequence SEQ ID NO: 1 or a functional variant thereof.

16. The nucleic acid-based assembly according to any one of the foregoing claims, wherein the at least one nucleic acid motif comprises a motif that forms at least one hairpin loop.

17. The nucleic acid-based assembly according to claim 16, wherein the motif that forms the at least one hairpin loop comprises a 5′-GC rich oligodeoxynucleotide.

18. The nucleic acid-based assembly according to claim 16 or claim 17, wherein the at least one hairpin loop intercalates the drug.

19. The nucleic acid-based assembly according to any one of the foregoing claims, wherein the photo-responsive moiety comprises an azobenzene group, wherein optionally the azobenzene group comprises a 2′-methylazobenzene.

20. The nucleic acid-based assembly according to claim 19, wherein the 2′-methylazobenzene comprises 2′,6′-dimethylazobenzene.

21. The nucleic acid-based assembly according to any one of the foregoing claims, wherein the nucleic acid motif comprises the nucleotide sequence 5′-GCNGCGNCTCNGCGNCGATTATTACGCGCGAGCGCGC-3′ (SEQ ID NO: 2) or a functional variant thereof.

22. The nucleic acid-based assembly according to claim 21, wherein N is a 2′,6′-dimethylazobenzene-D-threoninol residue.

23. The nucleic acid-based assembly according to any one of the foregoing claims, wherein the drug comprises a regulatory molecule, an antagomir, a small interfering RNA, a microRNA, a pharmaceutical drug, or any combination thereof.

24. The nucleic acid-based assembly according to claim 23, wherein the drug comprises an anti-cancer drug or cocktail thereof.

25. The nucleic acid-based assembly according to claim 23 or claim 24, wherein the drug comprises a planar aromatic therapeutic agent.

26. The nucleic acid-based assembly according to claim 25, wherein the drug comprises doxorubicin.

27. The nucleic acid-based assembly according to any one of the foregoing claims, wherein the drug is released upon irradiation by visible light, ultraviolet light, or X-ray.

28. The nucleic acid-based assembly according to any one of the foregoing claims, wherein the at least one aptamer and the at least one nucleic acid motif are present in the assembly in a ratio in a range from ≥1:10 to ≤10:1, ≥1:5 to ≤5:1, or ≥1:2 to ≤3:2, wherein optionally the ratio is 1:1.

29. Use of the nucleic acid-based assembly according to any one of the foregoing claims as a medicament.

30. The use according the claim 29, wherein the medicament is for use in the treatment of cancer, wherein optionally the cancer comprises a solid tumor.

31. The use according to claim 30, wherein the cancer comprises an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumors (GIST), glioblastoma, head and neck squamous carcinoma, leukemia, liver hepatocellular carcinoma, low grade glioma, lung bronchioloalveolar carcinoma (BAC), lung non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, uveal melanoma, or any combination thereof.

32. Use of a nucleic acid-based assembly according to any one of claims 1 to 28 for the manufacture of a medicament.

33. The use according the claim 32, wherein the medicament is for the treatment of cancer, wherein optionally the cancer comprises a solid tumor.

34. The use according to claim 33, wherein the cancer comprises an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumors (GIST), glioblastoma, head and neck squamous carcinoma, leukemia, liver hepatocellular carcinoma, low grade glioma, lung bronchioloalveolar carcinoma (BAC), lung non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, uveal melanoma, or any combination thereof.

35. A pharmaceutical composition comprising as an active ingredient a nucleic acid-based assembly according to any one of claims 1 to 28.

36. The pharmaceutical composition according to claim 35 for use in the treatment of cancer.

37. A method of delivering a drug to a cell, comprising contacting the cell with a nucleic acid-based assembly according to any one of claims 1 to 28 and irradiating the cell.

38. The method according to claim 37, wherein delivery of the drug to the cell kills the cell.

39. The method according to claim 37 or claim 38, wherein the cell comprises a cultured cell, a diseased cell, a tumor cell, a cancer cell, or any combination thereof.

40. The method of claim 39, wherein the cancer comprises an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumors (GIST), glioblastoma, head and neck squamous carcinoma (BAC), lung non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, uveal melanoma, or any combination thereof.

41. A method of treating a disease or disorder in a subject in need thereof, the method comprising administering to the subject a therapeutically effective amount of the pharmaceutical composition of any one of claims 35 to 36.

42. The method of claim 41, wherein the disease or disorder comprises a cancer.

43. The method of claim 42, wherein the cancer comprises an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumors (GIST), glioblastoma, head and neck squamous carcinoma, leukemia, liver hepatocellular carcinoma, low grade glioma, lung bronchioloalveolar carcinoma (BAC), lung non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, uveal melanoma, or any combination thereof.
